(19) United States
(12) Patent Application Publication
(10) Pub. No.: US 2025/0289837 A1
(43) Pub. Date: Sep. 18, 2025

(54) MITOCHONDRIOTROPIC HETEROARYL BENZAMIDE POTASSIUM CHANNEL KV1.3 INHIBITORS

(71) Applicants: UNIVERZA V LJUBLJANI, Ljubljana (SI); Max-Planck-Gesellschaft zur Förderung der Wissenschaften e.V., München (DE)

(72) Inventors: Lucija Peterlin Mašic, Ljublijana (SI); Tihomir Tomašic, Ljublijana (SI); Spela Gubic, Ljublijana (SI); Luis A. Pardo, München (DE)

(21) Appl. No.: 18/858,781

(22) PCT Filed: Apr. 21, 2023
(86) PCT No.: PCT/EP2023/060402

§ 371 Date: Oct. 22, 2024

(30) Foreign Application Priority Data
Apr. 22, 2022 (LU) LU501901

(51) Int. Cl. C07F 9/6553; A61K 31/67; A61P 35/00; C07F 9/54

(52) U.S. Cl. CPC C07F 9/655354; A61K 31/67; A61P 35/00; C07F 9/5442; C07B 2200/07

(57) ABSTRACT

The present invention relates to compounds of formula (I), processes for their preparation, and pharmaceutical compositions containing them as the active ingredient. Compounds of the present invention may be useful as mitochondrial KV1.3 inhibitors (mitoKV1.3) to treat cancer diseases and the like, including breast, colon, and prostate tumors, melanoma, smooth muscle, and skeletal muscle cancer, chronic lymphocytic leukemia, glioblastoma, and pancreatic ductal adenocarcinoma.

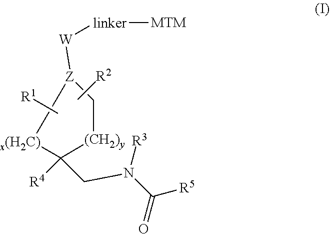

### FIELD OF THE INVENTION

The present invention relates to compounds of formula (I), processes for their preparation, and pharmaceutical compositions containing them as the active ingredient. Compounds of the present invention may be useful as mitochondrial K_{V}1.3 inhibitors (mitoK_{V}1.3) to treat cancer diseases and the like, including breast, colon, and prostate tumors, melanoma, smooth muscle, and skeletal muscle cancer, chronic lymphocytic leukemia, glioblastoma, and pancreatic ductal adenocarcinoma.

### BACKGROUND ART

Potassium channels are transmembrane proteins that facilitate the passage of potassium ions through the plasma membrane along their electrochemical gradient. They have been classified according to their biophysical and pharmacological characteristics (for nomenclature, see Gutman et al., Pharmacol Rev, 55(4), 583-6, 2003). Salient among these are the voltage-gated potassium channels, globally termed K_{V}. The largest voltage-gated potassium channel subfamily, K_{V}1 (Shaker), comprises eight voltage-gated potassium channels K_{V}1.1-K_{V}1.8 (Yellen et al., Nature, 419, 35-42, 2002).

K_{V}1.3 is localized at the cell plasma membrane, the inner mitochondrial membrane (mitoK_{V}1.3), the nuclear membrane, and the membrane of the cis-Golgi apparatus (Perez-Garcia et al., Am J Physiol 314, C27-C40, 2018). K_{V}1.3 expression has been detected in the mitochondria of lymphocytes, where the channel participates in the regulation of the mitochondrial membrane potential (Δψ) (Szabò et al., J Biol Chem, 280, 12790-8, 2005). Flow of potassium ions along the electrochemical gradient into the mitochondrial matrix promotes membrane depolarization and, oppositely, block of these channels causes hyperpolarization (Szabo et al., Proc Natl Acad Sci USA, 105, 14861-6, 2008). Intact mitoK_{V}1.3 function is also required for matrix volume regulation and ROS production (Checchetto et al., Biochem Biophys Res Commun, 500, 51-8, 2018). MitoK_{V}1.3 channels show pharmacological and biophysical properties indistinguishable from those in the plasma membrane, therefore both isoforms of the channel are likely encoded by the same gene (Prosdocimi et al., SLAS Discov, 24, 882-892, 2019). Blockade of mitoK_{V}1.3 by Bax plays a relevant role in promoting apoptosis in several types of tumor cells; therefore, it is considered a potential pharmacological target for cancer treatment (Szabo et al., Redox Biology, 42, 101846, 2021).

### Targeting Mitochondrial K_{V}1.3 Channels for Cancer Therapy

Cancer remains one of the leading causes of mortality worldwide, therefore there is an urgent need to develop novel specific anticancer treatments, which could be combined with traditional chemotherapy to prolong survival and increase patient quality of life. Understanding the molecular mechanisms of cancer progression and exploiting the differences between cancer and normal cells are essential in terms of discovering novel molecular targets in cancer therapy. Cancer cells undergo multiple mutations that endorse them with unlimited proliferation and invulnerability to apoptosis (Serrano-Novillo, C. et al., Cancers, 11, 287, 2019). Novel cancer drugs need to specifically target these tumor features, which are otherwise absent in normal cells (Arcangeli et al., Curr Med Chem, 16, 66-93, 2009).

Pharmacological targeting of mitochondrial ion channels is emerging as a promising approach to eliminate cancer cells; as most of these channels are differentially expressed and/or regulated in cancer cells in comparison to healthy ones, this strategy may selectively eliminate the former (Szabo et al., Redox Biology, 42, 101846, 2021). Perturbation of ion fluxes across the outer and inner membranes is linked to alterations of redox state, membrane potential and bioenergetic efficiency. This leads to indirect modulation of oxidative phosphorylation, which is/may be fundamental for both cancer and cancer stem cell survival. Furthermore, given the crucial contribution of mitochondria to the intrinsic apoptosis pathway, modulation of their ion channels leading to cytochrome c release may be of great advantage in case of resistance to drugs triggering apoptotic events upstream of the mitochondrial phase. Mitochondria play a central role in cancer development, by contributing to most of the classical hallmarks of cancer, including sustained proliferation, metabolic re-programming, apoptosis resistance, invasion and induction of angiogenesis. In addition, mitochondria affect also the function of anti- and pro-tumoral immune cells in the tumor microenvironment (Hanahan et al., Cell, 144, 646-674, 2011). Limitless cell proliferation, typical of cancer cells is supported by mitochondrial function, since tumor cells need to upregulate macromolecular biosynthesis while maintaining energy production (Trotta et al., Cell Mol Life Sci, 74, 1999-2017, 2017).

The voltage-gated Shaker-type potassium channels Kv1.3 are overexpressed in various primary cancer cells (e.g., B-CLL) and tissues as well as in cancer cell lines (Bielanska et al., Curr. Cancer Drug Targets, 9, 904-914, 2009). The channel has been realized as an arising tumor marker, but a clear pattern for altered K_{V}1.3 expression in cancer cells versus healthy cells has not been established yet, as type, and stage of disease also influence the K_{V}1.3 expression (Comes et al., Front Physiol, 4, 283, 2013). Nevertheless, channels are aberrantly expressed in breast, colon, and prostate tumors, smooth muscle, and skeletal muscle cancer, and in mature neoplastic B cells in chronic lymphocytic leukemia, and this apparently correlates with their expression in mitochondria (Comes et al., Biochim Biophys Acta BBA—Biomembr, 1848, 2477-92, 2015). MitoK_{V}1.3 has recently emerged as novel molecular target for anticancer therapy, as it might be involved in promoting cancer cell proliferation. Inhibition of mitoK_{V}1.3 at sub-lethal concentrations affected cell cycle progression and cell proliferation. The drugs inhibiting this channel slightly increased the percentage of cells in S phase and decreased the population of cells at the G0/G1 phases of the cell cycle, presumably related to slightly increased ROS levels within the cells (Peruzzo et al., Front Oncol, 7, 239, 2017). On the other hand, sub-lethal concentrations of the same Kv1.3 inhibitors reduced Wnt signaling, which plays an important role in the uncontrolled proliferation of cancer cells when it is constitutively activated (Costa et al., Cell Rep, 28, 1949-1960, 2019). Moreover, the channel is significantly involved in apoptotic signaling of cancer and normal cells (Bachmann et al., Cell Physiol Biochem, 53(S1), 63-78, 2019). Induction of apoptosis in cancer cells by mitoK_{V}1.3 inhibition might be considered as an effective method to selectively kill cancer cells (Teisseyre et al., Adv Clin Exp Med, 24, 517-524, 2015). When proteins of the Bcl-2 family, such as Bcl-2-like protein 4 (Bax), inhibit K_{V}1.3 located in the inner mitochondrial membrane, they activate the intrinsic apoptotic pathway, which is characterized by transient membrane hyperpolarization, elevated ROS production, cytochrome c release, and finally membrane depolarization. Moreover, membrane permeable K_{V}1.3 inhibitors can mimic this Bax interaction via specific inhibitory action on K_{V}1.3 and their selectivity for cancer over healthy cells depends on the synergy of the high K_{V}1.3 expression and altered basal redox state in cancer cells (Checchetto et al., Cell Physiol Biochem, 53, 52-62, 2019). Psoralen analogue PAP-1 (4-(4-phenoxybutoxy)-7H-furo[3,2-g]chromen-7-one) is potent (IC₅₀=2 nM) and selective (i.e. 23-fold over Kv1.5) small-molecule K_{V}1.3 inhibitor (Schmitz et al., Mol Pharmacol, 68, 1254-1270, 2005). Psora-4 (4-(4-Phenylbutoxy)-7H-furo[3,2-g][1]benzopyran-7-one) is another potent psoralen-based K_{V}1.3 inhibitor (IC₅₀=3 nM), which lacked selectivity over K_{V}1.5 (IC₅₀=7.7 nM) (Vennekamp et al., Mol Pharmacol, 65, 1364-1374, 2004). The antimycobacterial drug clofazimine (N,5-bis(4-chlorophenyl)-3-propan-2-yliminophenazin-2-amine) is a well-studied K_{V}1.3 inhibitor (IC₅₀=300 nM) with tenfold selectivity over K_{V}1.1, K_{V}1.2, K_{V}1.5 and K_{V}3.1 (Ren et al., PLOS One, 3, e4009, 2008). PAP-1, Psora-4, and clofazimine induced apoptosis in cancer cells (human SAOS2, mouse B16F10 melanoma, and human B-CLL) via their specific inhibitory action on the mitoK_{V}1.3 (Leanza et al., EMBO Mol Med, 4, 577-593, 2012), while they did not show similar effects on healthy cells (HEK293 and K562 cells, and T and B cells from healthy subjects). Clofazimine induced apoptosis in pancreatic ductal adenocarcinoma (PDAC) cells; moreover, it significantly reduced the primary tumor weight in an orthotopic PDAC xenograft model in the SCID beige mouse model (Zaccagnino et al., Oncotarget, 8, 38276-93, 2016) and reduced the tumor size by 90% in a mouse model of the orthotopic melanoma B16F10 line (Leanza et al., EMBO Mol Med, 4, 577-93, 2012). Selective action of these channel inhibitors induced cell death of B-cells (B-lymphocytes) from patients suffering from chronic lymphocytic leukemia, which had elevated level of functional mitoKv1.3 channels compared to cells from healthy donors (Leanza, et al., Leukemia 27, 1782-5, 2013).

Mitochondriotropic K_{V}1.3 Channel Inhibitors that Specifically Target Inner Mitochondrial Membrane

The mitochondria of cancerous cells have emerged as oncological target, as they are involved in bioenergetic processes, such as the Krebs cycle and reactive oxygen species (ROS) production (Nguyen et al., Cancers, 29, 11(7), 916, 2019). Advantages of selective ligand-targeting to mitochondria are reducing off-target effects and drug dosage, thus use of mitoK_{V}1.3 inhibitors is expecting to maximize damage to cancer while minimizing damage to healthy cells (Szabo et al., Redox Biol, 42, 101846, 2021). Since mitochondria maintain high transmembrane potential, positively charged membrane-permeant substances tend to accumulate into the organelles to reach the electrochemical equilibrium. Two general strategies have been described that took advantage of these unique mitochondria characteristics to specifically deliver drug to mitochondria compartments (Parrasia et al., Cell Physiol Biochem, 53, 11-43, 2019). The first one is based on structural modifications with the attachment of a mitochondria-targeting ligand to the pharmacologically active compound, and the second one uses nanocarriers, which can selectively transfer active compounds to mitochondrial compartments. The first strategy has been applied for the design of mitochondriotropic K_{V}1.3 inhibitors (Leanza et al., Cancer Cell, 31, 516-531, 2017). The specific mitochondria-targeting K_{V}1.3 inhibitors are lipophilic and membrane-permeable conjugates of the active K_{V}1.3 channel inhibitors and mitochondria-targeting cations such as TPP⁺. Cations are attached to the active compounds through a stable or labile linker (Battogtokh et al., Frontiers in Pharmacology, 9, 922, 2018). When mitochondriotropics with stable linker are dissolved in solution, the K_{V}1.3 channel inhibitor remains attached to the cation. However, with incorporation of a “cleavable linker” amide or ester bond as a linker, the initial prodrug is hydrolyzed to the pharmacologically active compound.

Mitochondriotropic K_{V}1.3 inhibitors were designed by linking a TPP⁺ moiety to PAP-1 (4-(4-phenoxybutoxy)-7H-furo[3,2-g]chromen-7-one) or its analogue PAPOH (4-(4-(4-hydroxyphenoxy)butoxy)-7H-furo[3,2-g]chromen-7-one) by a stable alkoxy ether linkage (PAPTP, (3-(4-(4-((7-oxo-7H-furo[3,2-g]chromen-4-yl)oxy)butoxy)phenyl)propyl)triphenylphosphonium iodide), or by labile linker comprising whether carbamate (PCARBTP, (3-(((4-(4-((7-oxo-7H-furo[3,2-g]chromen-4-yl)oxy)butoxy)phenoxy)carbonyl)amino)propyl)triphenylphosphonium iodide) or carbonate moiety (PCTP, (3-(((4-(4-((7-oxo-7H-furo[3,2-g]chromen-4-yl)oxy)butoxy)phenoxy)carbonyl)oxy)propyl)triphenylphosphonium iodide). PCARBTP was hydrolyzed to PAPOH under physiological conditions in mouse blood and went through slow hydrolysis in Dulbecco's modified Eagle medium (culture medium for mammalian cells) to produce PAPOH (Leanza et al., Cancer Cell, 31, 516-531, 2017, Mattarei et al., Front Oncol, 8, 122, 2018). Mitochondriotropic psoralens PAPTP, PCARBTP, and PCTP were much more effective than the parent PAP-1 in in vitro and in vivo studies and selectively acted on cancer cells, while sparing normal cells and tissues (Szabo et al., Redox Biol, 42, 101846, 2021). They induced cell death in K_{V}1.3-expressing Jurkat leukemia T cells, in mouse melanoma B16F10 cells and in four human pancreatic ductal adenocarcinoma cell types (Bx-PC3, PANC-1, As-PC1, CAPAN-1 cells). Compounds PAPTP and PCARBTP greatly reduce the B16F10 tumor volume in vivo using an orthotopic mouse B16F10 melanoma model. Moreover, PAPTP co-applied with cisplatin achieved more than 90% tumor volume reduction, which indicated the possibility of synergy with other chemotherapeutic drugs (Leanza et al., Cancer Cell, 31, 516-531, 2017, Mattarei et al., Front Oncol, 8, 122, 2018). Treating mice with PCARBTP and PAPTP resulted in significant reductions in human pancreatic Colo357 tumors.

### SUMMARY OF THE INVENTION

The present invention is based on a new class of compounds that are useful for inhibiting mitochondrial K_{V}1.3 ion channels. The compounds of the present invention are effective for the treatment of cancer including metastatic cancer and chemoresistance.

More specifically, the present invention relates in a main aspect to a compound of formula (I):

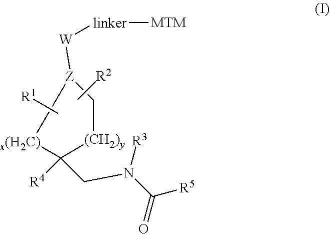

wherein R¹, R², R³, R⁴, R⁵, x, y, Z, W, linker and MTM (mitochondria targeting moiety) are as defined herein, and their pharmaceutically acceptable salts, racemates, diastereomers, enantiomers, esters, carbamates, sulphates, phosphates and prodrugs thereof.

In other aspects, the present invention provides pharmaceutical compositions comprising a compound of formula (I) or any of its various embodiments.

Also encompassed by the present invention is the use of compounds of formula (I) or any of its various embodiments in the preparation of a medicament, as well as methods for treatment employing compounds of formula (I) or any of its various embodiments.

The various aspects of the present invention, including the compound of formula (I) and its various embodiments, are described in more detail below. It should become clear that any details described in connection with one aspect of the present invention applies mutatis mutandis to any other aspect of the present invention.

The present invention can be summarized by the following items:
1. A compound of formula (I):

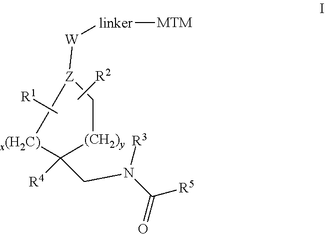

wherein:
“MTM” is a mitochondria targeting moiety;
x and y are independently 0, 1, or 2;
Z is CH or N; preferably Z is CH;
R¹ and R² are independently selected from the group consisting of hydrogen, halo, wherein halo is fluoro, chloro, bromo, or iodo, hydroxy, HO(C₁-C₆)-alkyloxy, (C₁-C₄)-perfluoroalkyl, O(CO)CCl₃, (C₁-C₅)-alkyl-S(O)ₙ—, phenyl-(CH₂)ᵣ—S(O)ₙ—, cyano, nitro, COOH, CO(C₁-C₆)-alkyl, COO(C₁-C₆)-alkyl, CONR⁶R⁷, NR⁶R⁷, O(CO)NR⁶R⁷, azido, NR⁶(CO)NR⁶R⁷, (C₁-C₁₀)-alkyl, (C₂-C₁₀)-alkenyl, (C₂-C₁₀)-alkynyl, O[(C═O)Oᵣ]ₛ(C₁-C₆)-alkyl, O[(C═O)Oᵣ]ₛ(C₂-C₆)-alkenyl, O[(C═O)Oᵣ]ₛaryl, O[(C═O)Oᵣ]ₛheteroaryl, O(CH₂)ₙheteroaryl, aryl, O(CH₂)ₙaryl, oxo, ═CH—(C₁-C₆)-alkyl, ═CH—(C₂-C₆)-alkenyl, ═CH-aryl, and ═CH₂, with r and s at each occurrence being independently from each other 0 or 1, and n at each occurrence being 0, 1, 2 or 3, preferably n at each occurrence being 0 or 1;
R³ is hydrogen, [(C═O)Oᵣ]ₛaryl or [(C═O)Oᵣ]ₛ(C₁-C₆)-alkyl, with r and s at each occurrence being independently from each other 0 or 1;
R⁴ and R⁵ are independently selected from the group consisting of substituted or unsubstituted aryl, such as phenyl or naphtyl, and substituted or unsubstituted five or six membered heterocyclyl containing from 1 to 3 heteroatoms selected from the group consisting of O, N and S, such as a five or six membered aromatic heterocyclyl containing from 1 to 3 heteroatoms selected from the group consisting of O, N and S;
wherein the aryl, if substituted, is substituted with one or more (such as one or two) substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3, preferably n at each occurrence being 0 or 1;
wherein the heterocyclyl, if substituted, is substituted with one or more (such as one or two) substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3, preferably n at each occurrence being 0 or 1;
R⁶ and R⁷ are independently selected from the group consisting of hydrogen, [(C═O)Oᵣ]ₛaryl, [(C═O)Oᵣ]ₛ(C₂-C₈)-alkenyl, [(C═O)Oᵣ]ₛ(C₁-C₈)-alkyl, (C═O)ᵣS(O)ₙ(C₁-C₈)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1;
W is a suitable functional group depending on the available site on the particular K_{V}1.3 inhibitor of interest which is attached to the linker;
Linker is selected from:
—(C(R⁹)(R¹⁰))_{I}—, wherein I is from 1 to 20, preferably from 1 to 10, more preferably 3 to 5; and R⁹ and R¹⁰ are independently of one another —H, halogen, —CF₃, —OH, —(C₁-C₆)-alkyl, —OC(O)(C₁-C₆)-alkyl, [(C═O)Oᵣ]ₛaryl, [(C═O)Oᵣ]ₛheteroaryl, or [(C═O)Oᵣ]ₛ(C₁-C₆)-alkyl, with r and s at each occurrence being independently from each other 0 or 1;
Non-peptidic polymeric linkers, such as non-peptidic polymeric linkers selected from polyalkylene oxides (e.g. polyethylene glycol, polypropylene glycol, and the like), polyvinyl alcohol, polyvinylpyrrolidone as well as derivatives and copolymers thereof;
Non-polymeric aliphatic linkers, such as non-polymeric aliphatic linkers comprising a divalent, linear or branched, straight or cyclic, saturated or unsaturated hydrocarbon chain having from 2 to 20 carbon atoms, wherein the carbon atoms are optionally replaced by a group selected from —O—, —S—, —NH—, —C(═O)—, —OC(═O)—, —N(C₁-C₆ alkyl)-, NHC(═O)—, —N(C₁-C₆ alkyl)C(═O)—, —S(═O)— or —S(═O)₂— and wherein the chain is optionally substituted on carbon with one or more (e.g. 1, 2, 3 or 4) substituents;
A divalent radical formed from an amino acid or peptide; and

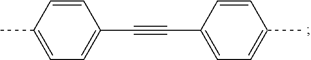

or a pharmaceutically acceptable salt, racemate, diastereomer, enantiomer, ester, carbamate, sulphate, phosphate or prodrug thereof.
2. Compound according to item 1, wherein MTM is a mitochondria targeting moiety selected from:

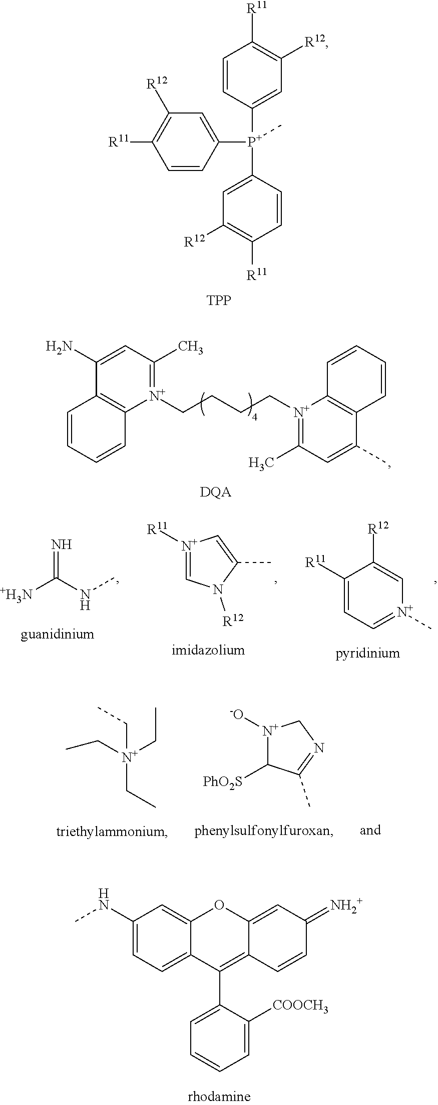

wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.
3. Compound according to item 1, wherein MTM is

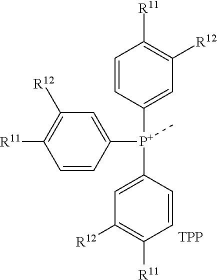

wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.
4. Compound according to item 1, having structural Formula II or Formula III

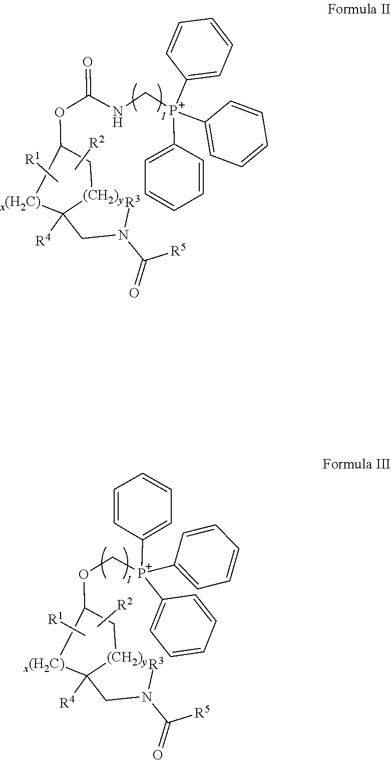

wherein I is from 1 to 20, preferably from 1 to 10, more preferably 3 to 5.
5. Compound according to item 1, having structural Formula IV or Formula V

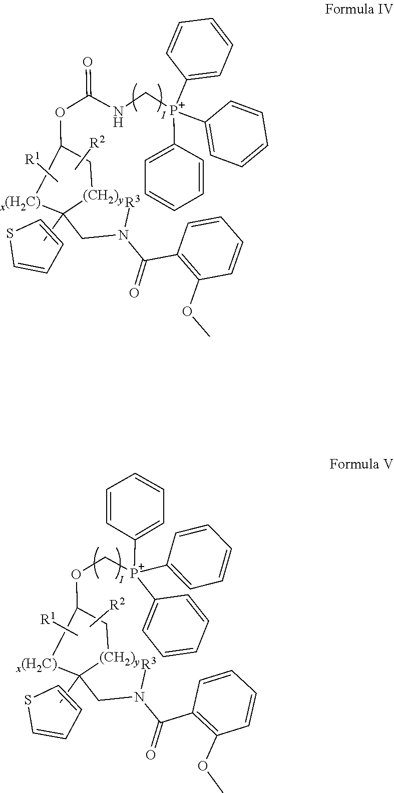

wherein I is from 1 to 20, preferably from 1 to 10, more preferably 3 to 5.
6. Compound according to item 1, having structural Formula VI or Formula VII

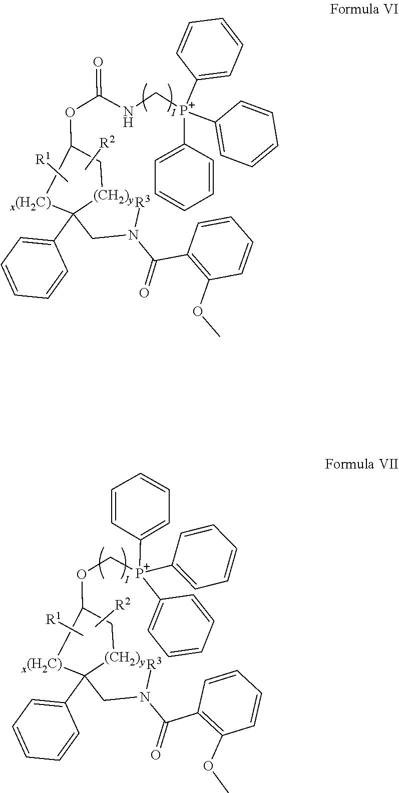

wherein I is from 1 to 20, preferably from 1 to 10, more preferably 3 to 5.
7. Compound according to item 1, having structural Formula VIII or Formula IX

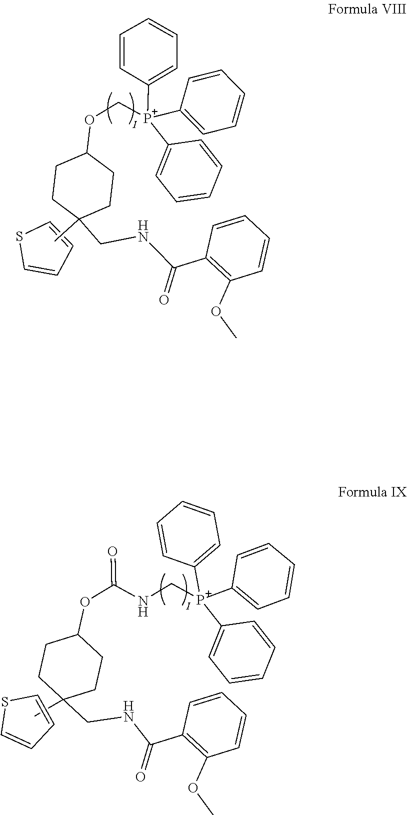

wherein I is from 1 to 20, preferably from 1 to 10, more preferably 3 to 5.
8. Compound according to any one of items 1 to 3, being an enantiomerically pure compound or an enantiomerically enriched compound with the following structural Formula X or Formula XI

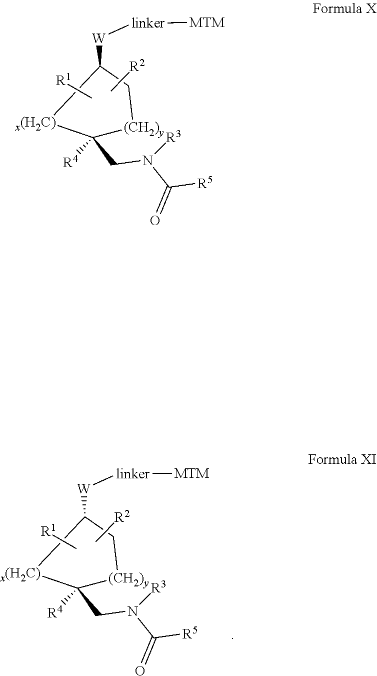

9. Compound according to item 1, 2, 3 or 8, wherein W comprises a cleavable group, such as a cleavable group selected from esters, carbamates, disulfide linkers, oxime linkers, hydrazine groups, diazolinkers, carbonyloxyethylsulfone groups, amino acid groups, and phenylacetamide groups.
10. Compound according to item 1, 2, 3 or 8, wherein W is selected from (C₁-C₆)-alkyl, (C₂-C₆)-alkenyl, (C₂-C₆)-alkynyl, (C₃-C₆)cycloalkyl, aryl, heteroaryl, —OC(O)NR⁸—, —COO—, —OC(O)—, —CONR⁸—, —NHR⁸—, —SO—, —SO₂NR⁸—, —CHR⁸—, —SO₂—, —CO—, —S—, —O—, —CH₂—, —OC(O)—CH₂—C(O)O—, and —CH(OH)—CH(OH)—;
   wherein R⁸ is —H, —F, —Cl, —Br, —OH, —(C₁-C₆)-alkyl, or —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.
11. The compound of any one of items 1 to 6 and 8 to 10, wherein x is 0.
12. The compound of any one of items 1 to 6 and 8 to 10, wherein x is 1.
13. The compound of any one of items 1 to 6 and 8 to 10, wherein x is 2.
14. The compound of any one of items 1 to 6, and 8 to 13, wherein y is 0.
15. The compound of any one of items 1 to 6, and 8 to 13, wherein y is 1.
16. The compound of any one of items 1 to 6, and 8 to 13, wherein y is 2.
17. The compound of any one of items 1 to 6, and 8 to 13, wherein x is 2 and y is 1.
18. The compound of any one of items 1 to 17, wherein R¹ is hydrogen.
19. The compound of any one of items 1 to 17, wherein R¹ is hydroxyl.
20. The compound of any one of items 1 to 17, wherein R¹ is HO(C₁-C₆)-alkyloxy.
21. The compound of any one of items 1 to 17, wherein R¹ is (C₁-C₄)-perfluoroalkyl.
22. The compound of any one of items 1 to 17, wherein R¹ is O(CO)CCl₃.
23. The compound of any one of items 1 to 17, wherein R¹ is (C₁-C₆)-alkyl-S(O)ₙ—.
24. The compound of any one of items 1 to 17, wherein R¹ is phenyl-(CH₂)ᵣ—S(O)ₙ—.
25. The compound of any one of items 1 to 17, wherein R¹ is cyano.
26. The compound of any one of items 1 to 17, wherein R¹ is nitro.
27. The compound of any one of items 1 to 17, wherein R¹ is COOH.
28. The compound of any one of items 1 to 17, wherein R¹ is CO(C₁-C₆)-alkyl.
29. The compound of any one of items 1 to 17, wherein R¹ is COO(C₁-C₆)-alkyl.
30. The compound of any one of items 1 to 17, wherein R¹ is CONR⁶R⁷.
31. The compound of any one of items 1 to 17, wherein R¹ is NR⁶R⁷.
32. The compound of any one of items 1 to 17, wherein R¹ is O(CO)NR⁶R⁷.
33. The compound of any one of items 1 to 17, wherein R¹ is azido.
34. The compound of any one of items 1 to 17, wherein R¹ is NR⁶(CO)NR⁶R⁷.
35. The compound of any one of items 1 to 17, wherein R¹ is (C₁-C₁₀)-alkyl.
36. The compound of any one of items 1 to 17, wherein R¹ is (C₂-C₁₀)-alkenyl.
37. The compound of any one of items 1 to 17, wherein R¹ is (C₂-C₁₀)-alkynyl.
38. The compound of any one of items 1 to 17, wherein R¹ is O[(C═O)Oᵣ]ₛ(C₁-C₆)-alkyl.
39. The compound of any one of items 1 to 17, wherein R¹ is O[(C═O)Oᵣ]ₛ(C₂-C₆)-alkenyl.
40. The compound of any one of items 1 to 17, wherein R¹ is O[(C═O)Oᵣ]ₛaryl.
41. The compound of any one of items 1 to 17, wherein R¹ is O[(C═O)Oᵣ]ₛheteroaryl.
42. The compound of any one of items 1 to 17, wherein R¹ is O(CH₂)ₙheteroaryl.
43. The compound of any one of items 1 to 17, wherein R¹ is aryl.
44. The compound of any one of items 1 to 17, wherein R¹ is O(CH₂)ₙaryl.
45. The compound of any one of items 1 to 17, wherein R¹ is oxo.
46. The compound of any one of items 1 to 17, wherein R¹ is ═CH—(C₁-C₆)-alkyl.
47. The compound of any one of items 1 to 17, wherein R¹ is ═CH—(C₂-C₆)-alkenyl-.
48. The compound of any one of items 1 to 17, wherein R¹ is ═CH-aryl.
49. The compound of any one of items 1 to 17, wherein R¹ is ═CH₂.
50. The compound of any one of items 1 to 17, wherein R¹ is halogen.
51. The compound of any one of items 1 to 50, wherein R² is hydrogen.
52. The compound of any one of items 1 to 50, wherein R² is hydroxyl.
53. The compound of any one of items 1 to 50, wherein R² is halogen.
54. The compound of any one of items 1 to 50, wherein R² is HO(C₁-C₆)-alkyloxy.
55. The compound of any one of items 1 to 50, wherein R² is (C₁-C₄)-perfluoroalkyl.
56. The compound of any one of items 1 to 50, wherein R² is O(CO)CCl₃.
57. The compound of any one of items 1 to 50, wherein R² is (C₁-C₆)-alkyl-S(O)ₙ—.
58. The compound of any one of items 1 to 50, wherein R² is phenyl-(CH₂)ᵣ—S(O)ₙ—.
59. The compound of any one of items 1 to 50, wherein R² is cyano.
60. The compound of any one of items 1 to 50, wherein R² is nitro.
61. The compound of any one of items 1 to 50, wherein R² is COOH.
62. The compound of any one of items 1 to 50, wherein R² is CO(C₁-C₆)-alkyl.
63. The compound of any one of items 1 to 50, wherein R² is COO(C₁-C₆)-alkyl.
64. The compound of any one of items 1 to 50, wherein R² is CONR⁶R⁷.
65. The compound of any one of items 1 to 51, wherein R² is NR⁶R⁷.
66. The compound of any one of items 1 to 51, wherein R² is O(CO)NR⁶R⁷.
67. The compound of any one of items 1 to 51, wherein R² is azido.
68. The compound of any one of items 1 to 51, wherein R² is NR⁶(CO)NR⁶R⁷.
69. The compound of any one of items 1 to 51, wherein R² is (C₁-C₁₀)-alkyl.
70. The compound of any one of items 1 to 51, wherein R² is (C₂-C₁₀)-alkenyl.
71. The compound of any one of items 1 to 51, wherein R² is (C₂-C₁₀)-alkynyl.
72. The compound of any one of items 1 to 51, wherein R² is O[(C═O)Oᵣ]ₛ(C₁-C₆)-alkyl.
73. The compound of any one of items 1 to 51, wherein R² is O[(C═O)Oᵣ]ₛ(C₂-C₆)-alkenyl.
74. The compound of any one of items 1 to 51, wherein R² is O[(C═O)Oᵣ]ₛaryl.
75. The compound of any one of items 1 to 51, wherein R² is O[(C═O)Oᵣ]ₛheteroaryl.
76. The compound of any one of items 1 to 51, wherein R² is O(CH₂)ₙheteroaryl.
77. The compound of any one of items 1 to 51, wherein R² is aryl.
78. The compound of any one of items 1 to 51, wherein R² is O(CH₂)ₙaryl.
79. The compound of any one of items 1 to 51, wherein R² is oxo.
80. The compound of any one of items 1 to 51, wherein R² is ═CH—(C₁-C₆)-alkyl.
81. The compound of any one of items 1 to 51, wherein R² is ═CH—(C₂-C₆)-alkenyl-.
82. The compound of any one of items 1 to 51, wherein R² is ═CH-aryl.
83. The compound of any one of items 1 to 51, wherein R² is ═CH₂.
84. The compound of any one of items 1 to 17, wherein R¹ and R² are each hydrogen.
85. The compound of any one of items 1 to 84, wherein R³ is hydrogen.
86. The compound of any one of items 1 to 84, wherein R³ is [(C═O)Oᵣ]ₛaryl.
87. The compound of any one of items 1 to 84, wherein R³ is [(C═O)Oᵣ]ₛ(C₁-C₆)-alkyl.
88. The compound of any one of items 1 to 87, wherein R⁴ is a substituted or unsubstituted aryl.
89. The compound of item 88, wherein the aryl is phenyl.
90. The compound of item 88, wherein the aryl is naphthyl.
91. The compound of any one of items 89 to 90, wherein the aryl is unsubstituted.
91. The compound of any one of items 89 to 90, wherein the aryl is monosubstituted.
92. The compound of any one of items 89 to 90, wherein the aryl is disubstituted.
93. The compound of any one of items 1 to 87, wherein R⁴ is a substituted or unsubstituted five or six membered heterocyclyl containing from 1 to 3 heteroatoms selected from the group consisting of O, N and S.
94. The compound of any one of items 1 to 87, wherein R⁴ is a substituted or unsubstituted five membered heterocyclyl containing from 1 to 3 heteroatoms selected from the group consisting of O, N and S.
95. The compound of any one of items 1 to 87, wherein R⁴ is a substituted or unsubstituted six membered heterocyclyl containing from 1 to 3 heteroatoms selected from the group consisting of O, N and S.
96. The compound of any one of items 1 to 87, wherein R⁴ is an unsubstituted five or six membered heterocyclyl containing from 1 to 3 heteroatoms selected from the group consisting of O, N and S.
97. The compound of any one of items 1 to 87, wherein R⁴ is an unsubstituted five membered heterocyclyl containing from 1 to 3 heteroatoms selected from the group consisting of O, N and S.
98. The compound of any one of items 1 to 87, wherein R⁴ is an unsubstituted six membered heterocyclyl containing from 1 to 3 heteroatoms selected from the group consisting of O, N and S.
99. The compound of any one of items 1 to 87, wherein R⁴ is a substituted five or six membered heterocyclyl containing from 1 to 3 heteroatoms selected from the group consisting of O, N and S.
100. The compound of any one of items 1 to 87, wherein R⁴ is a monosubstituted five or six membered heterocyclyl containing from 1 to 3 heteroatoms selected from the group consisting of O, N and S.
101. The compound of any one of items 1 to 87, wherein R⁴ is a monosubstituted five membered heterocyclyl containing from 1 to 3 heteroatoms selected from the group consisting of O, N and S.
102. The compound of any one of items 1 to 87, wherein R⁴ is a monosubstituted six membered heterocyclyl containing from 1 to 3 heteroatoms selected from the group consisting of O, N and S.
103. The compound of any one of items 1 to 87, wherein R⁴ is a disubstituted five or six membered heterocyclyl containing from 1 to 3 heteroatoms selected from the group consisting of O, N and S.
104. The compound of any one of items 1 to 87, wherein R⁴ is a disubstituted five membered heterocyclyl containing from 1 to 3 heteroatoms selected from the group consisting of O, N and S.
105. The compound of any one of items 1 to 87, wherein R⁴ is a disubstituted six membered heterocyclyl containing from 1 to 3 heteroatoms selected from the group consisting of O, N and S.
106. The compound of any one of items 1 to 87, wherein R⁴ is an unsubstituted, monosubstituted, or disubstituted thiophene.
107. The compound of any one of items 1 to 87, wherein R⁴ is a 2- or 3-substituted thiophene.
108. The compound of any one of items 1 to 107, wherein R⁵ is a substituted or unsubstituted aryl.
109. The compound of item 108, wherein the aryl is phenyl.
110. The compound of item 108, wherein the aryl is naphthyl.
111. The compound of any one of items 108 to 110, wherein the aryl is unsubstituted.
112. The compound of any one of items 108 to 110, wherein the aryl is monosubstituted.
113. The compound of any one of items 108 to 110, wherein the aryl is disubstituted.
114. The compound of any one of items 1 to 107, wherein R⁵ is a substituted or unsubstituted five or six membered heterocyclyl containing from 1 to 3 heteroatoms selected from the group consisting of O, N and S.
115. The compound of any one of items 1 to 107, wherein R⁵ is a substituted or unsubstituted five membered heterocyclyl containing from 1 to 3 heteroatoms selected from the group consisting of O, N and S.
116. The compound of any one of items 1 to 107, wherein R⁵ is a substituted or unsubstituted six membered heterocyclyl containing from 1 to 3 heteroatoms selected from the group consisting of O, N and S.
117. The compound of any one of items 1 to 107, wherein R⁵ is an unsubstituted five or six membered heterocyclyl containing from 1 to 3 heteroatoms selected from the group consisting of O, N and S.
118. The compound of any one of items 1 to 107, wherein R⁵ is an unsubstituted five membered heterocyclyl containing from 1 to 3 heteroatoms selected from the group consisting of O, N and S.
119. The compound of any one of items 1 to 107, wherein R⁵ is an unsubstituted six membered heterocyclyl containing from 1 to 3 heteroatoms selected from the group consisting of O, N and S.
120. The compound of any one of items 1 to 107, wherein R⁵ is a substituted five or six membered heterocyclyl containing from 1 to 3 heteroatoms selected from the group consisting of O, N and S.
121. The compound of any one of items 1 to 107, wherein R⁵ is a monosubstituted five or six membered heterocyclyl containing from 1 to 3 heteroatoms selected from the group consisting of O, N and S.
122. The compound of any one of items 1 to 107, wherein R⁵ is a monosubstituted five membered heterocyclyl containing from 1 to 3 heteroatoms selected from the group consisting of O, N and S.
123. The compound of any one of items 1 to 107, wherein R⁵ is a monosubstituted six membered heterocyclyl containing from 1 to 3 heteroatoms selected from the group consisting of O, N and S.
124. The compound of any one of items 1 to 107, wherein R⁵ is a disubstituted five or six membered heterocyclyl containing from 1 to 3 heteroatoms selected from the group consisting of O, N and S.
125. The compound of any one of items 1 to 107, wherein R⁵ is a disubstituted five membered heterocyclyl containing from 1 to 3 heteroatoms selected from the group consisting of O, N and S.
126. The compound of any one of items 1 to 107, wherein R⁵ is a disubstituted six membered heterocyclyl containing from 1 to 3 heteroatoms selected from the group consisting of O, N and S.
127. The compound of any one of items 1 to 107, wherein R⁵ is 2-methoxyphenyl.
128. The compound according to item 1, which is selected from the group consisting of:
(3-(((((1R,4R)-4-((2-Methoxybenzamido)methyl)-4-(thiophen-3-yl)cyclohexyl)oxy)carbonyl)amino)propyl)triphenylphosphonium iodide;
(3-(((((1S,4S)-4-((2-Methoxybenzamido)methyl)-4-(thiophen-3-yl)cyclohexyl)oxy)carbonyl)amino)propyl)triphenylphosphonium iodide;
(3-(((((1R,4R)-4-((2-Methoxybenzamido)methyl)-4-(thiophen-2-yl)cyclohexyl)oxy)carbonyl)amino)propyl)triphenylphosphonium iodide;
(3-(((((1S,4S)-4-((2-Methoxybenzamido)methyl)-4-(thiophen-2-yl)cyclohexyl)oxy)carbonyl)amino)propyl)triphenylphosphonium iodide;
(3-(((((1R,4R)-4-((2-Methoxybenzamido)methyl)-4-phenylcyclohexyl)oxy)carbonyl)amino)propyl)triphenylphosphonium iodide;
(3-(((((1S,4S)-4-((2-methoxybenzamido)methyl)-4-phenylcyclohexyl)oxy)carbonyl)amino)propyl)triphenylphosphonium iodide;
(4-(((((1R,4R)-4-((2-Methoxybenzamido)methyl)-4-(thiophen-3-yl)cyclohexyl)oxy)carbonyl)amino)butyl)triphenylphosphonium iodide;
(4-(((((1S,4S)-4-((2-Methoxybenzamido)methyl)-4-(thiophen-3-yl)cyclohexyl)oxy)carbonyl)amino)butyl)triphenylphosphonium iodide;
(4-(((((1R,4R)-4-((2-Methoxybenzamido)methyl)-4-(thiophen-2-yl)cyclohexyl)oxy)carbonyl)amino)butyl)triphenylphosphonium iodide;
(4-(((((1S,4S)-4-((2-Methoxybenzamido)methyl)-4-(thiophen-2-yl)cyclohexyl)oxy)carbonyl)amino)butyl)triphenylphosphonium iodide;
(4-(((((1R,4R)-4-((2-Methoxybenzamido)methyl)-4-phenylcyclohexyl)oxy)carbonyl)amino)butyl)triphenylphosphonium iodide; and
(4-(((((1S,4S)-4-((2-Methoxybenzamido)methyl)-4-phenylcyclohexyl)oxy)carbonyl)amino)butyl)triphenylphosphonium iodide.
   129. The compound of any one of items 1 to 128 for use in medicine.
   130. The compound of any one of items 1 to 128 for use in the treatment or prevention of a condition, the treatment of which is affected or facilitated by mitochondrial Kv1.3 inhibition, in a warm-blooded animal, such as a mammal (for example, rat, mouse, guinea pig, rabbit, sheep, horse, pig, cow, monkey, human), preferably human.
   131. The compound for use according to item 130, wherein the condition is a cancer.
   132. The compound for use according to item 130, wherein the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, smooth muscle cancer, skeletal muscle cancer, prostate cancer, renal cancer, skin cancer, testicular cancer, cancer and/or tumors of the anus, bile duct cancer, bone cancer, bone marrow cancer, eye cancer, gall bladder cancer, kidney cancer, mouth cancer, laryngeal cancer, esophagus cancer, stomach cancer, cervix cancer, mesothelioma cancer, neuroendocrine cancer, spinal cord, thyroid cancer, vaginal cancer, vulva cancer, uterus cancer, liver cancer, muscle cancer, blood cell cancer (including lymphomas and leukemias).
   133. The compound for use according to item 130, wherein the cancer is selected from the group consisting of breast cancer, colon cancer, prostate cancer, melanoma, smooth muscle cancer, skeletal muscle cancer, chronic lymphocytic leukemia, glioblastoma, and pancreatic ductal adenocarcinoma.
   134. Pharmaceutical composition comprising a compound or any one of items 1 to 128, or a pharmaceutically acceptable salt or hydrate thereof, and a pharmaceutically acceptable excipient and/or carrier.
   135. The pharmaceutical composition according to item 134, further comprising a (further) anticancer agent.
   136. The pharmaceutical composition according to item 135, wherein the anticancer agent is selected from the group consisting of 13-cis-Retinoic Acid, 2-Amino-6-Mercaptopurine, 2-CdA, 2-Chlorodeoxy adenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ. Alkeran, All-transretinoic acid, Alpha interferon, Altretamine, Amethopterin, Amifostine, Aminoglute thimide, Anagrelide, Anandron, AnastroZolc, Arabininosyl cytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabinc liposomal, Cytosar-U. Cytoxan, Dacarbazine, Dactinomycin, Darbepoctin alfa, Daunomycin, Daunorubicin, Daunorubicin hydrochloride, Daunorubicin liposomal, DaunoXome, Decadron, Delta Cortef, Deltasone, Denileukin diftitox, DepoCyt, Dexamethasonc, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, ErbituX, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate. Oncospar. Oncovin, Ontak, Onxal, Oprevelkin, Orapred. Orasone, Oxaliplatin, Paclitaxel, Pamidronate, Panretin, Paraplatin, Peodiapred. PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustineimplant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solumedrol, STI-571, Streptozocin, Tamoxifen, Targretin, Taxol. Taxotere, Temodar, Temozolomide, Teniposide, TESPA, Thalidomide, Thalomid, TheraCys. Thioguanine. Thioguanine Tabloid. Thiophosphoamide. Thioplex. Thiotepa, TICE. Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium Succinate, Hydrocortone phosphate, Hydroxyurea, Ibritumomab, Ibritumomab Tiuxetan, Idamycin, Idarubicin, Ifex, IFN-alpha, Ifosfamide, IL2, IL-11, Imatinib mesylate. Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C. Mitoxantrone, M-Prednisol, MTC, MTX, Mustargcn, Mustine, Mutamycin, Myleran, Iressa, Irinotecan, Isotretinoin, Kidrolasc, Lanacort, L-asparaginase, and LCR.
   137. The pharmaceutical composition according to any one of items 134 to 136 for use in medicine.
   138. The pharmaceutical composition according to any one of items 134 to 136 for use in the treatment or prevention of a condition, the treatment of which is affected or facilitated by mitochondrial Kv1.3 inhibition, in a warm-blooded animal, such as a mammal (for example, rat, mouse, guinea pig, rabbit, sheep, horse, pig, cow, monkey, human), preferably human.
   139. The pharmaceutical composition for use according to item 138, wherein the condition is a cancer.
   140. The pharmaceutical composition for use according to item 139, wherein the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, smooth muscle cancer, skeletal muscle cancer, prostate cancer, renal cancer, skin cancer, testicular cancer, cancer and/or tumors of the anus, bile duct cancer, bone cancer, bone marrow cancer, eye cancer, gall bladder cancer, kidney cancer, mouth cancer, laryngeal cancer, esophagus cancer, stomach cancer, cervix cancer, mesothelioma cancer, neuroendocrine cancer, spinal cord, thyroid cancer, vaginal cancer, vulva cancer, uterus cancer, liver cancer, muscle cancer, blood cell cancer (including lymphomas and leukemias).
   141. The pharmaceutical composition for use according to item 139, wherein the cancer is selected from the group consisting of breast cancer, colon cancer, prostate cancer, melanoma, smooth muscle cancer, skeletal muscle cancer, chronic lymphocytic leukemia, glioblastoma, and pancreatic ductal adenocarcinoma.
   142. A method of inhibiting K_{V}1.3 channels in a warm-blooded animal in need of such treatment, comprising administering to the animal an effective amount of compound of any one of items 1 to 128.
   143. The compound for use of any one of items 130 to 133, the pharmaceutical composition for use of any one of items 138 to 141, or the method of item 142, wherein warm-blooded animal is a mammal.
   144. The compound for use of any one of items 130 to 133, the pharmaceutical composition for use of any one of items 138 to 141, or the method of item 142, wherein warm-blooded animal is a human.
   145. A process for preparing a compound as defined in any one of items 1 to 128 (with the variable groups being as defined in any of items 1 to 127), which process comprises:
      Process step a) transformation of a compound of formula (XI)

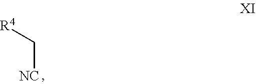

wherein R⁴ is as defined above,
to a compound of formula (XII)

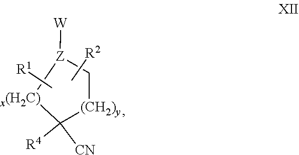

wherein R¹, R², R⁴, x and y are as defined above, Z is selected from CH or N, and W is selected from —H, hydroxyl, —NHR⁸, —COOH, —COO(C1-C6), —CHR⁸, —SO₃H or —SH,
and
Process step b) transformation of a compound of formula (XII)

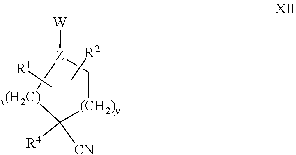

to a compound of formula (XIII)

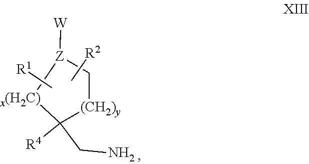

wherein R¹, R², R⁴, Z, W, x and y are as defined above,
and Process step c) transformation of a compound of formula (XIII)

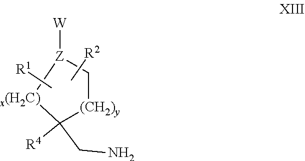

to a compound of formula (XIV)

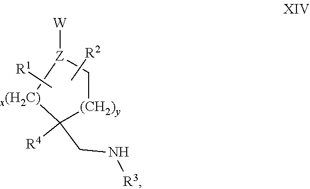

wherein R¹, R², R³, R⁴, Z, W, x and y are as defined above,
and
Process step d) reacting a compound of formula (XIV) with a compound of formula (XV):

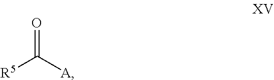

wherein A is selected from hydroxyl, alkoxy, halogen (preferably Cl, Br or I),
to a compound of formula (XVI):

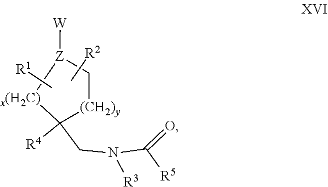

wherein R¹, R², R³, R⁴, R⁵, Z, W, x and y are as defined above,
and
Process step e) reacting a compound of formula (XVI) with a compound of formula (XVII):

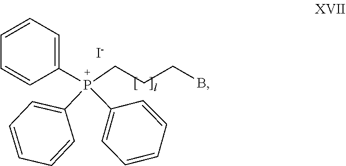

wherein B is selected from hydroxyl, mesyl, tosyl, halogen (preferably I),
to a compound of formula (XVIII):

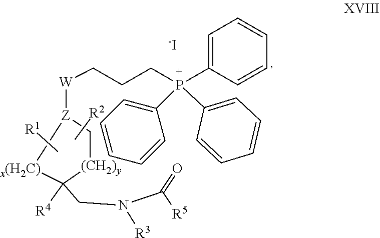

wherein R¹, R², R³, R⁴, R⁵, W, Z, x and y are as defined above.

### BRIEF DESCRIPTION OF THE DRAWINGS

FIG. 1. Cytotoxicity of the indicated compounds. Cells plated on 96-well plates were incubated in the presence of 50 μM of the indicated compounds and fluorescence of the dead-cell marker Cytotox green was monitored over time. In the Figure, fluorescence values after 24 h are reported normalized to the value obtained in the presence of DMSO. All compounds increased cytotoxicity by at least 5-10 times, while 2 and 8 produced over 15-times more cell death than the control. The symbols indicate biological replicates (with three technical replicates each).

FIG. 2. Levels of apoptosis observed in the presence of the indicated compounds after 24 hours incubation. Apoptosis was measured as activation of the executor caspases 3/7, using a fluorescent indicator. The values were normalized against the apoptosis level observed in the presence of the solvent (DMSO). The symbols indicate biological replicates (with three technical replicates each).

FIG. 3. Cytotoxicity of the indicated compounds on tumor spheroids. Fluorescence of the dead-cell marker Cytotox green was monitored over time. In the Figure, fluorescence values after 48 h in the presence of the indicated concentration of the compound are reported normalized to the value obtained in the presence of DMSO. The symbols indicate biological replicates (with three technical replicates each).

FIG. 4. Levels of apoptosis observed in the presence of the indicated compounds during 48 h of treatment. Images were acquired every hour. Apoptosis was measured as activation of the executor caspases 3/7, using a fluorescent indicator. Mean±SEM, three replicates.

FIG. 5. Levels of apoptosis observed in the presence of the indicated compounds over time at two different concentrations (the curves for control and 5 μM are the same as in FIG. 4). Apoptosis was measured as activation of the executor caspases 3/7, using a fluorescent indicator. The open symbols in orange represent 5 μM, solid symbols indicate 25 μM.

FIG. 6. Comparison of the time course of the induction of apoptosis (orange) and of cell death (blue) in the presence of 25 μM compound 8. The vertical line has been included only to indicate the initiation of cell death.

### DETAILED DESCRIPTION OF THE INVENTION

### Definitions

Definitions in this specification and in the claims that follow, reference will be made to a number of terms, which shall be defined to have the following meanings: As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like. When ranges of values are disclosed, and the notation “from nᵢ . . . to n₂” is used, where nᵢ and n₂ are the numbers, then unless otherwise specified, this notation is intended to include the numbers themselves and the range between them. This range can be integral or continuous between and including the end values. By way of example, the range “from 2 to 6 carbons” is intended to include two, three, four, five, and six carbons, since carbons come in integer units. Compare, by way of example, the range “from 1 to 3 μM,” which is intended to include 1 μM, 3 μM, and everything in between to any number of significant figures (e.g., 1.255 μM, 2.1 μM, 2.9999 μM, etc.).

The term “about” as used herein is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value itself and the range which would be included by rounding up or down to that figure as well, taking into account significant figures. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, the terms “treating” and “treatment” refer to delaying the onset of, retarding or reversing the progress of, or alleviating or preventing either the disease or condition to which the term applies, or one or more symptoms of such disease or condition.

The term “individual” (and, equivalently, “subject” or “patient”) means all mammals including humans. Examples of individuals include humans, cows, dogs, cats, goats, sheep, pigs, and rabbits. Preferably, the individual is a human.

The term “disease” as used herein is intended to be generally synonymous, and is used interchangeably with, the terms “disorder,” “syndrome,” and “condition” (as in medical condition), in that all reflect an abnormal condition of an individual (e.g., a human or animal body or of one or more of its parts that impairs normal functioning), is typically manifested by distinguishing signs and symptoms, and/or causes the individual to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

As used herein, the term “administering” means oral administration, administration as a suppository, topical contact, intravenous, intraperitoneal, intramuscular, intralesional, intranasal or subcutaneous administration, or the implantation of a slow-release device, e.g., a miniosmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, and the like.

The term “therapeutically acceptable” refers to those compounds (or salts, tautomers, zwitterionic forms, etc.) which are suitable for use in contact with the tissues of patients without undue toxicity, irritation, and allergic response, are commensurate with a reasonable benefit/risk ratio, and are effective for their intended use. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

As used herein, the term “alkyl”, unless otherwise indicated, includes those alkyl groups of a designated number of carbon atoms of either a straight, branched, or cyclic configuration (carbocycles). Examples of “alkyl” include methyl, ethyl, propyl, isopropyl, butyl, sec-and tert-butyl, pentyl, hexyl, heptyl, cyclopropyl, cyclobutyl, cyclopentyl, cyclohexyl, cycloheptyl, norbornyl, and the like. Preferably alkyl is understood in the context of this invention C₁₋₆ alkyl like methyl, ethyl, propyl, butyl, pentyl, or hexyl; and more preferably is C₁. 4 alkyl like methyl, ethyl, propyl or butyl. Wherein alkyl includes cyclic as well as acyclic groups and is unsubstituted or substituted with one, two or three of the substituents selected from the group consisting of: halo, wherein halo is fluoro, chloro, bromo, or iodo, hydroxy, oxo, O[(C═O)Oᵣ]ₛ(C1-C6)-alkyl, (C1-C6)-alkyl-S(O)ₙ—, aryl-(C1-C6)-alkyloxy, cyano, nitro, vinyl, NR⁶R⁷, O(CO)NR⁶R⁷, CHO, COOH, CO(C1-C6)-alkyl, COO(C1-C6)-alkyl, CONR⁶R⁷, aryl, heteroaryl, heterocyclyl, benzyl-S(O)ₙ—, O[(C═O)Oᵣ]ₛ(C2-C6)alkenyl, O[(C═O)Oᵣ]ₛaryl, O[(C═O)Oᵣ]ₛheteroaryl, O(CH₂)ₙheteroaryl, O(CH₂)ₙaryl, fused benzo, CF₃, ═N—O—(C1-C6)alkyl-COO—(C1-C3)alkyl, S(O)ₙ—(C0-C6)alkyl-aryl, wherein aryl is as defined herein, or S(O)ₙ—(C0-C6)alkyl-heteroaryl, wherein heteroaryl is as defined herein;

“Alkoxy” represents an alkyl group attached through an oxygen bridge, such as methoxy, ethoxy, propoxy, butoxy and pentoxy. The following illustrate the foregoing definitions: “O—(C1-C3)-alkyl” may be methoxy, ethoxy, n-propoxy, i-propoxy, or cyclopropoxy.

“Alkenyl” is intended to include hydrocarbon chains of a specified number of carbon atoms of either a straight- or branched-configuration and at least one unsaturation, which may occur at any point along the chain, such as ethenyl, propenyl, butenyl, pentenyl, dimethyl pentenyl, and the like, and includes E and Z forms, where applicable. Preferably in the context of this invention alkenyl is C₂₋₆ alkenyl like ethylene, propylene, butylene, pentylene, or hexylene; and more preferably is C₂₋₄ alkenyl, like ethylene, propylene, or butylene.

Wherein alkenyl is unsubstituted or substituted with one or two of the substituents selected from the group consisting of: halo, wherein halo is fluoro, chloro, bromo, or iodo, hydroxy, oxo, (C₁-C₆)-alkyloxy, (C₁-C₆)—S(O)ₙ—, phenyl-(C₁-C₆)-alkyloxy, cyano, nitro, vinyl, NR⁶R⁷, NR⁶CO(C₁-C₆)-alkyl, CHO, COOH, CO(C₁-C₆)-alkyl, COOC(C₁-C₆)-alkyl, CONR⁶R⁷, aryl, heteroaryl, heterocyclyl, O[(C═O)Oᵣ]ₛ(C₁-C₆)-alkyl, O[(C═O)Oᵣ]ₛ(C₂-C₆)-alkenyl, O[(C═O)Oᵣ]ₛaryl, O[(C═O)Oᵣ]ₛheteroaryl, O(CH₂)ₙheteroaryl, and O(CH₂)ₙaryl.

“Alkynyl” is intended to include hydrocarbon chains of a specified number of carbon atoms of either a straight- or branched-configuration and at least one unsaturation, which may occur at any point along the chain, such as ethyne, propyne, butyene, pentyne, hexyne, heptyne, or octyne, and the like, and includes E and Z forms, where applicable. Preferably in the context of this invention is alkynyl C₂₋₆ alkynyl like ethyne, propyne, butyene, pentyne, or hexyne; and more preferably is C₂₋₄ alkynyl like ethyne, propyne, butyene, pentyne, or hexyne.

Wherein alkynyl is unsubstituted or substituted with one or two of the substituents selected from the group consisting of: halo, wherein halo is fluoro, chloro, bromo, or iodo, hydroxy, oxo, (C₁-C₆)-alkyloxy, (C₁-C₆)—S(O)ₙ—, phenyl-(C₁-C₆)-alkyloxy, cyano, nitro, vinyl, NR⁶R⁷, NR⁶CO(C₁-C₆)-alkyl, CHO, COOH, CO(C₁-C₆)-alkyl, COOC(C₁-C₆)-alkyl, CONR⁶R⁷, aryl, heteroaryl, heterocyclyl, O[(C═O)Oᵣ]ₛ(C₁-C₆)-alkyl, O[(C═O)Oᵣ]ₛ(C₂-C₆)-alkenyl, O[(C═O)Oᵣ]ₛaryl, O[(C═O)Oᵣ]ₛheteroaryl, O(CH₂)ₙheteroaryl, and O(CH₂)ₙaryl.

Aryl is a partially saturated or unsaturated, mono or bicyclic carbon ring that contains 3-12 atoms; wherein a —CH₂— group can optionally be replaced by a —C(O)—. Particularly aryl is a monocyclic ring containing 5 or 6 atoms or a bicyclic ring containing 9 or 10 atoms. In another aspect aryl is a totally unsaturated ring. Suitable values for aryl include cyclopentenyl, cyclohexenyl, phenyl, naphthyl, indanyl or 1-oxoindanyl. Examples of aryl are optionally substituted phenyl and naphthyl. If substituted, the aryl is substituted with one or more (such as one or two) substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3, preferably n at each occurrence being 0 or 1.

Heterocyclyl is a saturated, partially saturated or unsaturated, optionally substituted monocyclic ring containing 5 to 7 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—, a ring sulphur atom may be optionally oxidised to form the S-oxide(s), and a ring nitrogen atom may be optionally oxidised to form the N-oxide. Examples and suitable values of the term heterocyclyl are morpholino, morpholinyl, piperidino, piperidyl, pyridyl, pyridyl-N-oxide, pyranyl, pyrrolyl, imidazolyl, thiazolyl, thienyl, dioxolanyl, thiadiazolyl, piperazinyl, isothiazolidinyl, triazolyl, tetrazolyl, pyrrolidinyl, 2-oxazolidinonyl, 5-isoxazolonyl, thiomorpholino, pyrrolinyl, homopiperazinyl, 3,5-dioxapiperidinyl, 3-oxopyrazolin-5-yl, tetrahydropyranyl, tetrahydrothiopyranyl, 1-oxotetrahydrothiopyranyl, 1,1-dioxotetrahydrothiopyranyl, pyrimidyl, pyrazinyl, pyridazinyl, pyrazolyl, pyrazolinyl, isoxazolyl, 4-oxopydridyl, 2-oxopyrrolidyl, 4-oxothiazolidyl, furyl, thienyl, oxazolyl, oxadiazolyl, 2-[(5-oxo)-[oxa-3,4-diazolyl] and 3-[oxa-2,4-diazolyl].

Suitably a heterocyclyl is morpholino, morpholinyl, piperidino, piperidyl, pyridyl, pyranyl, pyrrolyl, imidazolyl, thiazolyl, thienyl, thiadiazolyl, piperazinyl, isothiazolidinyl, 1,3,4-triazolyl, tetrazolyl, pyrrolidinyl, thiomorpholino, pyrrolinyl, homopiperazinyl, 3,5-dioxapiperidinyl, pyrimidyl, pyrazinyl, pyridazinyl, pyrazolyl, pyrazolinyl, isoxazolyl, 4-oxopydridyl, 2-oxopyrrolidyl, 4-oxothiazolidyl, furyl, thienyl, oxazolyl, 1,3,4-oxadiazolyl, 1,2,4-oxadiazolyl 2-[(5-oxo)-[oxa-3,4-diazolyl] and 3-[oxa-2,4-diazolyl].

Conveniently heterocyclyl is oxazolyl, 1,3,4-oxadiazolyl, 1,2,4-oxadiazolyl, 2-[(5-oxo)-[oxa-3,4-diazolyl], 3-[oxa-2,4-diazolyl], tetrazolyl, thiazolyl, thiadiazolyl, pyridyl, imidazolyl, furyl, thienyl, morpholine, pyrimidyl, pyrazinyl, pyridazinyl, pyrazolyl, pyrazolinyl, and piperazinyl.

In this context, the prefixes 3-, 4-, 5-, 6-, 7-, 8-, 9- and 10-membered denote the number of ring atoms, or range of ring atoms, whether carbon atoms or heteroatoms. For example, the term “3-10 membered heterocyclyl”, as used herein, pertains to a heterocyclyl group having 3, 4, 5, 6, 7, 8, 9 or 10 ring atoms or a range comprising any of two of those integers. Examples of heterocyclyl groups include 5-6-membered monocyclic heterocyclyls and 9-10 membered fused bicyclic heterocyclyls. Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring). Heterocyclyls also encompass aromatic heterocyclyls and non-aromatic heterocyclyls. Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. “Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to four heteroatoms typically selected from nitrogen, sulfur and oxygen. The heteroaryl ring will contain up to 4 heteroatoms, more typically up to 3 heteroatoms, more usually up to 2, for example a single heteroatom. In one case, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered heteroaryl groups containing nitrogen include pyridyl (1 nitrogen), pyrazinyl, pyrimidinyl and pyridazinyl (2 nitrogens). It will be understood that, such as in the case of pyridyl when substituted with an oxo (═O) substituent the group may be interchangeably referred to as a pyridinone group.

Aromatic heterocyclyl groups may also be bicyclic or polycyclic heteroaromatic ring systems such as fused ring systems (including purinyl, pteridinyl, napthyridinyl, 1H-thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphthyl, indenyl, azulenyl, fluorenyl, anthracenyl and the like, such as 5- or 6-membered aromatic heterocyclyls fused to a phenyl ring including 5-membered aromatic heterocyclyls containing nitrogen fused to a phenyl ring, 5-membered aromatic heterocyclyls containing 1 or 2 nitrogens fused to a phenyl ring and such as 5- or 6-membered aromatic heteroaryls fused to a 6-membered aromatic or non-aromatic heterocyclyls.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; 1) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms. Particular examples of bicyclic heteroaryl groups containing a five membered ring fused to another five membered ringi.e.8-membered fused bicyclic rings include but are not limited to imidazothiazole(e.g.imidazo[2,1-b]thiazole) and imidazoimidazole(e.g.imidazo[1,2-a]imidazole).

Particular examples of bicyclic heteroaryl groups containing a six membered ring fused to a five membered ring, i.e. 9-membered fused bicyclic rings include but are not limited to benzofuran, benzothiophene, benzimidazole, benzoxazole, isobenzoxazole, benzisoxazole, benzothiazole, benzoisothiazole, isobenzofuran, indole, isoindole, indolizine, indoline, isoindoline, purine (e.g. adenine, guanine), indazole, imidazopyridine (e.g. imidazo[1,2-a]pyridine and imidazo[4,5-b]pyridine], pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings i.e. 10-membered fused bicyclic rings include but are not limited to quinoline, isoquinoline, chroman, thiochroman, chromene (including those optionally substituted with oxo (═O) group e.g. oxochromene), isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from N, S and O.

Non-aromatic heterocyclyls may be 3-7 membered mono-cyclic rings.The term “3-7 membered monocyclic”, as used herein, pertains to a mono-cyclic group having 3, 4, 5, 6 or 7 ring atoms or a range comprising any of two of those integers. Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihydropyranyl, tetrahydropyranyl, 2H-pyranyl, 4H-pyranyl, thianyl, thianyl oxide, thianyl dioxide, piperazinyl, dioxanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like. Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, naphthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like. The term “spiro ring system” means a bicyclic ring system in which the rings are connected via a single shared atom or “spiroatom” more particularly a quaternary carbon (“spiro carbon”) and encompasses spiro bicyclic 7-11-membered carbocyclic rings and spiro bicyclic 7-11-membered heterocyclic rings containing one, two, three or four heteroatoms independently selected from O, N and S.

Examples of heterocyclyl-C₁₋₄ alkyl are morpholinomethyl, morpholinoethyl, morpholinylmethyl, morpholinylethyl, piperidinomethyl, piperidinoethyl, piperidylmethyl, piperidylethyl, imidazolylmethyl, imidazolylethyl, tetrazolylmethyl, tetrazolylethyl, oxazolylmethyl, oxazolylethyl, 1,3,4-oxadiazolylmethyl, 1,2,4-oxadiazolylmethyl, 1,2,4-oxadiazolylethyl, pyridylmethyl, pyridylethyl, furylmethyl, furylethyl, (thienyl)methyl, (thienyl)ethyl, pyrazinylmethyl, pyrazinylethyl, piperazinylmethyl and piperazinylethyl.

If substituted, the heterocyclyl, such as heteroaryl, is substituted with one or more (such as one, two or three) substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3, preferably n at each occurrence being 0 or 1.

In the compounds of Formula I, the aryl or heterocyclyl groups may be optionally substituted with the substituents listed above at any available carbon atom or nitrogen atom (if present), but compounds bearing certain substitutents, directly substituted to a nitrogen may be relatively unstable and are not preferred. The heteroaryl may, for example, also be fused to a second 5-, 6-, or 7-membered ring containing one or two oxygens such as: dioxolanyl, dihydrofuranyl, dihydropyranyl, and dioxanyl. Disubstituted aryl groups may be ortho, para or meta and all three are intended unless specifically defined otherwise.

The term “halogen” is used to denote fluoro, chloro, bromo, or iodo. Particular halogens are chloro and bromo. More particular halogen is chloro.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are the hydrochloride salts, methanesulfonic acid salts and citric acid salts.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following Derivatives of the present compounds: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (April 2002).

The term “warm-blooded animal” refers to a member of the animal kingdom which possesses a homeostatic mechanism and includes mammals and birds.

### Compound of the Present Invention

The present invention provide is a main aspect compounds of formula (I) which are potassium channel inhibitors.

More specifically, the present invention provides a compound of formula (I):

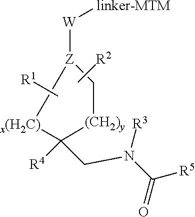

wherein:
“MTM” is a mitochondria targeting moiety;
x and y are independently 0, 1, or 2;
Z is CH or N;
R¹ and R² are independently selected from the group consisting of hydrogen, halo, wherein halo is fluoro, chloro, bromo, or iodo, hydroxy, HO(C₁-C₆)-alkyloxy, (C₁-C₄)-perfluoroalkyl, O(CO)CCl₃, (C₁-C₆)-alkyl-S(O)ₙ—, phenyl-(CH₂)ᵣ—S(O)ₙ—, cyano, nitro, COOH, CO(C₁-C₆)-alkyl, COO(C₁-C₆)-alkyl, CONR⁶R⁷, NR⁶R⁷, O(CO)NR⁶R⁷, azido, NR⁶(CO)NR⁶R⁷, (C₁-C₁₀)-alkyl, (C₂-C₁₀)-alkenyl, (C₂-C₁₀)-alkynyl, O[(C═O)Oᵣ]ₛ(C₁-C₆)-alkyl, O[(C═O)Oᵣ]ₛ(C₂-C₆)-alkenyl, O[(C═O)Oᵣ]ₛaryl, O[(C═O)Oᵣ]ₛheteroaryl, O(CH₂)ₙheteroaryl, aryl, O(CH₂)ₙaryl, oxo, ═CH—(C₁-C₆)-alkyl, ═CH—(C₂-C₆)-alkenyl, ═CH-aryl, and ═CH₂, with r and s at each occurrence being independently from each other 0 or 1, and n at each occurrence being 0, 1, 2 or 3, preferably n at each occurrence being 0 or 1;
R³ is hydrogen, [(C═O)Oᵣ]ₛaryl or [(C═O)Oᵣ]ₛ(C₁-C₆)-alkyl, with r and s at each occurrence being independently from each other 0 or 1;
R⁴ and R⁵ are independently selected from the group consisting of substituted or unsubstituted aryl, such as phenyl or naphtyl, and substituted or unsubstituted five or six membered heterocyclyl containing from 1 to 3 heteroatoms selected from the group consisting of O, N and S;
wherein the aryl, if substituted, is substituted with one or more (such as one or two) substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3, preferably n at each occurrence being 0 or 1;
wherein the heterocyclyl, if substituted, is substituted with one or more (such as one or two) substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3, preferably n at each occurrence being 0 or 1;
R⁶ and R⁷ are independently selected from the group consisting of hydrogen, [(C═O)Oᵣ]ₛaryl, [(C═O)Oᵣ]ₛ(C₂-C₈)-alkenyl, [(C═O)Oᵣ]ₛ(C₁-C₈)-alkyl, (C═O)ᵣS(O)ₙ(C₁-C₈)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1;
W is a suitable functional group depending on the available site on the particular K_{V}1.3 inhibitor of interest which is attached to the linker;

Linker is selected from:
—(C(R⁹)(R¹⁰))_{I}—, wherein I is from 1 to 20, preferably from 1 to 10, more preferably 3 to 5; and R⁹ and R¹⁰ are independently of one another —H, halogen, —CF₃, —OH, —(C₁-C₆)-alkyl, —OC(O)(C₁-C₆)-alkyl, [(C═O)Oᵣ]ₛaryl, [(C═O)Oᵣ]ₛheteroaryl, or [(C═O)Oᵣ]ₛ(C₁-C₆)-alkyl, with r and s at each occurrence being independently from each other 0 or 1;
Non-peptidic polymeric linkers, such as non-peptidic polymeric linkers selected from polyalkylene oxides (e.g. polyethylene glycol, polypropylene glycol, and the like), polyvinyl alcohol, polyvinylpyrrolidone as well as derivatives and copolymers thereof;
Non-polymeric aliphatic linkers, such as non-polymeric aliphatic linkers comprising a divalent, linear or branched, straight or cyclic, saturated or unsaturated hydrocarbon chain having from 2 to 20 carbon atoms, wherein the carbon atoms are optionally replaced by a group selected from —O—, —S—, —NH—, —C(═O)—, —OC(═O)—, —N(C₁-C₆ alkyl)-, NHC(═O)—, —N(C₁-C₆ alkyl)C(═O)—, —S(═O)— or —S(═O)₂— and wherein the chain is optionally substituted on carbon with one or more (e.g. 1, 2, 3 or 4) substituents;
A divalent radical formed from an amino acid or peptide; and

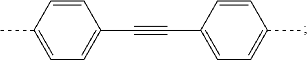

or a pharmaceutically acceptable salt, racemate, diastereomer, enantiomer, ester, carbamate, sulphate, phosphate or prodrug thereof.

Generally, a mitochondria targeting moiety (“MTM”), as disclosed herein, is a moiety that targets the mitochondria by selectively delivering the compound to or accumulating the compound in the mitochondria. Exemplary mitochondria targeting moieties (“MTM”) that can be incorporated into the disclosed compounds are delocalized lipophilic cations, which are effective at crossing the hydrophobic membranes and accumulating in the mitochondria. Any suitable MTM (mitochondria targeting moiety) may be employed in the present invention.

Targeting a drug to mitochondria—or for that matter to any subcellular compartment—can rely on two strategies: a) attaching an “address” moiety to the Kv1.3 active part or b) arranging for transportation by a nanostructured targeted carrier. Within the first approach a distinction can be made between molecules in which the targeting moiety is attached permanently and molecules based on a labile linker, whose splitting will regenerate the parent active portion of the Kv1.3 moiety. Chemical modification entails new pharmacologically relevant properties which need to be taken into consideration. Moderate lipophilicity and molecular weight are required for an optimal membrane permeation. In most cases mitochondrial targeting relies on the transmembrane potential to drive drugs engineered to carry—stably or temporarily—a positive charge into the matrix or mitochondria. In order for the cation to cross biomembranes, in the absence of a specific carrier, the positive charge needs to be delocalized and the molecule as a whole needs to be sufficiently lipophilic. This very often translates into the incorporation into the mitochondriotropic molecule of a triphenylphosphonium (TPP) group connected to the pharmacologically active Kv1.3 moiety via a linker.

Alternative mitochondria targeting groups can be used including dequalinium (DQA), imidazolium, guanidinium, pyridinium, rhodamine, and triethylammonium groups. DQA is a dicationic lipophilic compound formed by two quinaldinium rings linked by ten methylene groups. It can self-assemble into vesicle-like liposomes referred to as DQAsomes, which have been used to deliver chemotherapeutics drugs and genetic material to mitochondria. Imidazolium cations have been used to convey fluorophores to the mitochondria of cultured cells, and could be exploited, in principle, to target pharmaceuticals as well. Conjugation of porphyrins with guanidinium/biguanidinium determined a “clean” mitochondrial localization in cultured cells. Both Rhodamine 12 and Rhodamine 19 are mitochondria-targeting moieties because of their delocalized positive charge and ability to cross biomembranes. Rhodamine 19 has been tested in substitution of TPP to form a mitochondriotropic rhodamine 19-plastoquinone conjugate. Pyridinium has been used as the targeting group, which acts as anticancer mitochondrial uncoupler. Non-cationic compounds can also serve to target and accumulate the disclosed compounds in the mitochondria matrix.Peptides can also be used as mitochondria-targeting devices. These belong to the family of cell-penetrating peptides: positively charged amino acid sequences capable of entering the cell and, at least in principle, to carry along a “cargo” as well. The best-performing Mitochondria Penetrating Peptides alternate charged and lipophilic residues. For example, Szeto-Schiller peptides can serve as suitable mitochondria targeting moieties in the disclosed compounds to target and accumulate the inhibitor in the mitochondria matrix. Any suitable Szeto-Schiller peptide can be used in the disclosed compounds. Still further examples of a mitochondria targeting moiety that can be used herein are cyanine dyes and anthracyclines.

According to some embodiments, MTM is a mitochondria targeting moiety selected from:

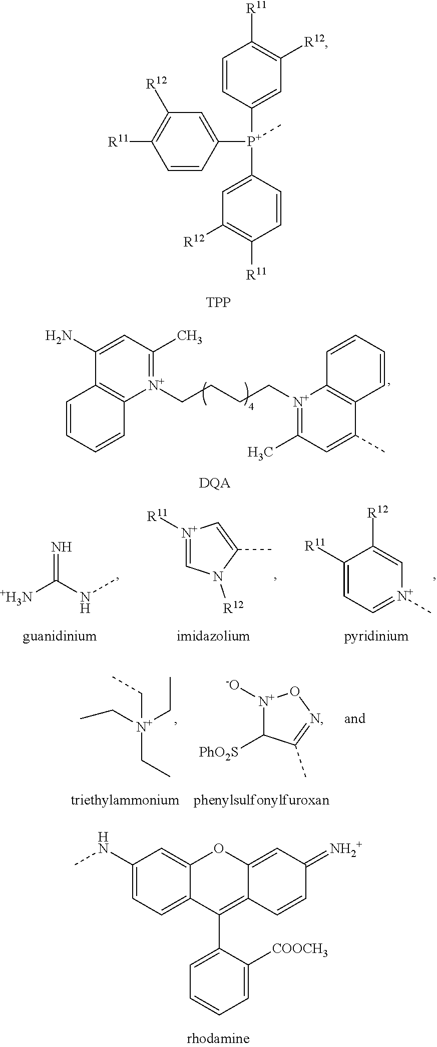

wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

According to some embodiments, MTM is

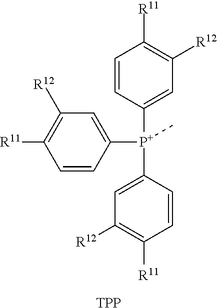

wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

According to some embodiments, MTM is

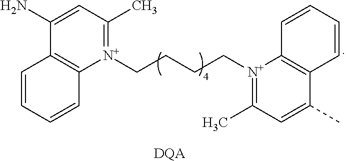

According to some embodiments, MTM is

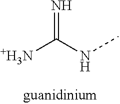

According to some embodiments, MTM is

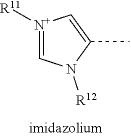

wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

According to some embodiments, MTM is

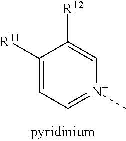

wherein R¹¹ and R¹² are independently of one another —H, halogen, —CF₃, —OH, —(C₁-C₅)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

According to some embodiments, MTM is

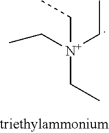

According to some embodiments, MTM is

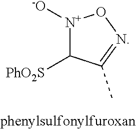

According to some embodiments, MTM is

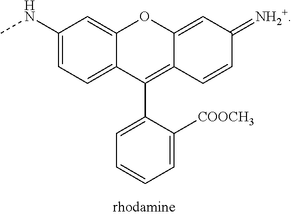

According to some embodiments, R¹¹ and R¹² are each —H.

According to some embodiments, R¹¹ and R¹² are each halogen.

According to some embodiments, R¹¹ and R¹² are each —CF₃.

According to some embodiments, W comprises a cleavable group.

A cleavable group can provide controllable release of the Kv1.3 moiety. Any suitable cleavable group can be employed. Examples of suitable cleavable groups include esters, carbamates, disulfide linkers, oxime linkers, hydrazine groups, diazolinkers, carbonyloxyethylsulfone groups, amino acid groups, phenylacetamide groups, and the like.

According to some embodiments, W comprises a cleavable group selected from esters, carbamates, disulfide linkers, oxime linkers, hydrazine groups, diazolinkers, carbonyloxyethylsulfone groups, amino acid groups, and phenylacetamide groups.

According to some embodiments, W is selected from (C₁-C₆)-alkyl, (C₂-C₆)-alkenyl, (C₂-C₆)-alkynyl, (C₃-C₆)cycloalkyl, aryl, heteroaryl, —OC(O)NR⁸—, —COO—, —OC(O)—, —CONR⁸—, —NHR⁸—, —SO—, —SO₂NR⁸—, —CHR⁸—, —SO₂—, —CO—, —S—, —O—, —CH₂—, —OC(O)—CH₂—C(O)O—, —CH(OH)—CH(OH)—; wherein R⁸ is —H, —F, —Cl, —Br, —OH, —(C₁-C₆)-alkyl, or —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

According to some embodiments, W is (C₁-C₆)-alkyl.

According to some embodiments, W is —OC(O)NR⁸—.

According to some embodiments, W is —CHR⁸—.

According to some embodiments, R⁸ is —H.

According to some embodiments, R⁸ is —(C₁-C₆)-alkyl.

According to some embodiments, R⁸ is halogen.

According to some embodiments, Linker is —(C(R⁹)(R¹⁰))_{I}—, wherein I is from 1 to 20, preferably from 1 to 10,more preferably from 3 to 5; and R⁹ and R¹⁰ are independently of one another —H, halogen, —CF₃, —OH, —(C₁-C₆)-alkyl, —OC(O)(C₁-C₆)-alkyl, [(C═O)Oᵣ]ₛaryl, [(C═O)Oᵣ]ₛheteroaryl or [(C═O)Oᵣ]ₛ(C₁-C₆)-alkyl, with r and s at each occurrence being independently from each other 0 or 1.

According to some embodiments, Linker is a non-peptidic polymeric linker, such as non-peptidic polymeric linker selected from polyalkylene oxides (e.g. polyethylene glycol, polypropylene glycol, and the like), polyvinyl alcohol, polyvinylpyrrolidone as well as derivatives and copolymers thereof.

According to some embodiments, the non-peptidic polymeric linker is polyalkylene oxide, preferably polyethylene glycol or polypropylene glycol.

The polyethylene glycol chain may comprises from 2 to 20 repeating ethylene glycol units. One or both terminal hydroxy groups on the polyethylene glycol chain may be substituted with groups selected from amine, thiol, azide, carboxy, hydroxyl, N-hydroxysuccinimide and maleimide.

The polypropylene glycol chain may comprises from 2 to 20 repeating propylene glycol units. One or both terminal hydroxy groups on the polypropylene glycol chain may be substituted with groups selected from amine, thiol, azide, carboxy, hydroxyl, N-hydroxysuccinimide and maleimide.

According to some embodiments, Linker is a non-polymeric aliphatic linker, such as a non-polymeric aliphatic linker comprising a divalent, linear or branched, straight or cyclic, saturated or unsaturated hydrocarbon chain having from 2 to 20 carbon atoms, wherein the carbon atoms are optionally replaced by a group selected from —O—, —S—, —NH—, —C(═O)—, —OC(═O)—, —N(C₁-C₆ alkyl)-, NHC(═O)—, —N(C₁-C₆ alkyl)C(═O)—, —S(═O)— or —S(═O)₂— and wherein the chain is optionally substituted on carbon with one or more (e.g. 1, 2, 3 or 4) substituents.

The non-polymeric aliphatic linkers are typically derived from an aliphatic compound having at least two functional groups, capable of reacting with functional groups on the Kv1.3 inhibiting moieties (e.g. carboxy, NH₂, OH, and the like).

According to some embodiments, Linker is a divalent radical formed from an amino acid or peptide.

According to some embodiments, Linker is

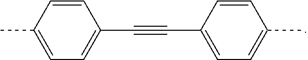

According to some embodiments, Z is CH.

According to some embodiments, Z is N.

According to some embodiments, the compound of formula I is a compound of structural Formula II or Formula III

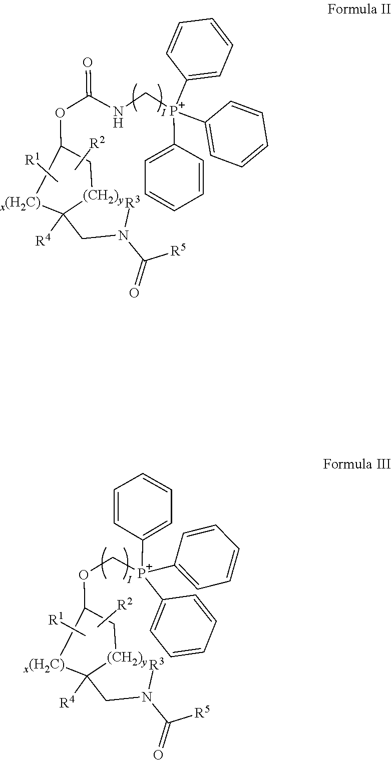

with x, y, I, R¹, R², R³, R⁴ and R⁵ being as defined herein.

According to some embodiments, the compound of formula I is a compound of structural Formula IV or V

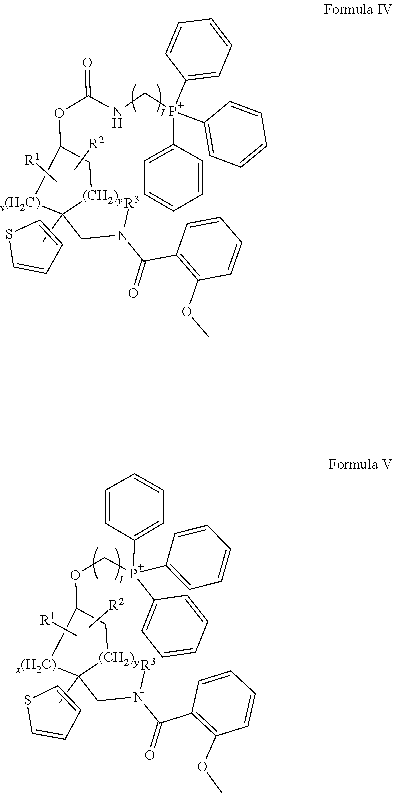

with x, y, I, R¹, R² and R³ being as defined herein.

According to some embodiments, the compound of formula I is a compound of structural Formula VI or Formula VII

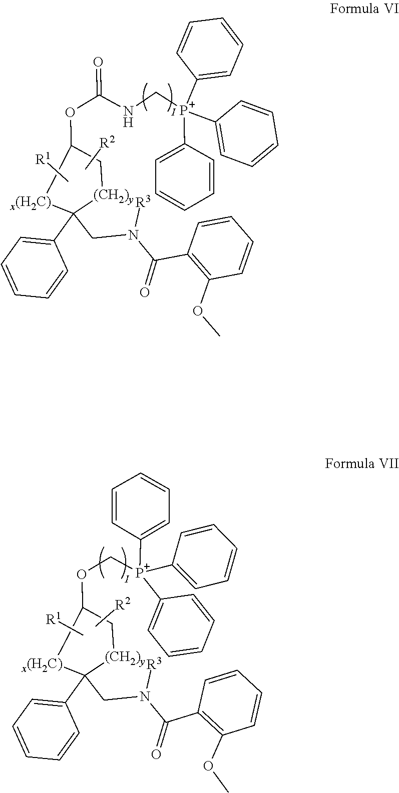

with x, y, I, R¹, R² and R³ being as defined herein.

According to some embodiments, the compound of formula I is a compound of structural Formula VIII or Formula IX

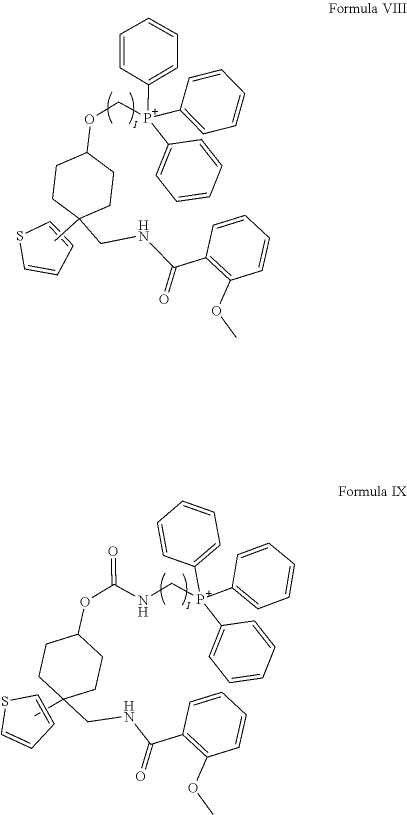

with I being as defined herein.

According to some embodiments, the compound of formula I is an enantiomerically pure compound or an enantiomerically enriched compound with the following structural Formula X or Formula XI:

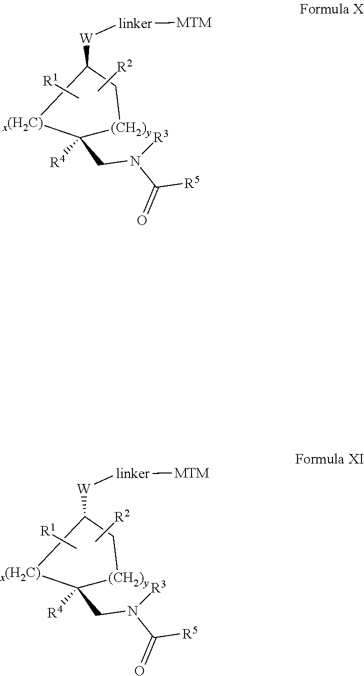

with x, y, I, W, R¹, R², R³, R⁴, R⁵, linker and MTM being as defined herein.

Unless otherwise stated, variables in the embodiments below are defined as for formula (I) and any one of formulae (II to XI), where any such variable is occurring.

According to some embodiments, x is 0.

According to some embodiments, x is 1.

According to some embodiments, x is 2.

According to some embodiments, y is 0.

According to some embodiments, y is 1.

According to some embodiments, y is 2.

According to some embodiments, R¹ is hydrogen.

According to some embodiments, R¹ is halo, preferably wherein halo is fluoro, chloro, bromo, or iodo.

According to some embodiments, R¹ is hydroxy.

According to some embodiments, R¹ is selected from —O(C1-C6)-alkyl.

According to some embodiments, R¹ is selected from —O(CO)NR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurence being independently from each other 0 or 1, and n at each occurrence being 0, 1, 2 or 3, preferably n at each occurrence being 0 or 1.

According to some embodiments, R¹ is selected from —CO(C1-C6)-alkyl.

According to some embodiments, R¹ is selected from COO(C1-C6)-alkyl.

According to some embodiments, R¹ is selected from —CONR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R¹ is carboxyl.

According to some embodiments, R¹ is cyano.

According to some embodiments, R¹ is nitro.

According to some embodiments, R¹ is selected from —NR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R¹ is selected from —NR⁷(CO)NR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R¹ is selected from (C1-C4)-perfluoroalkyl.

According to some embodiments, R¹ is selected from O(CO)CCl₃.

According to some embodiments, R¹ is selected from (C1-C6)-alkyl-S(O)ₙ—.

According to some embodiments, R¹ is selected from phenyl-(CH₂)ᵣ—S(O)ₙ—.

According to some embodiments, R¹ is azido.

According to some embodiments, R¹ is selected from (C1-C10)-alkyl.

According to some embodiments, R¹ is selected from (C2-C10)-alkenyl.

According to some embodiments, R¹ is selected from (C2-C10)-alkynyl.

According to some embodiments, R¹ is selected from O[(C═O)Oᵣ]ₛ(C1-C6)-alkyl.

According to some embodiments, R¹ is selected from O[(C═O)Oᵣ]ₛ(C2-C6)-alkenyl.

According to some embodiments, R¹ is selected from O[(C═O)Oᵣ]ₛaryl.

According to some embodiments, R¹ is selected from O[(C═O)Oᵣ]ₛheteroaryl.

According to some embodiments, R¹ is selected from O(CH₂)ₙheteroaryl.

According to some embodiments, R¹ is aryl.

According to some embodiments, R¹ is selected from O(CH₂)ₙaryl.

According to some embodiments, R¹ is oxo.

According to some embodiments, R¹ is selected from ═CH—(C1-C6)-alkyl.

According to some embodiments, R¹ is selected from ═CH—(C2-C6)-alkenyl.

According to some embodiments, R¹ is selected from ═CH-aryl.

According to some embodiments, R¹ is selected from ═CH₂.

According to some embodiments, R² is hydrogen.

According to some embodiments, R² is halo, preferably wherein halo is fluoro, chloro, bromo, or iodo.

According to some embodiments, R² is hydroxy.

According to some embodiments, R² is —O(C1-C6)-alkyl.

According to some embodiments, R² is selected from —O(CO)NR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R² is selected from —CO(C1-C6)-alkyl.

According to some embodiments, R² is selected from COO(C1-C6)-alkyl.

According to some embodiments, R² is selected from —CONR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R² is carboxyl.

According to some embodiments, R² is cyano.

According to some embodiments, R² is nitro.

According to some embodiments, R² is selected from —NR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R² is selected from —NR⁶(CO)NR⁶R⁷, wherein R⁶ and R⁷ are each independently selected from the group consisting of: hydrogen, [(C═O)Oᵣ]ₛaryl, [(C═O)Oᵣ]ₛ(C2-C8)-alkenyl, [(C═O)Oᵣ]ₛ(C1-C8)-alkyl, (C═O)ᵣS(O)ₙ(C1-C8)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R² is selected from (C1-C4)-perfluoroalkyl.

According to some embodiments, R² is O(CO)CCl₃.

According to some embodiments, R² is selected from (C1-C6)-alkyl-S(O)ₙ—.

According to some embodiments, R² is selected from phenyl-(CH₂)ᵣ—S(O)ₙ—.

According to some embodiments, R² is azido.

According to some embodiments, R² is selected from (C1-C10)-alkyl.

According to some embodiments, R² is selected from (C2-C10)-alkenyl.

According to some embodiments, R² is selected from (C2-C10)-alkynyl.

According to some embodiments, R² is selected from O[(C═O)Oᵣ]ₛ(C1-C6)-alkyl.

According to some embodiments, R² is selected from O[(C═O)Oᵣ]ₛ(C2-C6)-alkenyl.

According to some embodiments, R² is selected from O[(C═O)Oᵣ]ₛaryl.

According to some embodiments, R² is selected from O[(C═O)Oᵣ]ₛheteroaryl.

According to some embodiments, R² is selected from O(CH₂)ₙheteroaryl.

According to some embodiments, R² is aryl.

According to some embodiments, R² is selected from O(CH₂)ₙaryl.

According to some embodiments, R² is oxo.

According to some embodiments, R² is selected from ═CH—(C1-C6)-alkyl.

According to some embodiments, R² is selected from ═CH—(C2-C6)-alkenyl.

According to some embodiments, R² is selected from ═CH-aryl.

According to some embodiments, R² is selected from ═CH₂.

According to some embodiments, R³ is hydrogen.

According to some embodiments, R³ is selected from [(C═O)Oᵣ]ₛaryl.

According to some embodiments, R³ is selected from [(C═O)Oᵣ]ₛ(C1-C6)-alkyl.

According to some embodiments, R⁴ is a substituted or unsubstituted aryl.

According to some embodiments, the aryl is phenyl.

According to some embodiments, the aryl is naphthyl.

According to some embodiments, the aryl is unsubstituted.

According to some embodiments, the aryl is monosubstituted.

According to some embodiments, the aryl is disubstituted.

According to some embodiments, R⁴ is a substituted or unsubstituted five or six membered heterocyclyl containing from 1 to 3 heteroatoms selected from the group consisting of O, N and S.

According to some embodiments, R⁴ is a substituted or unsubstituted five membered heterocyclyl containing from 1 to 3 heteroatoms selected from the group consisting of O, N and S.

According to some embodiments, R⁴ is a substituted or unsubstituted six membered heterocyclyl containing from 1 to 3 heteroatoms selected from the group consisting of O, N and S.

According to some embodiments, R⁴ is an unsubstituted five or six membered heterocyclyl containing from 1 to 3 heteroatoms selected from the group consisting of O, N and S.

According to some embodiments, R⁴ is an unsubstituted five membered heterocycle containing from 1 to 3 heteroatoms selected from the group consisting of O, N and S.

According to some embodiments, R⁴ is an unsubstituted six membered heterocyclyl containing from 1 to 3 heteroatoms selected from the group consisting of O, N and S.

According to some embodiments, R⁴ is a substituted five or six membered heterocyclyl containing from 1 to 3 heteroatoms selected from the group consisting of O, N and S.

According to some embodiments, R⁴ is a monosubstituted five or six membered heterocyclyl containing from 1 to 3 heteroatoms selected from the group consisting of O, N and S.

According to some embodiments, R⁴ is a monosubstituted five membered heterocyclyl containing from 1 to 3 heteroatoms selected from the group consisting of O, N and S.

According to some embodiments, R⁴ is a monosubstituted six membered heterocyclyl containing from 1 to 3 heteroatoms selected from the group consisting of O, N and S.

According to some embodiments, R⁴ is a disubstituted five or six membered heterocyclyl containing from 1 to 3 heteroatoms selected from the group consisting of O, N and S.

According to some embodiments, R⁴ is a disubstituted five membered heterocyclyl containing from 1 to 3 heteroatoms selected from the group consisting of O, N and S.

According to some embodiments, R⁴ is a disubstituted six membered heterocyclyl containing from 1 to 3 heteroatoms selected from the group consisting of O, N and S.

According to some embodiments, R⁴ is a substituted or unsubstituted five or six membered aromatic heterocyclyl containing from 1 to 3 heteroatoms selected from the group consisting of O, N and S.

According to some embodiments, R⁴ is a substituted or unsubstituted five membered aromatic heterocyclyl containing from 1 to 3 heteroatoms selected from the group consisting of O, N and S.

According to some embodiments, R⁴ is a substituted or unsubstituted six membered aromatic heterocyclyl containing from 1 to 3 heteroatoms selected from the group consisting of O, N and S.

According to some embodiments, R⁴ is an unsubstituted five or six membered aromatic heterocyclyl containing from 1 to 3 heteroatoms selected from the group consisting of O, N and S.

According to some embodiments, R⁴ is an unsubstituted five membered aromatic heterocycle containing from 1 to 3 heteroatoms selected from the group consisting of O, N and S.

According to some embodiments, R⁴ is an unsubstituted six membered aromatic heterocyclyl containing from 1 to 3 heteroatoms selected from the group consisting of O, N and S.

According to some embodiments, R⁴ is a substituted five or six membered aromatic heterocyclyl containing from 1 to 3 heteroatoms selected from the group consisting of O, N and S.

According to some embodiments, R⁴ is a monosubstituted five or six membered aromatic heterocyclyl containing from 1 to 3 heteroatoms selected from the group consisting of O, N and S.

According to some embodiments, R⁴ is a monosubstituted five membered aromatic heterocyclyl containing from 1 to 3 heteroatoms selected from the group consisting of O, N and S.

According to some embodiments, R⁴ is a monosubstituted six membered aromatic heterocyclyl containing from 1 to 3 heteroatoms selected from the group consisting of O, N and S.

According to some embodiments, R⁴ is a disubstituted five or six membered aromatic heterocyclyl containing from 1 to 3 heteroatoms selected from the group consisting of O, N and S.

According to some embodiments, R⁴ is a disubstituted five membered aromatic heterocyclyl containing from 1 to 3 heteroatoms selected from the group consisting of O, N and S.

According to some embodiments, R⁴ is a disubstituted six membered aromatic heterocyclyl containing from 1 to 3 heteroatoms selected from the group consisting of O, N and S.

According to some embodiments, R⁴ is an unsubstituted, monosubstituted, or disubstituted thiophene.

According to some embodiments, R⁴ is a 2- or 3-substituted thiophene.

According to some embodiments, R⁵ is a substituted or unsubstituted aryl.

According to some embodiments, the aryl is phenyl.

According to some embodiments, the aryl is naphthyl.

According to some embodiments, the aryl is unsubstituted.

According to some embodiments, the aryl is monosubstituted.

According to some embodiments, the aryl is disubstituted.

According to some embodiments, R⁵ is a substituted or unsubstituted five or six membered heterocyclyl containing from 1 to 3 heteroatoms selected from the group consisting of O, N and S.

According to some embodiments, R⁵ is a substituted or unsubstituted five membered heterocyclyl containing from 1 to 3 heteroatoms selected from the group consisting of O, N and S.

According to some embodiments, R⁵ is a substituted or unsubstituted six membered heterocyclyl containing from 1 to 3 heteroatoms selected from the group consisting of O, N and S.

According to some embodiments, R⁵ is an unsubstituted five or six membered heterocyclyl containing from 1 to 3 heteroatoms selected from the group consisting of O, N and S.

According to some embodiments, R⁵ is an unsubstituted five membered heterocycle containing from 1 to 3 heteroatoms selected from the group consisting of O, N and S.

According to some embodiments, R⁵ is an unsubstituted six membered heterocyclyl containing from 1 to 3 heteroatoms selected from the group consisting of O, N and S.

According to some embodiments, R⁵ is a substituted five or six membered heterocyclyl containing from 1 to 3 heteroatoms selected from the group consisting of O, N and S.

According to some embodiments, R⁵ is a monosubstituted five or six membered heterocyclyl containing from 1 to 3 heteroatoms selected from the group consisting of O, N and S.

According to some embodiments, R⁵ is a monosubstituted five membered heterocyclyl containing from 1 to 3 heteroatoms selected from the group consisting of O, N and S.

According to some embodiments, R⁵ is a monosubstituted six membered heterocyclyl containing from 1 to 3 heteroatoms selected from the group consisting of O, N and S.

According to some embodiments, R⁵ is a disubstituted five or six membered heterocyclyl containing from 1 to 3 heteroatoms selected from the group consisting of O, N and S.

According to some embodiments, R⁵ is a disubstituted five membered heterocyclyl containing from 1 to 3 heteroatoms selected from the group consisting of O, N and S.

According to some embodiments, R⁵ is a disubstituted six membered heterocyclyl containing from 1 to 3 heteroatoms selected from the group consisting of O, N and S.

According to some embodiments, R⁵ is a substituted or unsubstituted five or six membered aromatic heterocyclyl containing from 1 to 3 heteroatoms selected from the group consisting of O, N and S.

According to some embodiments, R⁵ is a substituted or unsubstituted five membered aromatic heterocyclyl containing from 1 to 3 heteroatoms selected from the group consisting of O, N and S.

According to some embodiments, R⁵ is a substituted or unsubstituted six membered aromatic heterocyclyl containing from 1 to 3 heteroatoms selected from the group consisting of O, N and S.

According to some embodiments, R⁵ is an unsubstituted five or six membered aromatic heterocyclyl containing from 1 to 3 heteroatoms selected from the group consisting of O, N and S.

According to some embodiments, R⁵ is an unsubstituted five membered aromatic heterocyclyl containing from 1 to 3 heteroatoms selected from the group consisting of O, N and S.

According to some embodiments, R⁵ is an unsubstituted six membered aromatic heterocyclyl containing from 1 to 3 heteroatoms selected from the group consisting of O, N and S.

According to some embodiments, R⁵ is a substituted five or six membered aromatic heterocyclyl containing from 1 to 3 heteroatoms selected from the group consisting of O, N and S.

According to some embodiments, R⁵ is a monosubstituted five or six membered aromatic heterocyclyl containing from 1 to 3 heteroatoms selected from the group consisting of O, N and S.

According to some embodiments, R⁵ is a monosubstituted five membered aromatic heterocyclyl containing from 1 to 3 heteroatoms selected from the group consisting of O, N and S.

According to some embodiments, R⁵ is a monosubstituted six membered aromatic heterocyclyl containing from 1 to 3 heteroatoms selected from the group consisting of O, N and S.

According to some embodiments, R⁵ is a disubstituted five or six membered aromatic heterocyclyl containing from 1 to 3 heteroatoms selected from the group consisting of O, N and S.

According to some embodiments, R⁵ is a disubstituted five membered aromatic heterocyclyl containing from 1 to 3 heteroatoms selected from the group consisting of O, N and S.

According to some embodiments, R⁵ is a disubstituted six membered aromatic heterocyclyl containing from 1 to 3 heteroatoms selected from the group consisting of O, N and S.

According to some embodiments, R⁵ is 2-methoxyphenyl.

According to some embodiments, x is 2, y is 1, R⁴ is unsubstituted, monosubstituted, or disubstituted thiophene.

According to some embodiments, x is 2, y is 1, R⁴ is 2- or 3-substituted thiophene.

According to some embodiments, x is 2, y is 1, R³ is hydrogen, R⁴ is unsubstituted, monosubstituted, or disubstituted thiophene and R⁵ is 2-methoxyphenyl.

According to some embodiments, x is 2, y is 1, R³ is hydrogen, R⁴ is 2- or 3-substituted thiophene and R⁵ is 2-methoxyphenyl.

According to some embodiments, x is 2, y is 1, R³ is hydrogen, R⁴ is unsubstituted, monosubstituted, or disubstituted thiophene, and R⁵ is 2-methoxyphenyl.

According to some embodiments, x is 2, y is 1, R³ is hydrogen, R⁴ is 2- or 3-substituted thiophene, and R⁵ is 2-methoxyphenyl.

According to some embodiments, R⁶ and R⁷ are independently selected from the group consisting of hydrogen, [(C═O)Oᵣ]ₛaryl, [(C═O)Oᵣ]ₛ(C₂-C₈)-alkenyl, [(C═O)Oᵣ]ₛ(C₁-C₈)-alkyl, (C═O)ᵣS(O)ₙ(C₁-C₈)-alkyl, (C═O)ᵣS(O)ₙaryl, and heterocyclyl, with r and s at each occurrence being independently from each other 0 or 1, and n at each occurrence being 0, 1, 2 or 3, preferably n at each occurrence being 0 or 1.

According to some embodiments, R⁶ is hydrogen.

According to some embodiments, R⁶ is [(C═O)Oᵣ]ₛaryl.

According to some embodiments, R⁶ is [(C═O)Oᵣ]ₛ(C₁-C₈)-alkyl.

According to some embodiments, R⁷ is hydrogen.

According to some embodiments, R⁷ is [(C═O)Oᵣ]ₛaryl.

According to some embodiments, R⁷ is [(C═O)Oᵣ]ₛ(C₁-C₈)-alkyl.

According to some embodiments, R⁶ and R⁷ are each hydrogen.

According to some embodiments, R⁶ and R⁷ are each [(C═O)Oᵣ]ₛaryl.

According to some embodiments, R⁶ and R⁷ are each [(C═O)Oᵣ]ₛ(C₁-C₈)-alkyl.

According to some embodiments, the compound is an enantiomerically pure compound or an enantiomerically enriched compound.

According to some embodiments, the compound of the invention is a compound selected from the group consisting of:
(3-(((((1R,4R)-4-((2-Methoxybenzamido)methyl)-4-(thiophen-3-yl)cyclohexyl)oxy)carbonyl)amino)propyl)triphenylphosphonium iodide,
(3-(((((1S,4S)-4-((2-Methoxybenzamido)methyl)-4-(thiophen-3-yl)cyclohexyl)oxy)carbonyl)amino)propyl)triphenylphosphonium iodide,
(3-(((((1R,4R)-4-((2-Methoxybenzamido)methyl)-4-(thiophen-2-yl)cyclohexyl)oxy)carbonyl)amino)propyl)triphenylphosphonium iodide,
(3-(((((1S,4S)-4-((2-Methoxybenzamido)methyl)-4-(thiophen-2-yl)cyclohexyl)oxy)carbonyl)amino)propyl)triphenylphosphonium iodide,
(3-(((((1R,4R)-4-((2-Methoxybenzamido)methyl)-4-phenylcyclohexyl)oxy)carbonyl)amino)propyl)triphenylphosphonium iodide,
(3-(((((1S,4S)-4-((2-methoxybenzamido)methyl)-4-phenylcyclohexyl)oxy)carbonyl)amino)propyl)triphenylphosphonium iodide,
(4-(((((1R,4R)-4-((2-Methoxybenzamido)methyl)-4-(thiophen-3-yl)cyclohexyl)oxy)carbonyl)amino)butyl)triphenylphosphonium iodide,
(4-(((((1S,4S)-4-((2-Methoxybenzamido)methyl)-4-(thiophen-3-yl)cyclohexyl)oxy)carbonyl)amino)butyl)triphenylphosphonium iodide,
(4-(((((1R,4R)-4-((2-Methoxybenzamido)methyl)-4-(thiophen-2-yl)cyclohexyl)oxy)carbonyl)amino)butyl)triphenylphosphonium iodide,
(4-(((((1S,4S)-4-((2-Methoxybenzamido)methyl)-4-(thiophen-2-yl)cyclohexyl)oxy)carbonyl)amino)butyl)triphenylphosphonium iodide,
(4-(((((1R,4R)-4-((2-Methoxybenzamido)methyl)-4-phenylcyclohexyl)oxy)carbonyl)amino)butyl)triphenylphosphonium iodide, and
(4-(((((1S,4S)-4-((2-Methoxybenzamido)methyl)-4-phenylcyclohexyl)oxy)carbonyl)amino)butyl)triphenylphosphonium iodide.

A compound of the present invention may form stable acid or basic salts, and in such cases administration of a compound as a salt may be appropriate, and pharmaceutically acceptable salts may be made by conventional methods such as those described below.

Examples of salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, dibenzylamine, mopholine, N-ethylmorpholine, N-methylpiperidine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine (i.e., 2-amino-2-hydroxymethyl-propane-1,3-di ol), tris-(2-hydroxyethyl)amine, and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, malonic, salicyclic, ascorbic, benzoic, succinic, suberic, fumaric, mandelic, phthalic, α-glycerophosphoric, benzenesulfonic, p-tolylsulfonic, citric, tartaric, methanesulfonic, and the like. Also included are salts of amino acids such as argininate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the present invention or a salt thereof may exhibit the phenomenon of tautomerism and that the formulae drawings within this specification can represent only one of the possible tautomeric forms. It is to be understood that the invention encompasses any tautomeric form which inhibits K_{V}1.3 channels and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It will be appreciated by those skilled in the art that certain compounds of the present invention contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which posses properties useful in the inhibition of K_{V}1.3 channels, it being well known in the art how to prepare optically-active forms (for example, by resolution of the racemic form by recrystallization techniques, by synthesis from optically-active starting materials, by stereoselective synthesis, by enzymatic resolution, by biotransformation, or by chromatographic separation using a chiral stationary phase).

It is also to be understood that certain compounds of the present invention and salts thereof can exist in solvated as well as unsolvated forms such as, for example, hydrated forms. It is to be understood that the invention encompasses all such solvated forms which inhibit K_{V}1.3 channels.

In addition to salt forms, the invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. A prodrug may improve the physical properties of the parent drug and/or it may also improve overall drug efficacy, for example through the reduction of toxicity and unwanted effects of a drug by controlling its absorption, blood levels, metabolic distribution and cellular uptake.

If a compound of the present invention is represented as a salt, the present invention is intended to include free bases, free acids, or alternative salts of these particular compound.

Moreover, it should be noted that each of these compounds and salts thereof, are also intended to be separate embodiments, and in this regard, each species listed in Examples, and salt thereof, should be considered to be an individual embodiment. Moreover, it should be understood that the present invention is intended to include any novel compound or pharmaceutical composition described herein.

### Nanoparticles

In certain aspects, the compounds of the present invention can be incorporated into nanoparticles. Suitable nanoparticles include a core and one or more of the compounds disclosed herein. The disclosed compounds can be contained or embedded within the core. The disclosed compounds are preferably released from the core at a desired rate. The core is biodegradable and releases the disclosed compounds as the core is degraded or eroded. The targeting moieties preferably extend outwardly from the core so that they are available for interaction with the cellular components, which interactions will target the nanoparticles to the appropriate cells, such as apoptotic cells; organelles, such as mitochondria; or the like.

The core of the nanoparticle can be formed from any suitable component or components. Preferably, the core is formed from hydrophobic components such as hydrophobic polymers or hydrophobic portions or polymers or lipids. In certain examples, the core includes phospholipids which can form micelles having a hydrophobic core and a hydrophilic outer surface. The core can also or alternatively include block copolymers that have hydrophobic portions and hydrophilic portions that can self-assemble in an aqueous environment into particles having the hydrophobic core and a hydrophilic out surface. In certain examples, the core comprises one or more biodegradable polymers or a polymer having a biodegradable portion. Any suitable synthetic or natural biodegradable polymer can be used. Such polymers are recognizable and identifiable by one or ordinary skilled in the art. Non-limiting examples of synthetic, biodegradable polymers include: poly(amides) such as poly(amino acids) and polypeptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable.

Preferably, at least some of the polymers used to form the core are amphiphilic having hydrophobic portions and hydrophilic portions. The hydrophobic portions can form the core, while the hydrophilic regions can for a shell that helps the nanoparticle evade recognition by the immune system and enhances circulation half-life. Examples of amphiphilic polymers include block copolymers having a hydrophobic block and a hydrophilic block. In various examples, the core is formed from hydrophobic portions of a block copolymer, a hydrophobic polymer, or combinations thereof.

Any suitable hydrophilic polymer can form a hydrophilic block of a block copolymer. Examples of suitable hydrophilic polymers include polysaccharides, dextran, chitosan, hyaluronic acid, and the like. In embodiments, polyethylene glycol (PEG) is a hydrophilic polymer used to serve as the hydrophilic portion of a block copolymer. Nanoparticles, as described herein, can be of any suitable size. Generally, the nanoparticles are of a diametric dimension of less than about 999 nanometers, such as less than about 750 nm, less than about 600 nm, less than about 500 nm, less than about 400 nm, less than about 300 nm, or less than about 200 nm. In addition, or alternatively, the nanoparticles can be of a diametric dimension of greater than about 5 nm. In embodiments, the nanoparticles are from about 30 nm to about 300 nm in diameter. In embodiments, the nanoparticles are separated according to size, such as from about 20 nm to about 40 nm, from about 40 nm to about 60 nm, from about 60 nm to about 80 nm, from about 80 nm to about 100 nm, or from about 100 nm to about 150 nm.

Nanoparticles, as described herein, can be synthesized or assembled via any suitable process. Preferably, the nanoparticles are assembled in a single step to minimize process variation. A single step process can include nanoprecipitation and self-assembly. The nanoparticles can be synthesized or assembled by dissolving or suspending hydrophobic components in an organic solvent, preferably a solvent that is miscible in an aqueous solvent used for precipitation. In certain examples, acetonitrile is used as the organic solvent, but any suitable solvent can be used. Hydrophilic components are dissolved in a suitable aqueous solvent, such as water, 4 wt % ethanol, or the like. The organic phase solution can be added drop wise to the aqueous phase solution to nanoprecipitate the hydrophobic components and allow self-assembly of the nanoparticle in the aqueous solvent.

A process for determining appropriate conditions for forming the nanoparticles can be as follows. Briefly, functionalized polymers and phospholipids may be co-dissolved in organic solvent mixtures (in embodiments, the phospholipids or functionalized phospholipids are dissolved in the aqueous solvent). This solution can be added drop wise into hot (e.g., 65° C.) aqueous solvent (e.g., water, 4 wt-% ethanol, etc.), whereupon the solvents will evaporate, producing nanoparticles with a hydrophobic core coated with phospholipids. The phospholipids used at this stage may be a mixture of non-functionalized phospholipids and functionalized phospholipids (e.g., conjugated to targeting moieties) that can also include a hydrophilic polymer component, such as PEG. Once a set of conditions where a high (e.g., >75%) level of compound loading has been achieved, contrast agents or additional therapeutic agents can be included in the nanoprecipitation and self-assembly of the nanoparticles. The size of the nanoparticle produced can be varied by altering the ratio of hydrophobic core components to amphiphilic shell components. The choice of PEGylated lipids and bilayer forming phospholipids can affect resulting nanoparticle size. PEGylated lipids are known to form small micellar structures because of surface tension imposed by the PEG chains. NP size can also be controlled by changing the polymer length, by changing the mixing time, and by adjusting the ratio of organic to the phase. Prior experience with NPs from PLGA-b-PEG of different lengths suggests that NP size will increase from a minimum of about 20 nm for short polymers (e.g., PLGA3000-PEG750) to a maximum of about 150 nm for long polymers (e.g., PLGA1000,000-PEG 10,000). Thus, molecular weight of the polymer will serve to adjust the size.

NP surface charge can be controlled by mixing polymers with appropriately charged end groups. Additionally, the composition and surface chemistry can be controlled by mixing polymers with different hydrophilic polymer lengths, branched hydrophilic polymers, or by adding hydrophobic polymers. Once formed, the nanoparticles can be collected and washed via centrifugation, centrifugal ultrafiltration, or the like. If aggregation occurs, NPs can be purified by dialysis, can be purified by longer centrifugation at slower speeds, can be purified with the use surfactant, or the like.

Once collected, any remaining solvent can be removed and the particles can be dried, which should aid in minimizing any premature breakdown or release of components. The NPs can be freeze dried with the use of bulking agents such as mannitol, or otherwise prepared for storage prior to use.

### Pharmaceutical Compositions

The compounds of the present invention can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Compositions containing the compound described herein or derivatives thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants. These compositions can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hardfilled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients. The disclosed compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

Liquid dosage forms for oral administration of the compounds described herein or derivatives thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents. Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component. Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as can be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These can include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al, J. Pharm. Sci. (1977) 66, 1, which is incorporated herein by reference in its entirety, at least, for compositions taught herein.)

Administration of the compounds and compositions described herein or pharmaceutically acceptable salts thereof to a subject can be carried out using therapeutically effective amounts of the compounds and compositions described herein or pharmaceutically acceptable salts thereof as described herein for periods of time effective to treat a disorder.

The effective amount of the compounds and compositions described herein or pharmaceutically acceptable salts thereof as described herein can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day.

Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. The expression effective amount, when used to describe an amount of compound in a method, refers to the amount of a compound that achieves the desired pharmacological effect or other effect, for example an amount that results in enzyme inhibition.

Those of skill in the art will understand that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

In a preferred embodiment, the pharmaceutical composition is in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The pharmaceutical compositions may comprising a further anticancer agent, such as a anticancer agent is selected from the group consisting of 13-cis-Retinoic Acid, 2-Amino-6-Mercaptopurine, 2-CdA, 2-Chlorodeoxy adenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ. Alkeran, All-transretinoic acid, Alpha interferon, Altretamine, Amethopterin, Amifostine, Aminoglute thimide, Anagrelide, Anandron, AnastroZolc, Arabininosyl cytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabinc liposomal, Cytosar-U. Cytoxan, Dacarbazine, Dactinomycin, Darbepoctin alfa, Daunomycin, Daunorubicin, Daunorubicin hydrochloride, Daunorubicin liposomal, DaunoXome, Decadron, Delta Cortef, Deltasone, Denileukin diftitox, DepoCyt, Dexamethasonc, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, ErbituX, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate. Oncospar. Oncovin, Ontak, Onxal, Oprevelkin, Orapred. Orasone, Oxaliplatin, Paclitaxel, Pamidronate, Panretin, Paraplatin, Peodiapred. PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustineimplant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solumedrol, STI-571, Streptozocin, Tamoxifen, Targretin, Taxol. Taxotere, Temodar, Temozolomide, Teniposide, TESPA, Thalidomide, Thalomid, TheraCys. Thioguanine. Thioguanine Tabloid. Thiophosphoamide. Thioplex. Thiotepa, TICE. Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium Succinate, Hydrocortone phosphate, Hydroxyurea, Ibritumomab, Ibritumomab Tiuxetan, Idamycin, Idarubicin, Ifex, IFN-alpha, Ifosfamide, IL2, IL-11, Imatinib mesylate. Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C. Mitoxantrone, M-Prednisol, MTC, MTX, Mustargcn, Mustine, Mutamycin, Myleran, Iressa, Irinotecan, Isotretinoin, Kidrolasc, Lanacort, L-asparaginase, and LCR.

The afore-mentioned formulations will be prepared using standard methods such as those described or referred to in the US Pharmacopoeia and similar reference texts.

### Medical Uses

As stated above, a compound of the present invention is particularly useful as a medicament, e.g. as a medicament for the treatment or prevention of a disease or condition that is ameliorated by the inhibition of mitochondrial K_{V}1.3 ion channels.

The present invention thus provides a compound of the present invention for use in medicine.

More specifically, the present invention provides a compound of the present invention, including but not limited to those specified in the examples, for use in the treatment or prevention of cancer.

Non-limiting examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, smooth muscle cancer, skeletal muscle cancer, prostate cancer, renal cancer, skin cancer, testicular cancer, cancer and/or tumors of the anus, bile duct cancer, bone cancer, bone marrow cancer, eye cancer, gall bladder cancer, kidney cancer, mouth cancer, laryngeal cancer, esophagus cancer, stomach cancer, cervix cancer, mesothelioma cancer, neuroendocrine cancer, spinal cord, thyroid cancer, vaginal cancer, vulva cancer, uterus cancer, liver cancer, muscle cancer, blood cell cancer (including lymphomas and leukemias).

Specific cancers contemplated for treatment include carcinomas, Kaposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Preferred cancers treatable by the compounds and compositions described herein are lung, breast, brain, ovarian, lymphoma, leukemia, smooth muscle, skeletal muscle, head and neck, pancreatic, and cervical, colon and rectum, endometrial, esophagus, liver, penile, skin melanoma, skin-nonmelanoma, stomach, testicular, vaginal, uterine, vulvar, paranasal cancer, oropharyngeal and laryngeal cancers.

Particularly preferred cancers include breast, colon, and prostate tumors, melanoma, smooth muscle cancer, skeletal muscle cancer, chronic lymphocytic leukemia, glioblastoma, and pancreatic ductal adenocarcinoma.

Provided herein are methods of treating, preventing, or ameliorating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer.

The methods of treatment or prevention described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein.

The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

Another aspect of the present invention pertains to a pharmaceutical composition which comprises a compound of the present invention, or a pharmaceutically acceptable salt or hydrate thereof, and a pharmaceutically acceptable excipient and/or carrier. The pharmaceutical composition may be used in the treatment or prevention of any one of the diseases mentioned above.

As a general remark, the use of “comprising” and “comprises” as used herein, especially when defining the contents of a medicament or a pharmaceutical formulation is to be understood as also disclosing “consisting of” and “consists of” respectively etc. Thus, this also includes that the contents of the respective medicament or pharmaceutical formulation are then to be also understood to be limited to the exact contents preceded by this “comprising” or “comprises” etc.

### Process

In a further aspect the present invention provides a process of preparing a compound of the present invention.

If not commercially available, the necessary starting materials for the procedures such as those described below may be made by procedures which are selected from standard organic chemistry techniques, techniques which are analogous to the synthesis of known structurally similar compounds, or techniques, which are analogous to the procedures described in the examples.

It will also be appreciated that in some of the reactions mentioned herein it may be necessary/desirable to protect any sensitive groups in compounds. The instances where protection is necessary or desirable are known to those skilled in the art, as are suitable methods for such protection.

Example of a suitable protecting group for a hydroxy group is, for example, an acyl group, for example an alkanol group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanol or an aroyl group may be removed, for example, by hydrolysis with a suitable base such as an alkali metal hydroxide, for example lithium or sodium hydroxide. Alternatively, a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively, an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris (trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

The protecting groups may be removed at any convenient stage in the synthesis using conventional techniques well known in the chemical art, or they may be removed during a later reaction step or work-up.

Methods for preparing the compounds of this invention are illustrated in the following schemes. Other synthetic protocols will be readily apparent to those skilled in the art.

Methods for preparing the compounds of this invention are illustrated in the following schemes. Other synthetic protocols will be readily apparent to those skilled in the art.

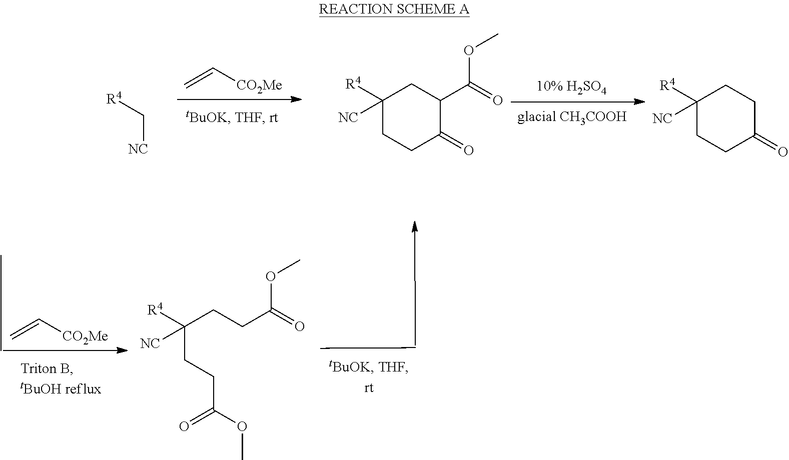

The substituted or unsubstituted aryl- or heteroaryl-acetonitrile substrates shown in Scheme A, which are starting materials to obtain the compounds of this invention, are commercially available or can be prepared by procedures well known in art. As shown in Scheme A, the aryl- or heteroaryl-acetonitrile precursors are converted to 4,4-disubstituted-2-carbomethoxycyclohexanones intermediates via efficient two-pot or one-pot methods. The most commonly used procedure in the literature is two-step procedure, initially reported by Irie, H., Tsuda Y.; Uyeo, S. J.; Chem. Soc. 1446 (1959). In this reaction sequence, aryl- or heteroaryl-acetonitriles are boiled at reflux to undergo double Michael addition in the presence of methyl acrylate and benzyl-(trimethyl)ammoniumhydroxide (Triton B) to afford diester intermediates. Subsequently, intermediates are deprotonated with bases such as 95% sodium hydride or potassium tert-butoxide in a separate step to gain 4-heteroaryl-4-cyano-2-carbomethoxycyclohexanone derivatives via Dieckmann condensation. Alternatively, 4-aryl- or 4-heteroaryl-4-cyano-2-carbomethoxycyclohexanones can be prepared via one-pot synthesis in the presence of methyl acrylate and potassium tert-butoxide in THF at room temperature as described in DeGraffenreid, M. R. et al.; J. Org. Chem. 72, 19, 7455-7458, 2007. 2-carbomethoxy group can be removed from intermediates to gain the corresponding 4-aryl- or 4-heteroaryl-4-cyano cyclohexanone derivatives by stirring at 100° C. in 10% sulfuric acid and glacial acetic acid.

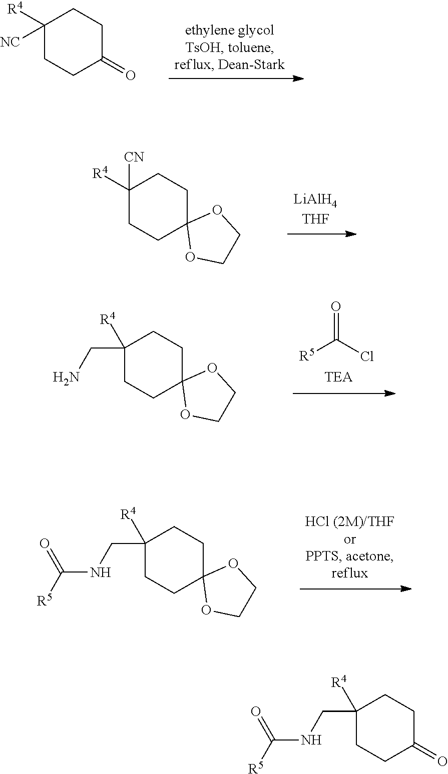

As shown in Scheme B, the protected 4-cyano-4-heteroaryl/aryl cyclohexanone precursors, are prepared according to procedures described and cited by Swenton, J. S.; Blankenship, R. M.; and Sanitra, R; J. Am. Chem. Soc., 97, 17, 4941-4947, 1975 with ethane-1,2-diol and p-toluenesulfonic acid (TsOH). Nitrile group can be reduced with LiAlH₄ in an aprotic solvent such as tetrahydrofuran (THF) to the corresponding primary amines. The amine derivatives can be acylated with acid chlorides in aprotic solvents such as dichloromethane with a base such as triethylamine to give the corresponding benzamides. The acid chlorides can be prepared from carboxylic acids in reagents such as oxalyl chloride or thionyl chloride. Alternatively, amides can be prepared by reaction of benzoic acids with the amine using standard coupling conditions as described in March, J.; Advanced Organic Chemistry, 4th ed., John Wiley & Sons, New York, pp. 417-424 (1992). The ketal group is removed by stirring in acetone with pyridinium p-toluenesulfonate (PPTS). Alternatively, ketal group can be removed under dilute acidic conditions such as 2 M solution of HCl, which are described in March, J.; Advanced Organic Chemistry, 4th ed., John Wiley & Sons, New York, pp. 372-375 (1992).

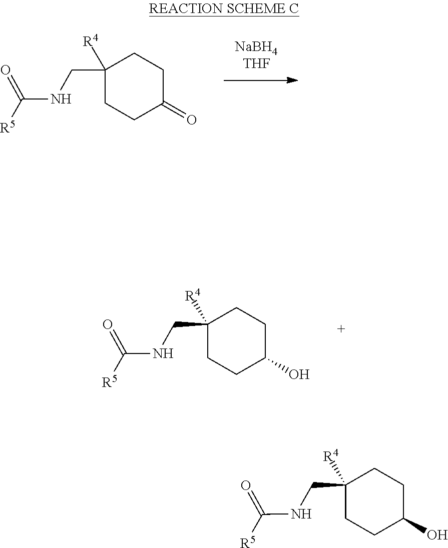

As presented in Scheme C, the ketone group is selectively reduced with NaBH₄ in solvents such as THF as described in March, J.; Advanced Organic Chemistry, 4th ed., John Wiley & Sons, New York, pp. 1206-1208 (1992) to afford a diasteroisomeric mixture of alcohols that can be separated by standard chromatography methods.

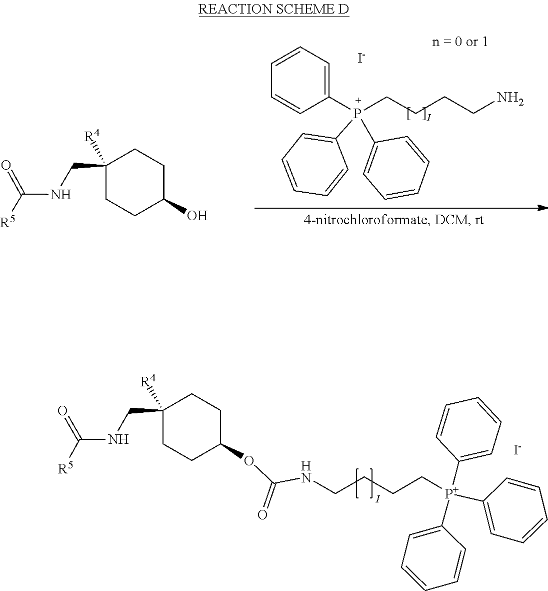

As presented in Scheme D, carbamate or carbonate derivatives are prepared by first reacting the alcohol analogues with 4-nitrochloroformates to provide 4-nitrophenylcarbonate intermediate which can be reacted with amines to yield carbamates or with alcohols to give corresponding carbonate derivatives. In alternate approaches, carbamate derivatives can also be prepared by commercially available carbamoyl chlorides, isocyanates or by first reacting the C4 alcohol derivatives with carbonyldiimidazole to obtain imidazolecarbonyl intermediate which is then reacted with an alcohol (R4′OH) or amine (R4′R4″NH) to give the corresponding carbamate or carbonate derivatives.

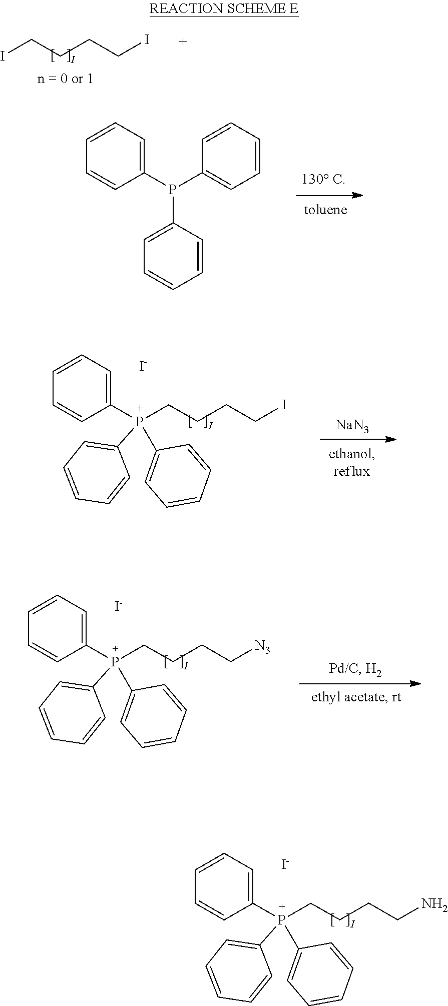

The diiodo substrates shown in Scheme E, which are the starting materials to obtain the compounds of this invention, are commercially available or can be prepared by procedures well known in art. As shown in Scheme E, the diiodo precursors are boiled under reflux with triphenylphosphine in solvents such as toluene to gain the corresponding monoiodotriphenylphosphine⁺ iodide salts. These intermediates are further converted to the corresponding azide derivatives via nucleophilic substitution in solvents such as ethanol at reflux. Finally, azide intermediate is reduced to the corresponding amine derivative by hydrogenation with palladium catalyst. This and other procedures are described in March, et al., Advanced Organic Chemistry, 4th ed., John Wiley & Sons, New York, pp. 428, 1219, 1992.

The present invention thus provides a process for preparing a compound of the present invention process comprising (wherein the variables are as defined above unless otherwise stated):
Process step a) transformation of a compound of formula (XI)

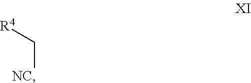

wherein R⁴ is as defined above,
to a compound of formula (XII)

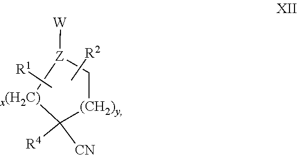

wherein R¹, R², R⁴, x and y are as defined above, and Z is selected from CH or N, and W is selected from —H, hydroxyl, —NHR^{B}, —COOH, —COO(C1-C6), —CHR⁸, —SO₃H or —SH,
and
Process step b) transformation of a compound of formula (XII)

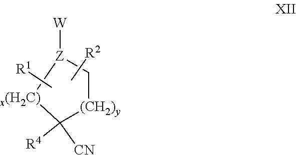

to a compound of formula (XIII)

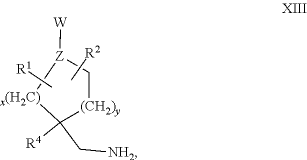

wherein R¹, R², R⁴, Z, W, x and y are as defined above,
and
Process step c) transformation of a compound of formula (XIII)

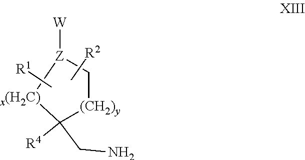

to a compound of formula (XIV)

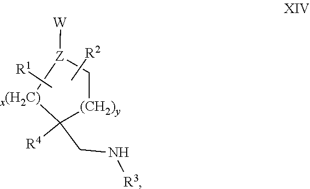

wherein R¹, R², R³, R⁴, Z, W, x and y are as defined above,
and
Process step d) reacting a compound of formula (XIV) with a compound of formula (XV):

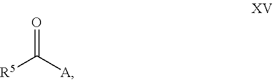

wherein A is selected from hydroxyl, alkoxy, halogen (preferably Cl, Br or I),
to a compound of formula (XVI):

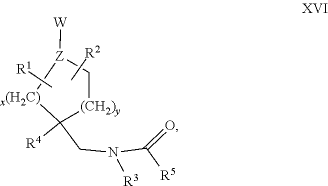

wherein R¹, R², R³, R⁴, R⁵, Z, W, x and y are as defined above,
and
Process step e) reacting a compound of formula (XVI) with a compound of formula (XVII):

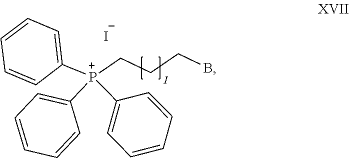

wherein B is selected from hydroxyl, mesyl, tosyl, halogen (preferably I),
to a compound of formula (XVIII):

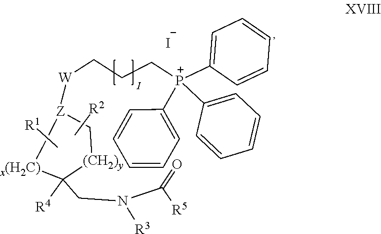

wherein R¹, R², R³, R⁴, R⁵, W, Z, x and y are as defined above.

### EXAMPLES

The invention is illustrated but not limited by the following Examples in which unless otherwise stated:
(i) evaporation was carried out by rotary evaporation in vacuo and work-up procedures were carried out after removal of residual solids after filtration;
(ii) operations were generally carried out at ambient temperature, that is typically between 18 and 26° C. and without exclusion of air unless otherwise stated, or unless skilled person would otherwise work under an inert atmosphere;
(iii) flash column chromatography was used to purify compounds and was performed on Merck Silica Gel 60 unless otherwise stated;
(iv) yields are given for illustration only and are not necessarily the maximum attainable;
(v) the structure of the end-products was generally confirmed by NMR and mass spectral techniques; proton NMR spectra is quoted and was determined using a Bruker Avance III 400 MHz spectrometer operating at field strength of 400 MHz. Chemical shifts are reported in part per million downfield from tetramethylsilane as an internal standard (δ scale) and peak multiplicities are shown thus: s, singlet; d, doublet; dd, doublet of doublets; dt, doublet of triplets; t, triplet; m, multiplet; br, broad;
(vi) mass spectra were obtained using an Exactive Plus Orbitrap mass spectrometer (Thermo Fisher Scientific, Waltham, Massachusetts, ZDA).
(vii) each intermediate was generally purified to the standard required for the subsequent stage and was characterised in sufficient detail to confirm that the assigned structure was correct; purity was assessed by high pressure liquid chromatography, thin layer chromatography, or NMR and identity was determined by mass spectrometry and NMR spectroscopy as appropriate.

### General Synthetic Chemistry Experimental Protocols

### General Procedure A: Synthesis of Diester Intermediates

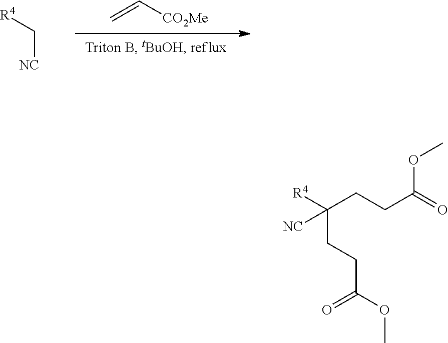

Corresponding aromatic or heteroaromatic acetonitrile (75 mmol, 1.0 equiv) and methyl acrylate (375 mmol, 5.0 equiv) were dissolved in tert-butanol (45 mL) at room temperature and heated to boiling point. The heating source was then removed and benzyltrimethylammonium hydroxide (75 mmol, 1.0 equiv), dissolved in tert-butanol (10 mL), was added dropwise at room temperature. The reaction mixture was stirred boiled under reflux for 4 h and then cooled to room temperature overnight. Next day toluene (100 mL) and water (70 mL) were added to reaction mixture. The organic phase was separated and washed with water (2×70 mL), saturated brine solution (50 mL), dried over Na₂SO₄, filtered, and the solvent evaporated under reduced pressure. The product was used without further purification.

### General Procedure B: Synthesis of 4-aryl-4-cyano-2-carbomethoxycyclohexanone Derivatives

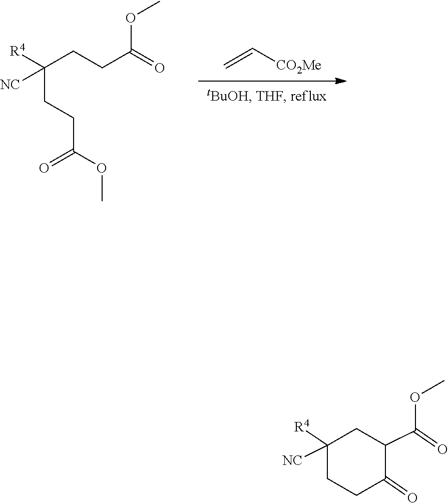

Appropriate cyanothiopheneheptanedioate (61 mmol, 1.0 equiv) was dissolved in anhydrous THF (250 mL) under argon atmosphere. Potassium tert-butoxide (122 mmol, 2 equiv) was added in portions with ice cooling. The reaction mixture was stirred under reflux for 5 hours and cooled to room temperature overnight. Next day 2.5 M acetic acid (220 mL) was added dropwise with ice cooling. The batch was mixed with toluene (150 mL). Organic phase was separated and washed with saturated aqueous NaHCO₃ solution (3×100 mL), water (3×100 mL) and saturated brine solution (75 mL). After drying over Na₂SO₄, precipitate was filtered off and the solvent was evaporated under reduced pressure. The product was used without further purification unless stated otherwise.

### General Procedure C: Synthesis of 4,4-Disubstituted Cyclohexanones

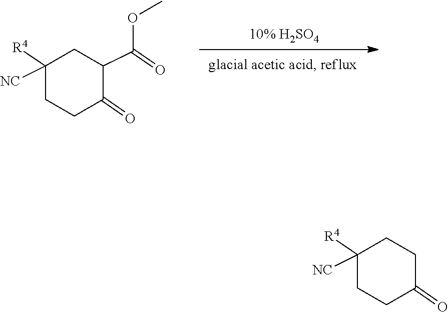

Corresponding methyl 2-oxocyclohexane-1-carboxylate (47 mmol, 1.0 equiv) was dissolved in 10% sulfuric acid (170 mL) and glacial acetic acid (380 mL). The reaction mixture was boiled at 100° C. for 24 hours. The batch was then cooled to room temperature and diluted with water (500 mL) on ice bath. The water phase was extracted with ethyl acetate (3×150 mL) and combined organic phases were thoroughly washed with saturated aqueous NaHCO₃ solution (5×100 mL), water (5×100 mL), saturated brine solution (100 mL), dried over Na₂SO₄, and evaporated. When ethyl acetate (25 mL) was added to crude product, white precipitate was formed. White precipitate was removed by filtration and dried. The product was additionally purified by flash column chromatography.

### General Procedure D: Introduction of Protection Group to Ketone Derivatives

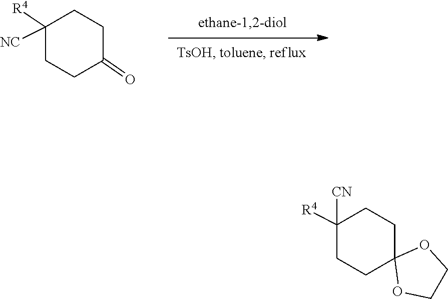

Ketone derivative (29 mmol, 1.0 equiv) was dissolved in toluene (300 mL). Ethane-1,2-diol (290 mmol, 10.0 equiv) and p-toluenesulfonic acid (0.58 mmol, 0.02 equiv.) were added to reaction mixture. The flask was boiled at 140° C. in Dean-Stark apparatus overnight. Next day the flask was cooled to room temperature and the solvent was evaporated. Product was dissolved in ethyl acetate (400 mL) and washed with saturated aqueous NaHCO₃ solution (2×150 mL), water (2×150 mL) and saturated brine solution (150 mL). Organic phase was dried with Na₂SO₄, filtered and evaporated under reduced pressure. The product was additionally purified by flash column chromatography.

### General Procedure E: Reduction of Carbonitrile to Amine Derivatives

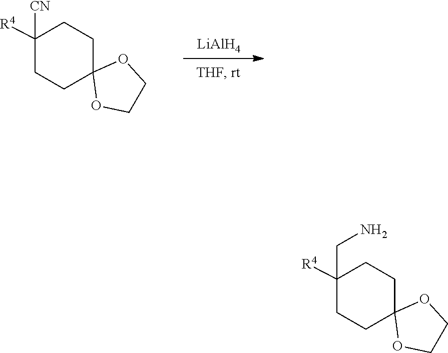

Carbonitrile intermediate (27 mmol, 1.0 equiv.) was dissolved in anhydrous THF (100 mL) under argon atmosphere with ice cooling. LiAlH₄ (54 mmol, 2.0 equiv.) was added in portions on ice bath and batch was stirred at room temperature overnight. For workup diethylether (300 mL) was added to flask with ice cooling and then saturated brine solution (5-10 mL) was slowly added while the batch was stirred on ice bath. Residual water was removed by addition of Na₂SO₄. Precipitate was filtered off and additionally washed with diethylether. Organic solvent was removed under reduced pressure and the product was used without further purification unless stated otherwise.

### General Procedure F: Synthesis of Benzamide Analogues

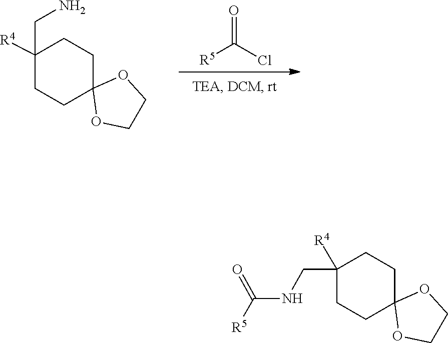

Benzoic acid derivate (26 mmol, 1.0 equiv) was dissolved in dichloromethane (100 mL) with ice cooling. Oxalyl chloride (78 mmol, 3.0 equiv) was added dropwise, followed by 5 drops of DMF. The batch was stirred at room temperature overnight and next day the solvent was evaporated. Appropriate amine (26 mmol, 1.0 equiv) and Et₃N (78 mmol, 3.0 equiv.) were dissolved in dichloromethane (75 mL) with ice cooling, followed by addition of benzoyl chloride intermediate (26 mmol, 1.0 equiv), dissolved in dichloromethane (75 mL). Reaction mixture was stirred at room temperature overnight. Organic phase was then diluted with 75 mL of dichloromethane and washed with saturated aqueous NaHCO₃ solution (2×50 mL), 1M aqueous HCl solution, water (2×50 mL), saturated brine solution (50 mL), dried over Na₂SO₄, and organic phase was then removed under reduced pressure. The product was used without further purification unless stated otherwise.

### General Procedure G: Removal of Protection Group from Ketone

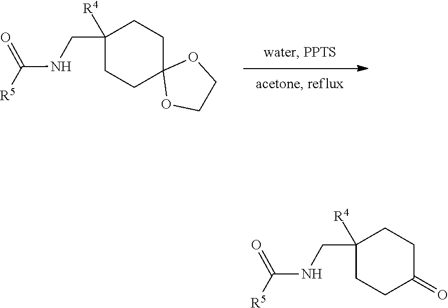

Benzamide analogue (23 mmol, 1.0 equiv) was dissolved in acetone (150 mL), followed by the addition of pyridinium p-toluenesulfonate (PPTS) (2.3 mmol, 0.1 equiv) and water (20 mL). The reaction mixture was stirred at reflux for 48 h, and then the solvent was evaporated. The residue was dissolved in dichloromethane (200 mL) and washed with aqueous NaHCO₃ solution (1×50 mL), 1 M aqueous HCl solution (1×50 mL), water (2×50 mL), and saturated brine solution (50 mL). Organic phase was dried over Na₂SO₄, filtered and the solvent removed under reduced pressure. The product was purified by flash column chromatography.

### General Procedure H: Reduction of Ketone Group to Hydroxyl Group

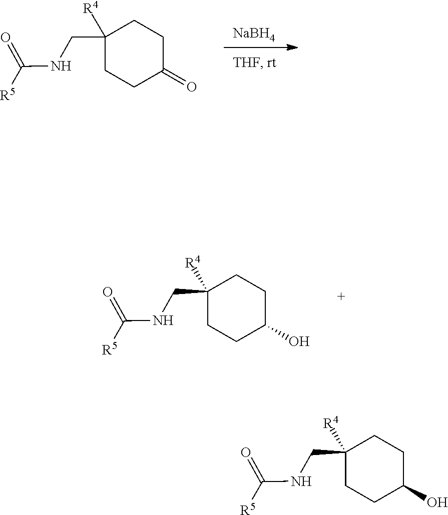

Benzamide derivative (17 mmol, 1.0 equiv) was dissolved in anhydrous THF (100 mL) under argon atmosphere with ice cooling. NaBH₄ (34 mmol, 2.0 equiv) was then added in portions with ice cooling and the batch was stirred at room temperature overnight. Next day 1M aqueous HCl solution (100 mL) was added to reaction mixture with ice cooling and extracted with dichloromethane (2×100 mL). Combined organic phases were then washed with water (50 mL), dried over Na₂SO₄ and removed under reduced pressure. Product was purified by flash column chromatography. Trans and cis derivatives were separated by flash column chromatography.

### General Procedure I: Synthesis of Carbamate Derivatives from Alcohols

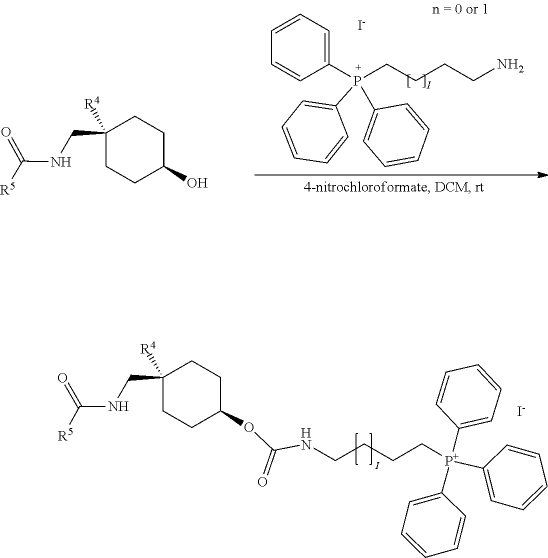

Hydroxyl analogue (0.6 mmol or 1.2 mmol, 1.0 equiv.) was dissolved in dichloromethane (50 mL) and then Et₃N (3 mmol or 6 mmol, 5.0 equiv) was slowly added. The flask was stirred at room temperature for 5 minutes and then 4-nitrophenyl chloroformate (1.2 mmol or 2.4 mmol, 2.0 equiv.) was added in portions. Reaction mixture was stirred at room temperature overnight and then washed with water (25 mL), 1M aqueous HCl solution (25 mL), and saturated brine solution (25 mL). Organic phase was dried over Na₂SO₄ and removed under reduced pressure. Intermediate (0.3 mmol or 0.6 mmol, 1.0 equiv.) was dissolved in dichloromethane (50 mL) and then amine (3 mmol or 6 mmol, 10.0 equiv.) was added at room temperature. The flask was stirred at room temperature overnight and next day washed with water (25 mL), 1 M aqueous HCl solution (25 mL), and saturated brine solution (25 mL). Organic phase was dried over Na₂SO₄, filtered and the solvent removed under reduced pressure. Product was additionally purified by flash column chromatography.

### General Procedure J: Synthesis of Monoidoalkyltriphenylphosphine⁺ iodide salts

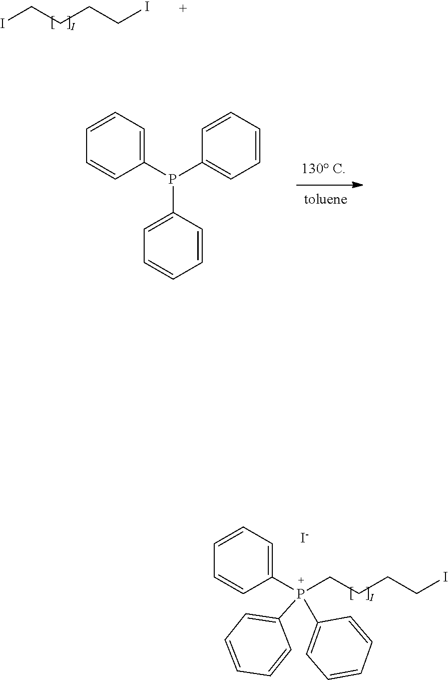

Triphenylphosphine (TPP) (11.5 mmol, 1.0 equiv) and corresponding diiodo derivative (23 mmol, 2.0 equiv) were dissolved in toluene (45 mL) at room temperature and heated to 130° C. The reaction mixture was stirred boiled under reflux for 48 h and then cooled to room temperature. Solvent was removed under reduced pressure and small amount of toluene (10 mL) was added to reaction mixture to obtain a precipitate, which was filtered off and the product was used without further purification.

### General Procedure K: Synthesis of Azide Derivatives

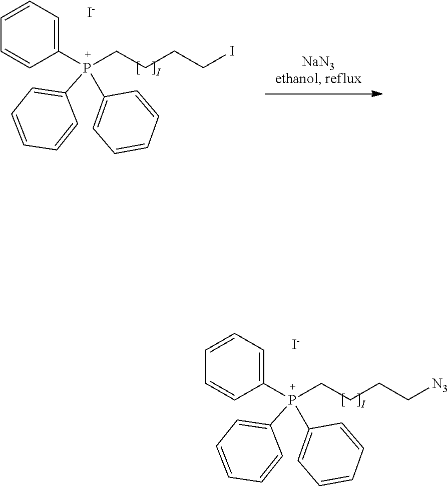

Corresponding monoiodoalkyltriphenylphosphine⁺ iodide analogue (10.8 mmol, 1.0 equiv) was dissolved in ethanol (25 mL) and after 5 minutes sodium azide (21.6 mmol, 2.0 equiv) was added to reaction mixture. The flask was boiled under reflux for 24 hours and then cooled to room temperature. Ethanol (20 mL) was added to reaction mixture and organic phase was washed with water (10 mL) to remove sodium azide. Solvent was then removed under reduced pressure and product was used without further purification.

### General Procedure L: Reduction of Azide to Amine Derivatives

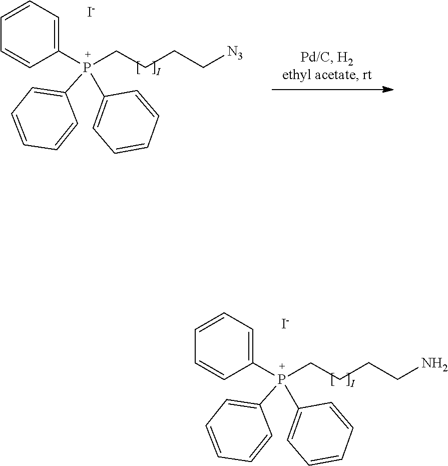

Azide intermediate (1.1 mmol, 1.0 equiv.) was dissolved in ethyl acetate (50 mL) and purged under a stream of argon for 10 min. Catalytic amount of Pd/C (10% load on carbon, 10-20% [w/w] calculated to the starting material) was added and the resulting suspension mixture was stirred under H₂(g) atmosphere at room temperature for 16-24 h. The catalyst was removed by filtration through Celite and evaporated to obtain crude product. The amine intermediate (1 mmol) was used without further purification unless stated otherwise.

### Example 1

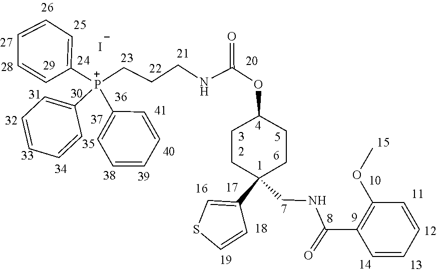 [SMILES: COc1ccccc1C(=O)NC[C@]1(c2ccsc2)CC[C@@H](OC(=O)NCCC[P+](c2ccccc2)(c2ccccc2)c2ccccc2)CC1 ; [I-]]

### Step 1. Dimethyl 4-cyano-4-(thiophen-3-yl)heptanedioate

Synthesized from 2-(thiophen-3-yl)acetonitrile (9.98 mL, 75.0 mmol, 1.0 equiv), methyl acrylate (34.00 mL, 375.2 mmol, 5.0 equiv) and benzyltrimethylammonium hydroxide (13.20 mL, 75.0 mmol, 1.0 equiv.) via general procedure A. Product was used without further purification. Yield: 81% (18.00 g); pale yellow oil. ¹H NMR (400 MHz, CDCl₃): δ 2.15-2.37 (m, 6H), 2.41-2.61 (m, 2H), 3.63 (s, 6H), 6.97 (dd, J₂=5.1 Hz, J₂=1.5 Hz, 1H), 7.33 (dd, J₂=3.0 Hz, J₂=1.5 Hz, 1H), 7.40 (dd, J₂=5.1 Hz, J₂=3.0 Hz, 1H).

### Step 2. Methyl 5-cyano-2-oxo-5-(thiophen-3-yl)cyclohexane-1-carboxylate

Synthesized from dimethyl 4-cyano-4-(thiophen-3-yl)heptanedioate (18.00 g, 61.0 mmol, 1.0 equiv) and potassium tert-butoxide (13.69 g, 122.0 mmol, 2 equiv) via general procedure B. The product was used without further purification. Yield: 77% (12.40 g); pale yellow solid. ¹H NMR (400 MHz, DMSO): δ 2.17-2.27 (m, 1H), 2.29-2.37 (m, 1H), 2.42-2.48 (m, 1H), 2.55-2.65 (m, 2H), 2.71 (dd, J₂=15.8 Hz, J₂=1.0 Hz, 1H), 2.94 (d, J=15.8 Hz, 1H), 3.75 (s, 3H), 7.31 (dd, J₂=5.0 Hz, J₂=1.5 Hz, 1H), 7.63 (dd, J₂=2.9 Hz, J₂=1.5 Hz, 1H), 7.65 (dd, J₂=5.0 Hz, J₂=3.0 Hz, 1H). HRMS (ESI+): m/z calcd for [M+H]⁺ 264.0689; found 264.0682.

### Step 3. 4-Oxo-1-(thiophen-3-yl)cyclohexane-1-carbonitrile

Synthesized from methyl 5-cyano-2-oxo-5-(thiophen-3-yl)cyclohexane-1-carboxylate (12.40 g, 47.0 mmol, 1.0 equiv), 10% sulfuric acid (170 mL) and glacial acetic acid (380 mL) via general procedure C. Column chromatography, EtOAc/n-hex=1/3 (v/v). Yield: 62% (6.00 g); pale yellow solid. ¹H NMR (400 MHz, CDCl₃): δ 2.20-2.31 (m, 2H), 2.51-2.61 (m, 4H), 2.80-2.93 (m, 2H), 7.16 (dd, J₂=5.1 Hz, J₂=1.5 Hz, 1H), 7.36 (dd, J₂=3.0 Hz, J₂=1.5 Hz, 1H), 7.42 (dd, J₂=5.1 Hz, J₂=3.0 Hz, 1H). HRMS (ESI+): m/z calcd for [M+H]⁺ 206.0634; found 260.0628.

### Step 4. 8-(Thiophen-3-yl)-1,4-dioxaspiro[4.5]decane-8-carbonitrile

Synthesized from 4-oxo-1-(thiophen-3-yl)cyclohexane-1-carbonitrile (5.95 g, 29.0 mmol, 1.0 equiv), ethane-1,2-diol (16.2 mL, 290.0 mmol, 10.0 equiv) and p-toluenesulfonic acid (86.10 mg, 0.5 mmol, 0.02 equiv.) via general procedure D. Column chromatography, EtOAc/n-hex=1/3 (v/v). Yield: 96% (6.70 g); white solid. ¹H NMR (400 MHz, CDCl₃): δ 1.80-1.89 (m, 2H), 2.01-2.18 (m, 4H), 2.18-2.26 (m, 2H), 3.93-3.98 (m, 2H), 3.98-4.03 (m, 2H), 7.15 (dd, J₂=5.1 Hz, J₂=1.5 Hz, 1H), 7.30 (dd, J₂=3.0 Hz, J₂=1.5 Hz, 1H), 7.35 (dd, J₂=5.1 Hz, J₂=3.0 Hz, 1H). HRMS (ESI+): m/z calcd for [M+H]⁺ 250.0896; found 250.0890.

### Step 5. (8-(Thiophen-3-yl)-1,4-dioxaspiro[4.5]decan-8-yl)methanamine

Synthesized from 8-(thiophen-3-yl)-1,4-dioxaspiro[4.5]decane-8-carbonitrile (6.70 g, 27.0 mmol, 1.0 equiv) and LiAlH₄ (2.05 g, 54.0 mmol, 2.0 equiv) via general procedure E. The product was used without further purification. Yield: 96% (6.60 g); pale yellow oil. ¹H NMR (400 MHz, CDCl₃): δ 0.96 (brs, 2H), 1.55-1.70 (m, 4H), 1.70-1.79 (m, 2H), 2.09-2.16 (m, 2H), 2.68 (s, 2H), 3.88-3.97 (m, 4H), 7.01 (dd, J₂=5.0 Hz, J₂=1.4 Hz, 1H), 7.03 (dd, J₂=3.0 Hz, J₂=1.4 Hz, 1H), 7.31 (dd, J₂=5.0 Hz, J₂=3.0 Hz, 1H). HRMS (ESI+): m/z calcd for [M+H]⁺ 254.1209; found 254.1201.

### Step 6. 2-Methoxy-N-((8-(thiophen-3-yl)-1,4-dioxaspiro[4.5]decan-8-yl)methyl)benzamide

Synthesized from (8-(thiophen-3-yl)-1,4-dioxaspiro[4.5]decan-8-yl)methanamine (6.60 g, 26.0 mmol, 1.0 equiv), 2-methoxybenzoyl chloride (4.40 g, 26.0 mmol, 1 equiv) and Et₃N (10.9 mL, 78.0 mmol, 3.0 equiv.) via general procedure F. Column chromatography, EtOAc/n-hex=1/1 (v/v). Yield: 89% (8.90 g); white solid. ¹H NMR (400 MHz, CDCl₃): δ 2.07-2.18 (m, 2H), 2.30-2.44 (m, 4H), 2.45-2.55 (m, 2H), 3.76 (s, 3H), 3.77 (d, J=6.3 Hz, 2H), 3.85-3.96 (m, 4H), 6.92 (d, J=8.4 Hz, 1H), 7.07 (td, J=7.9, 1.0 Hz, 1H), 7.18 (dd, J₁=5.0 Hz, J₂=1.4 Hz, 1H), 7.21 (dd, J₁=2.9 Hz, J₂=1.4 Hz, 1H), 7.40-7.48 (m, 2H), 7.75 (brs, 1H), 8.20 (dd, J₁=7.8 Hz, J₂=1.8 Hz, 1H). HRMS (ESI+): m/z calcd for [M+H]⁺ 388.1577; found 388.1565. HRMS (ESI+): m/z calcd for [M+H]⁺388.1577; found 388.1565.

### Step 7. 2-Methoxy-N-((4-oxo-1-(thiophen-3-yl)cyclohexyl)methyl)benzamide

Synthesized from 2-methoxy-N-((8-(thiophen-3-yl)-1,4-dioxaspiro[4.5]decan-8-yl)methyl)benzamide (8.90 g, 23.0 mmol, 1.0 equiv), pyridinium p-toluenesulfonate (0.58 g, 2.3 mmol, 0.1 equiv) and water (20 mL) via general procedure G. Column chromatography, EtOAc/n-hex=1/1 (v/v). Yield: 75% (5.90 g); white solid; ¹H NMR (400 MHz, CDCl₃): δ 2.08-2.18 (2H, m, Hₐ-2,6), 2.30-2.44 (4H, m, Hₐ-3,5, Hₑ-2,6), 2.45-2.54 (2H, m, Hₑ-3,5), 3.75 (3H, s, OCH₃), 3.76 (2H, d, J=7.0 Hz, H-7′, H-7″), 6.92 (1H, d, J=8.3 Hz, H-11), 7.06 (1H, t, J=7.6 Hz, H-13), 7.18 (1H, dd, J₂=5.0 Hz, J₂=1.3 Hz, H-18), 7.21 (1H, dd, J₂=2.9 Hz, J₂=1.4 Hz, H-16), 7.39-7.48 (2H, m, H-12, H-19), 7.75 (1H, t, J=5.3 Hz, NHCO), 8.20 (1H, dd, J₂=7.8 Hz, J₂=1.8 Hz, H-14). ¹³C NMR (100 MHz, CDCl₃): δ 34.15 (C-2,6), 37.80 (C-3,5), 41.18 (C-1), 49.28 (C-7), 55.70 (C-15), 111.24 (C-11), 121.01 (C-9), 121.39 (C-13), 121.61 (C-16), 126.24 (C-18), 126.79 (C-19), 132.53 (C-14), 133.00 (C-12), 144.68 (C-17), 157.51 (C-10), 165.43 (C-8), 211.14 (C-4). HRMS (ESI+): m/z calcd for [M+H]⁺ 344.1315; found 344.1307. HPLC purity, 99.5% (t_{R}=4.33 min).

### Step 8. N-(((1R,4R)-4-Hydroxy-1-(thiophen-3-yl)cyclohexyl)methyl)-2-methoxybenzamide

Note: Hₐ corresponds to axial protons and Hₑ to equatorial protons.

Synthesized from 2-methoxy-N-((4-oxo-1-(thiophen-3-yl)cyclohexyl)methyl)benzamide (5.90 g, 17.0 mmol, 1.0 equiv) and NaBH₄ (1.29 g, 34.0 mmol, 2.0 equiv) via general procedure H. Column chromatography, DCM/diethyl ether=2/1 (v/v). Yield: 34% (2.0 g); white solid. ¹H NMR (400 MHz, DMSO-d₆): δ_{H} 1.10-1.25 (2H, m, Hₐ-3,5), 1.45-1.57 (2H, m, Hₐ-2,6), 1.62-1.75 (2H, m, Hₑ-3,5), 2.10-2.20 (2H, m, Hₑ-2,6), 3.38 (2H, d, J=5.7 Hz, H-7′, H-7″), 3.42-3.51 (1H, m, H-4), 3.78 (3H, s, OCH₃), 4.40 (1H, d, J=4.5 Hz, OH), 7.02 (1H, td, J₁=7.7 Hz, J₂=1.0 Hz, H-13), 7.11 (1H, dd, J₁=8.4 Hz, J₂=0.9 Hz, H-11), 7.17 (1H, dd, J₁=5.0 Hz, J₂=1.4 Hz, H-18), 7.37 (1H, dd, J₁=2.9 Hz, J₂=1.4 Hz, H-16), 7.46 (1H, ddd, J₂=8.3 Hz, J₂=7.3 Hz, J₃=1.9 Hz, H-12), 7.57 (1H, dd, J₁=5.0 Hz, J₂=2.9 Hz, H-19), 7.69 (1H, brt, J=5.7 Hz, NHCO), 7.84 (1H, dd, J₁=7.7 Hz, J₂=1.9 Hz, H-14). ¹³C NMR (101 MHz, DMSO-d₆): δ_{C} 31.02 (C-3,5), 31.66 (C-2,6), 40.77 (C-1), 50.55 (C-7), 55.88 (C-15), 68.43 (C-4), 112.08 (C-11), 120.65 (C-13), 121.65 (C-16), 121.71 (C-9), 126.21 (C-19), 126.77 (C-18), 130.90 (C-14), 132.52 (C-12), 145.78 (C-17), 157.06 (C-10), 164.27 (C-8). HRMS (ESI+): m/z calcd for [M+H]⁺ 346.1471; found 346.1459. HPLC purity, 98.2% (t_{R}=3.84 min).

### Step 9. (3-Iodopropyl)triphenylphosphonium iodide

Synthesized from triphenylphosphine (3.02 g, 11.5 mmol, 1.0 equiv) and 1,3-diiodopropane (2.33 mL, 23 mmol, 2.0 equiv) via general procedure J. The product was used without further purification. Yield: 99.0% (6.36 g); white crystals. ¹H NMR (400 MHz, CDCl₃): δ 2.14-2.26 (m, 2H), 3.63 (td, J₁=6.4 Hz, J₂=1.5 Hz, 2H), 3.94-4.04 (m, 2H), 7.67-7.77 (m, 6H), 7.79-7.89 (m, 9H). HRMS (ESI+): m/z calcd for [M+H]⁺ 431.04201; found 431.04038.

### Step 10. (3-Azidopropyl)triphenylphosphonium iodide

Synthesized from (3-iodopropyl)triphenylphosphonium iodide (3.01 g, 5.4 mmol, 1.0 equiv) and sodium azide (0.70 g, 10.8 mmol, 2.0 equiv) via general procedure K. The product was used without further purification. Yield: 95.9% (2.45 g); white crystals. ¹H NMR (400 MHz, CDCl₃): δ 1.86-1.97 (m, 2H), 3.84 (td, J₂=6.3 Hz, J₂=1.0 Hz, 2H), 3.87-3.97 (m, 2H), 7.67-7.76 (m, 6H), 7.78-7.89 (m, 9H). HRMS (ESI+): m/z calcd for [M+H]⁺ 346.14676; found 346.14548.

### Step 11. (3-Aminopropyl)triphenylphosphonium iodide

Synthesized from (3-azidopropyl)triphenylphosphonium iodide (2.45 g, 5.2 mmol, 1.0 equiv) and Pd/C (0.25 g) via general procedure L. The product was used without further purification. Yield: 94.6% (2.20 g); white crystals. ¹H NMR (400 MHz, CDCl₃): δ 1.61 (s, 2H), 1.78 (ddd, J₁=8.0 Hz, J₂=5.1 Hz, J₃=1.6 Hz, 2H), 3.03 (t, J=6.2 Hz, 2H), 3.68-3.83 (m, 2H), 7.67-7.75 (m, 6H), 7.77-7.87 (m, 9H). HRMS (ESI+): m/z calcd for [M+H]⁺ 320.15626; found 320.15508.

### Step 12. (3-(((((1R,4R)-4-((2-Methoxybenzamido)methyl)-4-(thiophen-3-yl)cyclohexyl)oxy)carbonyl)amino)propyl)triphenylphosphonium iodide

Synthesized from N-(((1R,4R)-4-hydroxy-1-(thiophen-3-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (3-aminopropyl)triphenylphosphonium iodide (157 mg, 0.35 mmol, 1.2 equiv) according to general procedure I. Column chromatography, DCM/MeOH=100/1 (v/v). Two confomers in a 11:89 ratio. Yield: 24% (58 mg); white solid. ¹H NMR (400 MHz, DMSO) for both confomers: δ 7.94-7.86 (3H, m, H-27, H-33, H-39), 7.82 (1H, dd, J₁=7.7 Hz, J₂=1.8 Hz, H-14), 7.80-7.67 (13H, m, H-25, H-26, H-28, H-29, H-31, H-32, H-34, H-35, H-37, H-38, H-40, H-41, CH₂NHCO), 7.59 (1H, dd, J₁=4.9 Hz, J₂=2.9 Hz, H-19), 7.50-7.43 (1H, m, H-12), 7.39 (1H, d, J=1.5 Hz, H-16), 7.22-7.16 (1H, m, H-18), 7.10 (2H, t, J=6.4 Hz, H-11, OCONHCH₂), 7.05-6.98 (1H, m, H-13), 4.58-4.46 (1H, m, H-4), 3.77 (3H, s, OCH₃), 3.61-3.47 (2H, m, H-23′, H-23″), 3.43 (2H, d, J=5.6 Hz, H-7′, H-7″), 3.10 (2H, dd, J₁=12.3 Hz, J₂=6.2 Hz, H-21′, H-21″), 2.21-2.06 (2H, m, Hₑ-2,6), 1.85-1.72 (2H, m, Hₑ-3,5), 1.70-1.53 (4H, m, Hₐ-2,6, H-22′, H-22″), 1.29 (2H, dt, J₁=23.7 Hz, J₂=11.7 Hz, Hₐ-3,5); ¹³C NMR (101 MHz, DMSO) for both confomers: δ 164.39 (C-8), 157.03 (C-10), 155.68 (C-20), 145.62 (C-17), 134.98 (d, J=2.8 Hz, C-27, C-33, C-39), 133.55 (d, J=10.1 Hz, C-26, C-28, C-32, C-34, C-38, C-40), 132.53 (C-12), 130.84 (C-14), 130.26 (d, J=12.4 Hz, C-25, C-29, C-31, C-35, C-37, C-41), 126.67 (C-18), 126.42 (C-19), 121.79 (C-16), 121.70 (C-9), 120.66 (C-13), 118.31 (d, J=86.0 Hz, C-24, C-30, C-36), 112.08 (C-11), 71.59 (C-4), 55.87 (C-15), 49.68 (C-7), 40.63 (C-21), 40.46 (C-1), 30.99 (C-2,6), 27.37 (C-3,5), 22.41 (C-22), 18.18 (d, J=51.9 Hz, C-23); HRMS (ESI+): m/z calcd for [M+H]⁺ 691.2754; found 691.2745. HPLC purity, 97.28% at 254 nm (t_{R}=4.667 min).

### Example 2

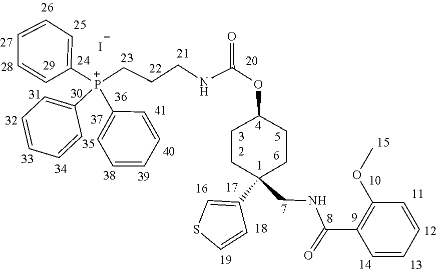 [SMILES: COc1ccccc1C(=O)NC[C@]1(c2ccsc2)CC[C@H](OC(=O)NCCC[P+](c2ccccc2)(c2ccccc2)c2ccccc2)CC1 ; [I-]]

### Steps 1, 2, 3, 4, 5, 6, and 7, are the Same as Steps 1, 2, 3, 4, 5, 6, and 7, for Example 1

### Step 8. N-(((1S,4S)-4-hydroxy-1-(thiophen-3-yl)cyclohexyl)methyl)-2-methoxybenzamide

Synthesized from 2-methoxy-N-((4-oxo-1-(thiophen-3-yl)cyclohexyl)methyl)benzamide (5.90 g, 17.0 mmol, 1.0 equiv) and NaBH₄ (1.29 g, 34.0 mmol, 2.0 equiv) via general procedure H. Column chromatography, DCM/diethyl ether=2/1 (v/v). Yield: 57% (3.3 g); white solid. ¹H NMR (400 MHz, DMSO-d₆): δ_{H} 1.46-1.61 (4H, m, Hₐ-3,5, Hₑ-3,5), 1.62-1.72 (2H, m, Hₐ-2,6), 1.89-1.99 (2H, m, Hₑ-2,6), 3.46-3.54 (1H, m, H-4), 3.59 (2H, d, J=5.8 Hz, H-7′, H-7″), 3.71 (3H, s, OCH₃), 4.50 (1H, d, J=4.0 Hz, OH), 7.03 (1H, td, J₁=7.5 Hz, J₂=1.0 Hz, H-13), 7.09 (1H, dd, J₁=8.4 Hz, J₂=1.0 Hz, H-11), 7.20 (1H, dd, J₁=5.0 Hz, J₂=1.4 Hz, H-18), 7.33 (1H, dd, J₁=2.9 Hz, J₂=1.4 Hz, H-16), 7.46 (1H, ddd, J₁=8.4 Hz, J₂=7.3 Hz, J₃=1.9 Hz, 1H, H-12), 7.57 (1H, dd, J₁=5.0 Hz, J₂=2.9 Hz, H-19), 7.62 (1H, brt, J=5.7 Hz, NHCO), 7.89 (1H, dd, J₂=7.8 Hz, J₂=1.9 Hz, H-14). ¹³C NMR (101 MHz, DMSO-d₆): δ_{C} 30.22 (C-3,5), 30.50 (C-2,6), 39.86 (C-1), 46.77 (C-7), 55.80 (C-15), 66.74 (C-4), 112.11 (C-11), 120.70 (C-13, C-16), 121.24 (C-9), 126.14 (C-19), 126.51 (C-18), 131.06 (C-14), 132.66 (C-12), 147.81 (C-17), 157.11 (C-10), 164.04 (C-8). HRMS (ESI+): m/z calcd for [M+H]⁺ 346.1471; found 346.1459. HPLC purity, 96.1% at 254 nm (t_{R}=4.31 min).

### Steps 9, 10, and 11 are the Same as Steps 9, 10, and 11 for Example 1

### Step 12. (3-(((((1S,4S)-4-((2-Methoxybenzamido)methyl)-4-(thiophen-3-yl)cyclohexyl)oxy)carbonyl)amino)propyl)triphenylphosphonium iodide

Synthesized from N-(((1S,4S)-4-hydroxy-1-(thiophen-3-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (3-aminopropyl)triphenylphosphonium iodide (157 mg, 0.35 mmol, 1.2 equiv) according to general procedure I. Column chromatography, DCM/MeOH=100/1 (v/v). Two confomers in a 11:89 ratio. Yield: 25% (60 mg); white solid. ¹H NMR (400 MHz, DMSO) for both confomers: δ 7.94-7.85 (4H, m, H-14, H-27, H-33, H-39), 7.84-7.73 (12H, m, H-25, H-26, H-28, H-29, H-31, H-32, H-34, H-35, H-37, H-38, H-40, H-41), 7.67-7.61 (1H, m, CH₂NHCO), 7.60 (1H, dd, J₁=5.0 Hz, J₂=2.9 Hz, H-19), 7.50-7.41 (1H, m, H-12), 7.37 (1H, d, J=1.5 Hz, H-16), 7.26 (1H, t, J=5.6 Hz, OCONHCH₂), 7.22 (1H, dd, J₁=5.0 Hz, J₂=1.2 Hz, H-18), 7.09 (1H, d, J=8.2 Hz, H-11), 7.03 (1H, t, J=7.5 Hz, H-13), 4.66-4.40 (1H, m, H-4), 3.70 (3H, s, OCH₃), 3.63-3.44 (4H, m, H-7′, H-7″, H-23′, H-23″), 3.15 (2H, dd, J₁=12.5 Hz, J₂=6.4 Hz, H-21′, H-21″), 1.96-1.74 (4H, m, Hₐ-2,6, Hₑ-2,6), 1.76-1.45 (6H, m, Hₐ-3,5, Hₑ-3,5, H-22′, H-22″); ¹³C NMR (101 MHz, CDCl₃) for both confomers: δ 165.18 (C-8), 157.57 (C-10), 156.75 (C-20), 146.87 (C-17), 135.28 (d, J=2.8 Hz, C-27, C-33, C-39), 133.76 (d, J=10.1 Hz, C-26, C-28, C-32, C-34, C-38, C-40), 132.77 (C-12), 132.42 (C-14), 130.70 (d, J=12.5 Hz, C-25, C-29, C-31, C-35, C-37, C-41), 126.48 (C-18), 125.92 (C-19), 121.33 (C-9), 121.21 (C-13), 120.86 (C-16), 118.19 (d, J=86.4 Hz, C-24, C-30, C-36), 111.35 (C-11), 71.50 (C-4), 55.85 (C-15), 48.76 (C-7), 40.78 (C-21), 40.36 (C-1), 30.57 (C-2,6), 27.11 (C-3,5), 23.15 (C-22), 21.02 (d, J=52.7 Hz, C-23); HRMS (ESI+): m/z calcd for [M+H]⁺ 691.2754; found 691.2732. HPLC purity, 96.94% at 254 nm (t_{R}=4.873 min).

### Example 3

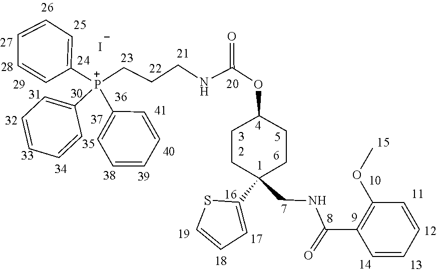 [SMILES: COc1ccccc1C(=O)NC[C@]1(c2cccs2)CC[C@H](OC(=O)NCCC[P+](c2ccccc2)(c2ccccc2)c2ccccc2)CC1 ; [I-]]

### Step 1. Dimethyl 4-cyano-4-(thiophen-2-yl)heptanedioate

Synthesized from 2-(thiophen-2-yl)acetonitrile (10.60 mL, 100.0 mmol, 1.0 equiv), methyl acrylate (45.30 mL, 500.0 mmol, 5.0 equiv) and benzyltrimethylammonium hydroxide (17.60 mL, 100.0 mmol, 1.0 equiv.) via general procedure A. The product was used without further purification. Yield: 80% (24.00 g); pale yellow oil. ¹H NMR (400 MHz, CDCl₃): δ 2.20-2.33 (m, 4H), 2.34-2.45 (m, 2H), 2.49-2.60 (m, 2H), 3.65 (s, 6H), 6.97 (dd, J₁=5.1 Hz, J₂=3.6 Hz, 1H), 7.13 (dd, J₁=3.6 Hz, J₂=1.2 Hz, 1H), 7.32 (dd, J₁=5.1 Hz, J₂=1.2 Hz, 1H).

### Step 2. Methyl 5-cyano-2-oxo-5-(thiophen-2-yl)cyclohexane-1-carboxylate

Synthesized from Dimethyl 4-cyano-4-(thiophen-2-yl)heptanedioate (24.00 g, 80.0 mmol, 1.0 equiv) and potassium tert-butoxide (17.95 g, 160.0 mmol, 2 equiv) via general procedure B. The product was used without further purification. Yield: 60% (12.60 g); pale yellow solid. ¹H NMR (400 MHz, DMSO): δ 2.22-2.31 (m, 1H), 2.39-2.49 (m, 2H), 2.52-2.67 (m, 2H), 2.77 (dd, J₁=15.7 Hz, J₂=1.0 Hz, 1H), 3.03 (d, J=16.1 Hz, 1H), 3.76 (s, 3H), 7.08 (dd, J₁=5.1 Hz, J₂=3.6 Hz, 1H), 7.27 (dd, J₁=3.6 Hz, J₂=1.2 Hz, 1H), 7.59 (dd, J₁=5.1 Hz, J₂=1.2 Hz, 1H). HRMS (ESI+): m/z calcd for [M+H]⁺ 264.0689; found 264.0682.

### Step 3. 4-Oxo-1-(thiophen-2-yl)cyclohexane-1-carbonitrile

Synthesized from Methyl 5-cyano-2-oxo-5-(thiophen-2-yl)cyclohexane-1-carboxylate (12.64 g, 48.0 mmol, 1.0 equiv), 10% sulfuric acid (175 mL) and glacial acetic acid (385 mL) via general procedure C. Column chromatography, EtOAc/n-hex=1/3 (v/v). Yield: 50% (4.90 g); pale yellow solid. ¹H NMR (400 MHz, CDCl₃): δ 2.31 (td, J=13.5 Hz, J₂=4.3 Hz, 2H), 2.52-2.61 (m, 2H), 2.61-2.69 (m, 2H), 2.80-2.92 (m, 2H), 7.03 (dd, J₁=5.1 Hz, J₂=3.6 Hz, 1H), 7.20 (dd, J₁=3.6 Hz, J₂=1.2 Hz, 1H), 7.33 (dd, J₁=5.1 Hz, J₂=1.2 Hz, 1H). HRMS (ESI+): m/z calcd for [M+H]⁺ 206.0634; found 206.0629.

### Step 4. 8-(Thiophen-2-yl)-1,4-dioxaspiro[4.5]decane-8-carbonitrile

Synthesized from 4-oxo-1-(thiophen-2-yl)cyclohexane-1-carbonitrile (4.93 g, 24.0 mmol, 1.0 equiv), ethane-1,2-diol (13.4 mL, 240.0 mmol, 10.0 equiv) and p-toluenesulfonic acid (86.10 mg, 0.5 mmol, 0.02 equiv.) via general procedure D. Column chromatography, EtOAc/n-hex=1/3 (v/v). Yield: 98% (5.90 g); white solid. ¹H NMR (400 MHz, CDCl₃): δ 1.82-1.90 (m, 2H), 2.06 (td, J₁=13.4 Hz, J₂=4.0 Hz, 2H), 2.19 (td, J₁=13.2 Hz, J₂=3.5 Hz, 2H), 2.28-2.38 (m, 2H), 3.93-3.98 (m, 2H), 3.98-4.03 (m, 2H), 6.99 (dd, J₁=5.1 Hz, J₂=3.6 Hz, 1H), 7.15 (dd, J₁=3.6 Hz, J₂=1.2 Hz, 1H), 7.27 (dd, J₁=5.2 Hz, J₂=1.3 Hz, 1H). HRMS (ESI+): m/z calcd for [M+H]⁺ 250.0896; found 250.0890.

### Step 5. (8-(Thiophen-2-yl)-1,4-dioxaspiro[4.5]decan-8-yl)methanamine

Synthesized from 8-(thiophen-2-yl)-1,4-dioxaspiro[4.5]decane-8-carbonitrile (5.90 g, 23.5 mmol, 1.0 equiv) and LiAlH₄ (1.78 g, 47.0 mmol, 2.0 equiv) via general procedure E. The product was used without further purification. Yield: 97% (5.80 g); pale yellow oil. Yield: 97% (5.8 g); oil. ¹H NMR (400 MHz, CDCl₃): δ 0.95 (brs, 2H), 1.63-1.74 (m, 4H), 1.75-1.85 (m, 2H), 2.10-2.18 (m, 2H), 2.72 (s, 2H), 3.89-3.98 (m, 4H), 6.86 (dd, J₁=3.5 Hz, J₂=1.1 Hz, 1H), 6.97 (dd, J₁=5.1 Hz, J₂=3.5 Hz, 1H), 7.21 (dd, J₁=5.1 Hz, J₂=1.1 Hz, 1H). HRMS (ESI+): m/z calcd for [M+H]⁺ 254.1209; found 254.1201.

### Step 6. 2-Methoxy-N-((8-(thiophen-2-yl)-1,4-dioxaspiro[4.5]decan-8-yl)methyl)benzamide

Synthesized from (8-(thiophen-2-yl)-1,4-dioxaspiro[4.5]decan-8-yl)methanamine (5.80 g, 22.8 mmol, 1.0 equiv), 2-methoxybenzoyl chloride (3.89 g, 22.8 mmol, 1 equiv) and Et₃N (9.5 mL, 68.4 mmol, 3.0 equiv.) via general procedure F. Column chromatography, EtOAc/n-hex=1/1 (v/v). Yield: 95% (8.40 g); white solid. ¹H NMR (400 MHz, CDCl₃): δ 1.65-1.80 (m, 4H), 1.95-2.05 (m, 2H), 2.12-2.20 (m, 2H), 3.68 (d, J=6.0 Hz, 2H), 3.73 (s, 3H), 3.87-3.98 (m, 4H), 6.90 (d, J=7.8 Hz, 1H), 6.95 (dd, J₁=3.5 Hz, J₂=1.1 Hz, 1H), 7.03 (ddd, J₁=9.5 Hz, J₂=6.6 Hz, J₃=2.2 Hz, 2H), 7.27 (dd, J₁=5.1 Hz, J₂=1.0 Hz, 1H), 7.40 (ddd, J₁=8.3 Hz, J₂=7.3 Hz, J₃=1.9 Hz, 1H), 7.77 (t, J=4.9 Hz, 1H), 8.20 (dd, J₁=7.8 Hz, J₂=1.9 Hz, 1H). HRMS (ESI+): m/z calcd for [M+H]⁺ 388.1577; found 388.1565.

### Step 7. 2-Methoxy-N-((4-oxo-1-(thiophen-2-yl)cyclohexyl)methyl)benzamide

Synthesized from 2-methoxy-N-((8-(thiophen-2-yl)-1,4-dioxaspiro[4.5]decan-8-yl)methyl)benzamide (8.40 g, 21.7 mmol, 1.0 equiv), pyridinium p-toluenesulfonate (0.55 g, 2.2 mmol, 0.1 equiv) and water (20 mL) via general procedure G. Column chromatography, EtOAc/n-hex=1/1 (v/v). Yield: 75% (5.50 g); white solid. ¹H NMR (400 MHz, CDCl₃): δ 2.12-2.24 (2H, m, Hₐ-2,6), 2.37-2.53 (6H, m, Hₐ-3,5, Hₑ-2,6, Hₑ-3,5), 3.75 (3H, s, OCH₃), 3.80 (2H, d, J=6.2 Hz, H-7′, H-7″), 6.92 (1H, d, J=8.3 Hz, H-11), 7.03-7.08 (2H, m, H-17, H-13), 7.09 (1H, dd, J₁=5.1 Hz, J₂=3.6 Hz, H-18), 7.35 (1H, dd, J₁=5.1 Hz, J₂=1.1 Hz, H-19), 7.43 (1H, ddd, J₁=8.4 Hz, J₂=7.3 Hz, J₃=1.9 Hz, H-12), 7.88 (1H, t, J=5.3 Hz, NHCO), 8.20 (1H, dd, J₁=7.8 Hz, J₂=1.8 Hz, H-14). ¹³C NMR (100 MHz, CDCl₃): δ 35.09 (C-2,6), 37.70 (C-3,5), 42.09 (C-1), 50.70 (C-7), 55.65 (C-15), 111.27 (C-11), 121.03 (C-9), 121.40 (C-13), 124.75 (C-19), 124.91 (C-17), 127.17 (C-18), 132.54 (C-14), 133.03 (C-12), 148.47 (C-16), 157.55 (C-10), 165.48 (C-8), 210.80 (C-4). HRMS (ESI+): m/z calcd for [M+H]⁺ 344.1315; found 344.1304. HPLC purity, 98.8% at 254 nm (t_{R}=4.41 min).

### Step 8. N-(((1R,4R)-4-Hydroxy-1-(thiophen-2-yl)cyclohexyl)methyl)-2-methoxybenzamide

Synthesized from 2-methoxy-N-((4-oxo-1-(thiophen-2-yl)cyclohexyl)methyl)benzamide (5.50 g, 15.9 mmol, 1.0 equiv) and NaBH₄ (1.20 g, 31.8 mmol, 2.0 equiv) via general procedure H. Column chromatography, DCM/diethyl ether=2/1 (v/v). Yield: 43% (2.4 g); white solid. ¹H NMR (400 MHz, DMSO-d₆): δ_{H} 1.50-1.65 (4H, m, Hₑ-3,5, Hₐ-3,5), 1.67-1.76 (2H, m, Hₐ-2,6), 1.93-2.03 (2H, m, Hₑ-2,6), 3.51-3.61 (3H, m, H-4, H-7′, H-7″), 3.73 (3H, s, OCH₃), 4.53 (1H, d, J=3.7 Hz, OH), 6.99-7.09 (3H, m, H-17, H-13, H-18), 7.11 (1H, d, J=8.3 Hz, H-11), 7.44-7.49 (2H, m, H-12, H-19), 7.81 (1H, brt, J=5.9 Hz, NHCO), 7.89 (1H, d, J=7.8 Hz, H-14). ¹³C NMR (101 MHz, DMSO-d₆): δ_{C} 30.00 (C-3,5), 31.28 (C-2,6), 41.04 (C-1), 48.56 (C-7), 55.80 (C-15), 66.09 (C-4), 112.15 (C-11), 120.71 (C-13), 121.38 (C-9), 123.92 (C-19), 124.08 (C-17), 126.87 (C-18), 131.02 (C-14), 132.68 (C-12), 151.28 (C-16), 157.12 (C-10), 164.20 (C-8). HRMS (ESI+): m/z calcd for [M+H]⁺ 346.1471; found 346.1457. HPLC purity, 96.1% at 254 nm (t_{R}=4.36 min).

### Steps 9, 10, and 11 are the Same as Steps 9, 10, and 11 for Example 1

### Step 12. (3-(((((1R,4R)-4-((2-Methoxybenzamido)methyl)-4-(thiophen-2-yl)cyclohexyl)oxy)carbonyl)amino)propyl)triphenylphosphonium iodide

Synthesized from N-(((1R,4R)-4-hydroxy-1-(thiophen-2-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (3-aminopropyl)triphenylphosphonium iodide (157 mg, 0.35 mmol, 1.2 equiv) according to general procedure I. Column chromatography, DCM/MeOH=100/1 (v/v). Two confomers in a 18:82 ratio. Yield: 26% (62 mg); white solid. ¹H NMR (400 MHz, DMSO) for both confomers: δ 7.95-7.88 (3H, m, H-27, H-33, H-39), 7.87 (1H, dd, J₁=8.4 Hz, J₂=2.2 Hz, H-14), 7.84-7.74 (13H, m, H-25, H-26, H-28, H-29, H-31, H-32, H-34, H-35, H-37, H-38, H-40, H-41, CH₂NHCO), 7.51 (1H, dd, J₁=5.1 Hz, J₂=1.0 Hz, H-19), 7.50-7.42 (1H, m, H-12), 7.28 (1H, t, J=5.6 Hz, OCONHCH₂), 7.15-7.07 (2H, m, H-11, H-18), 7.06-6.98 (2H, m, H-17, H-13), 4.65-4.50 (1H, m, H-4), 3.72 (3H, s, OCH₃), 3.59 (2H, d, J=5.2 Hz, H-7′, H-7″), 3.66-3.45 (2H, m, H-23′, H-23″), 3.16 (2H, q, J=6.5 Hz, H-21′, H-21″), 1.99-1.77 (4H, m, Hₑ-2,6, Hₐ-2,6), 1.77-1.48 (6H, m, Hₑ-3,5, Hₐ-3,5, H-22′, H-22″); ¹³C NMR (101 MHz, DMSO) for both confomers: δ 164.31 (C-8), 157.14 (C-10), 155.73 (C-20), 150.14 (C-16), 134.99 (d, J=2.7 Hz, C-27, C-33, C-39), 133.58 (d, J=10.2 Hz, C-26, C-28, C-32, C-34, C-38, C-40), 132.72 (C-12), 130.99 (C-14), 130.29 (d, J=12.5 Hz, C-25, C-29, C-31, C-35, C-37, C-41), 127.03 (C-18), 124.41 (C-17), 124.32 (C-19), 121.24 (C-9), 120.71 (C-13), 118.33 (d, J=86.2 Hz, C-24, C-30, C-36), 112.19 (C-11), 69.77 (C-4), 55.83 (C-15), 49.06 (C-7), 41.06 (C-21), 40.34 (C-1), 31.04 (C-2,6), 26.70 (C-3,5), 22.42 (C-22), 18.24 (d, J=52.2 Hz, C-23); HRMS (ESI+): m/z calcd for [M+H]⁺ 691.2754; found 691.2733. HPLC purity, 97.46% at 254 nm (t_{R}=4.910 min).

### Example 4

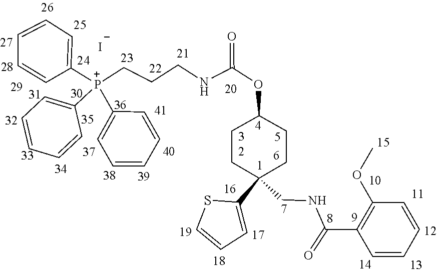 [SMILES: COc1ccccc1C(=O)NC[C@]1(c2cccs2)CC[C@@H](OC(=O)NCCC[P+](c2ccccc2)(c2ccccc2)c2ccccc2)CC1 ; [I-]]

### Steps 1, 2, 3, 4, 5, 6, and 7 are the Same as Steps 1, 2, 3, 4, 5, 6, and 7 for Example 3

### Step 8. N-(((1S,4S)-4-Hydroxy-1-(thiophen-2-yl)cyclohexyl)methyl)-2-methoxybenzamide

Synthesized from 2-methoxy-N-((4-oxo-1-(thiophen-2-yl)cyclohexyl)methyl)benzamide (5.50 g, 15.9 mmol, 1.0 equiv) and NaBH₄ (1.20 g, 31.8 mmol, 2.0 equiv) via general procedure H. Column chromatography, DCM/diethyl ether=2/1 (v/v). Yield: 31% (1.7 g); white solid. ¹H NMR (400 MHz, DMSO-d₆): δ_{H} 1.20-1.32 (2H, m, Hₐ-3,5), 1.57-1.67 (2H, m, Hₐ-2,6), 1.68-1.75 (2H, m, Hₑ-3,5), 2.05-2.13 (2H, m, Hₑ-2,6), 3.41 (2H, d, J=6.0 Hz, H-7′, H-7″), 3.41-3.51 (1H, m, H-4), 3.78 (3H, s, OCH₃), 4.45 (1H, d, J=4.6 Hz, OH), 7.01 (1H, dd, J₁=3.4 Hz, J₂=1.2 Hz, H-17), 7.01-7.05 (1H, m, H-13), 7.08 (1H, dd, J₁=5.1 Hz, J₂=3.5 Hz, H-18), 7.12 (1H, dd, J₁=8.4 Hz, J₂=1.0 Hz, H-11), 7.43-7.47 (1H, m, H-12), 7.48 (1H, dd, J₁=5.1 Hz, J₂=1.1 Hz, H-19), 7.82 (1H, dd,J₁=7.7 Hz,J₂=1.8 Hz, H-14), 7.85 (1H, brt,J=6.0 Hz, NHCO). ¹³C NMR (101 MHz, DMSO-d₆): δ_{C} 30.95 (C-3,5), 32.58 (C-2,6), 41.74 (C-1), 51.71 (C-7), 55.86 (C-15), 68.10 (C-4), 112.11 (C-11), 120.65 (C-13), 121.87 (C-9), 124.42 (C-19), 124.61 (C-17), 127.09 (C-18), 130.83 (C-14), 132.52 (C-12), 149.72 (C-16), 157.04 (C-10), 164.44 (C-8). HRMS (ESI+): m/z calcd for [M+H]⁺ 346.1471; found 346.1457. HPLC purity, 96.6% at 254 nm (t_{R}=4.00 min).

### Steps 9, 10, and 11 are the Same as Steps 9, 10, and 11 for Example 1

### Step 12. (3-(((((1S,4S)-4-((2-Methoxybenzamido)methyl)-4-(thiophen-2-yl)cyclohexyl)oxy)carbonyl)amino)propyl)triphenylphosphonium iodide

Synthesized from N-(((1S,4S)-4-hydroxy-1-(thiophen-2-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (3-aminopropyl)triphenylphosphonium iodide (157 mg, 0.35 mmol, 1.2 equiv) according to general procedure I. Column chromatography, DCM/MeOH=100/1 (v/v). Two confomers in a 12:88 ratio. Yield: 23% (55 mg); white solid. ¹H NMR (400 MHz, DMSO) for both confomers: δ 7.94-7.86 (4H, m, H-27, H-33, H-39, CH₂NHCO), 7.81 (1H, dd, J₁=7.7 Hz, J₂=1.8 Hz, H-14), 7.79-7.69 (12H, m, H-25, H-26, H-28, H-29, H-31, H-32, H-34, H-35, H-37, H-38, H-40, H-41), 7.54-7.44 (2H, m, H-19, H-12), 7.16-7.07 (3H, H-11, OCONHCH₂, H-18), 7.06-7.00 (2H, m, H-17, H-13), 4.58-4.45 (1H, m, H-4), 3.78 (3H, s, OCH₃), 3.54 (2H, t, J=14.8 Hz, H-23′, H-23″), 3.45 (2H, d, J=5.9 Hz, H-7′, H-7″), 3.11 (2H, dd, J₂=12.3 Hz, J₂=6.3 Hz, H-21′, H-21″), 2.16-2.02 (2H, m, Hₑ-2,6), 1.89-1.55 (6H, m, Hₐ-2,6, Hₑ-3,5, H-22′, H-22″), 1.47-1.21 (2H, m, Hₐ-3,5); ¹³C NMR (101 MHz, DMSO) δ 164.57 (C-8), 157.00 (C-10), 155.65 (C-20), 149.40 (C-16), 134.97 (d, J=2.8 Hz, C-27, C-33, C-39), 133.55 (d, J=10.1 Hz, C-26, C-28, C-32, C-34, C-38, C-40), 132.51 (C-12), 130.75 (C-14), 130.26 (d, J=12.5 Hz, C-25, C-29, C-31, C-35, C-37, C-41), 127.11 (C-18), 124.67 (C-17), 124.56 (C-19), 121.97 (C-9), 120.65 (C-13), 118.31 (d, J=86.0 Hz, C-24, C-30, C-36), 112.11 (C-11), 71.32 (C-4), 55.85 (C-15), 50.85 (C-7), 41.59 (C-21), 40.47 (C-1), 31.95 (C-2,6), 27.33 (C-3,5), 22.38 (d, J=4.2 Hz, C-22), 18.19 (d, J=51.8 Hz, C-23); HRMS (ESI+): m/z calcd for [M+H]⁺ 691.2754; found 691.2742. HPLC purity, 98.77% at 254 nm (t_{R}=4.697 min).

### Example 5

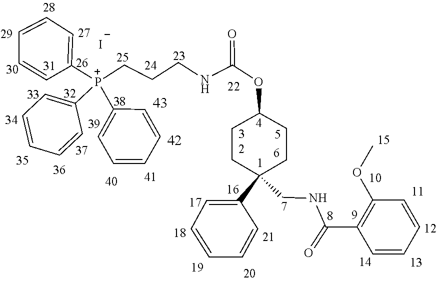 [SMILES: COc1ccccc1C(=O)NC[C@]1(c2ccccc2)CC[C@@H](OC(=O)NCCC[P+](c2ccccc2)(c2ccccc2)c2ccccc2)CC1 ; [I-]]

### Step 1. 8-Phenyl-1,4-dioxaspiro[4.5]decane-8-carbonitrile

Synthesized from 4-oxo-1-phenylcyclohexane-1-carbonitrile (5.00 g, 25.1 mmol, 1.0 equiv), ethane-1,2-diol (14.0 mL, 251.0 mmol, 1.0 equiv) and p-toluenesulfonic acid (86 mg, 0.5 mmol, 0.2 equiv) via general procedure D. Column chromatography, EtOAc/n-hexane=1/4 (v/v). Yield: 95% (5.80 g); white solid. ¹H NMR (400 MHz, CDCl₃): δ 1.84-1.91 (m, 2H), 2.07-2.24 (m, 6H), 3.94-3.99 (m, 2H), 3.99-4.04 (m, 2H), 7.32 (ddd, J₁=7.2 Hz, J₂=3.7 Hz, J₃=1.2 Hz, 1H), 7.36-7.43 (m, 2H), 7.49-7.55 (m, 2H). HRMS (ESI+): m/z calcd for [M+H]⁺ 244.1332; found 244.1326.

### Step 2. 8-Phenyl-1,4-dioxaspiro[4.5]decan-8-yl)methanamine

Synthesized from 8-phenyl-1,4-dioxaspiro[4.5]decane-8-carbonitrile (5.80 g, 23.8 mmol, 1 equiv) and LiAlH₄ (1.81 g, 47.6 mmol, 2.0 equiv) via general procedure E. The product was used without further purification. Yield: 96% (5.68 g); uncoloured oil. ¹H NMR (400 MHz, CDCl₃): δ 1.49-1.59 (m, 2H), 1.62-1.70 (m, 2H), 1.70-1.79 (m, 2H), 2.20-2.30 (m, 2H), 2.70 (s, 2H), 3.86-3.92 (m, 2H), 3.92-3.96 (m, 2H), 7.17-7.24 (m, 1H), 7.30-7.38 (m, 4H).

### Step 3. 2-Methoxy-N-((8-phenyl-1,4-dioxaspiro[4.5]decan-8-yl)methyl)benzamide

Synthesized from 2-methoxybenzoyl chloride (3.92 g, 22.95 mmol, 1.0 equiv), Et₃N (9.60 mL, 68.9 mmol, 3.0 equiv) and 8-phenyl-1,4-dioxaspiro[4.5]decan-8-yl)methanamine (5.68 g, 22.95 mmol, 1 equiv) via general procedure F. The product was used without further purification. Yield: 95% (8.32 g); white solid. ¹H NMR (400 MHz, CDCl₃): δ 1.52-1.62 (m, 2H), 1.71-1.80 (m, 2H), 1.90-2.01 (m, 2H), 2.20-2.29 (m, 2H), 3.61 (s, 3H), 3.67 (d, J=5.9 Hz, 2H), 3.86-3.91 (m, 2H), 3.91-3.96 (m, 2H), 6.86 (d, J=8.3 Hz, 1H), 7.00-7.06 (m, 1H), 7.23-7.30 (m, 1H), 7.35-7.46 (m, 5H), 7.58 (t, J=5.4 Hz, 1H), 8.18 (dd, J₁=7.8 Hz, J₂=1.9 Hz, 1H).

### Step 4. 2-Methoxy-N-((4-oxo-1-phenylcyclohexyl)methyl)benzamide

Synthesized from 2-methoxy-N-((8-phenyl-1,4-dioxaspiro[4.5]decan-8-yl)methyl)benzamide (4.16 g, 10.90 mmol, 1.0 equiv), pyridinium p-toluenesulfonate (274 mg, 1.09 mmol, 0.1 equiv) and water (15 mL) via general procedure G. Column chromatography, EtOAc/n-hex=1/1 (v/v). Yield: 79% (2.91 g); white solid. ¹H NMR (400 MHz, CDCl₃): δ 2.07-2.18 (2H, m, Hₐ-2,6), 2.25-2.37 (2H, m, Hₐ-3,5), 2.43-2.56 (4H, m, Hₑ-3,5, Hₑ-2,6), 3.64 (3H, s, OCH₃), 3.78 (2H, d, J=6.2 Hz, H-7′, H-7″), 6.89 (1H, d, J=8.3 Hz, H-11), 7.02-7.08 (1H, m, H-13), 7.31-7.37 (1H, m, H-19), 7.41 (1H, ddd, J₁=8.4 Hz, J₂=7.3 Hz, J₃=1.9 Hz, H-12), 7.44-7.53 (4H, m, H-17, H-18, H-20, H-21), 7.67 (1H, t, J=5.2 Hz, NHCO), 8.19 (1H, dd, J₁=7.8 Hz, J₂=1.8 Hz, H-14). ¹³C NMR (101 MHz, CDCl₃): δ 33.32 (C-2,6), 37.86 (C-3,5), 42.36 (C-1), 50.17 (C-7), 55.57 (C-15), 111.21 (C-11), 121.07 (C-9), 121.38 (C-13), 126.89 (C-18,20), 127.02 (C-19), 129.20 (C-17,21), 132.54 (C-14), 132.96 (C-12), 142.25 (C-16), 157.49 (C-10), 165.44 (C-8), 211.19 (C-4). HRMS (ESI+):m/z calcd for [M+H]⁺ 338.1751; found 338.1755. HPLC purity, 97.7% at 254 nm (t_{R}=4.48 min).

### Step 5. N-(((1R,4R)-4-Hydroxy-1-phenylcyclohexyl)methyl)-2-methoxybenzamide

Synthesized from 2-methoxy-N-((4-oxo-1-phenylcyclohexyl)methyl)benzamide (2.91 g, 8.63 mmol, 1 equiv) and NaBH₄ (0.66 g, 17.27 mmol, 2 equiv) via general procedure H. Column chromatography, DCM/diethyl ether=3/1 (v/v). Yield: 33% (0.97 g); white solid. ¹H NMR (400 MHz, DMSO-d₆): δ_{H} 1.00-1.24 (2H, m, Hₐ-3,5), 1.40-1.60 (2H, m, Hₐ-2,6), 1.61-1.75 (2H, m, Hₑ-3,5), 2.20-2.32 (2H, m, Hₑ-2,6), 3.39 (2H, d, J=5.8 Hz, H-7′, H-7″), 3.46-3.55 (1H, m, H-4), 3.70 (3H, s, OCH₃), 4.39 (1H, d, J=4.3 Hz, OH), 7.01 (1H, td, J₁=7.6 Hz, J₂=0.9 Hz, H-13), 7.09 (1H, dd, J₁=8.4 Hz, J₂=0.9 Hz, H-11), 7.23-7.30 (1H, m, H-19), 7.37-7.50 (5H, m, H-12, H-17, H-18, H-20, H-21), 7.66 (1H, brt, J=5.8 Hz, NHCO), 7.81 (1H, dd, J₁=7.6 Hz, J₂=1.9 Hz, H-14). ¹³C NMR (101 MHz, DMSO-d₆): δ_{C} 30.74 (C-2,6), 31.02 (C-3,5), 41.94 (C-1), 51.18 (C-7), 55.79 (C-15), 68.60 (C-4), 112.05 (C-11), 120.63 (C-13), 121.77 (C-9), 125.93 (C-19), 127.01 (C-18,20), 128.58 (C-17,21), 130.84 (C-14), 132.47 (C-12), 143.52 (C-16), 156.97 (C-10), 164.30 (C-8). HRMS (ESI+): m/z calcd for [M+H]⁺ 340.1907; found 340.1907. HPLC purity, 96.8% at 254 nm (t_{R}=4.09 min).

### Steps 6, 7, and 8 are the Same as Steps 9, 10, and 11 for Example 1

### Step 9. (3-(((((1R,4R)-4-((2-Methoxybenzamido)methyl)-4-phenylcyclohexyl)oxy)carbonyl)amino)propyl)triphenylphosphonium iodide

Synthesized from N-(((1R,4R)-4-hydroxy-1-phenylcyclohexyl)methyl)-2-methoxybenzamide (100 mg, 0.29 mmol, 1.0 equiv), 4-nitrophenyl chloroformate (119 mg, 0.58 mmol, 2 equiv), Et₃N (0.12 mL, 0.87 mmol, 3 equiv) and (3-aminopropyl)triphenylphosphonium iodide (158 mg, 0.35 mmol, 1.2 equiv) according to general procedure I. Column chromatography, DCM/MeOH=100/1 (v/v). Two confomers in a 11:89 ratio. Yield: 24% (57 mg); white solid. ¹H NMR (400 MHz, DMSO): δ¹H NMR (400 MHz, DMSO) for both confomers: δ 7.95-7.84 (3H, m, H-29, H-35, H-41), 7.83-7.64 (14H, m, CH₂NHCO, H-14, H-27, H-28, H-30, H-31, H-33, H-34, H-36, H-37, H-39, H-40, H-42, H-43), 7.51-7.38 (5H, m, H-12, H-17, H-18, H-20, H-21), 7.28 (1H, t, J=6.7 Hz, H-19), 7.13-7.05 (2H, m, H-11, OCONHCH₂), 7.04-6.97 (1H, m, H-13), 4.61-4.49 (1H, m, H-4), 3.69 (3H, s, OCH₃), 3.52 (2H, tt, J₁=13.2 Hz, J₂=6.4 Hz, H-25′, H-25″), 3.45 (2H, d, J=5.6 Hz, H-7′, H-7″), 3.09 (2H, dd, J₁=12.2 Hz, J₂=6.2 Hz, H-23′, H-23″), 2.29-2.18 (2H, m, Hₑ-2,6), 1.86-1.73 (2H, m, Hₑ-3,5), 1.71-1.53 (4H, m, Hₐ-2,6, H-24′, H-24″), 1.34-1.12 (2H, m, Hₐ-3,5); ¹³C NMR (101 MHz, DMSO) for both confomers: δ 164.42 (C-8), 156.95 (C-10), 155.66 (C-22), 143.39 (C-16), 134.96 (d, J=2.5 Hz, C-29, C-35, C-41), 133.54 (d, J=10.1 Hz, C-28, C-30, C-34, C-36, C-40, C-42), 132.49 (C-12), 130.77 (C-14), 130.25 (d, J=12.4 Hz, C-27, C-31, C-33, C-37, C-39, C-43), 128.62 (C-18, C-20), 126.87 (C-17, C-21), 126.12 (C-19), 121.83 (C-9), 120.64 (C-13), 118.30 (d, J=86.0 Hz, C-26, C-32, C-38), 112.06 (C-11), 71.70 (C-4), 55.78 (C-15), 50.18 (C-7), 41.79 (C-1), 40.44 (C-23), 30.10 (C-2,6), 27.35 (C-3,5), 22.37 (d, J=3.4 Hz, C-24), 18.18 (d, J=51.9 Hz, C-25); HRMS (ESI+): m/z calcd for [M+H]⁺ 685.3190; found 685.3183. HPLC purity, 97.97% at 254 nm (t_{R}=4.740 min).

### Example 6

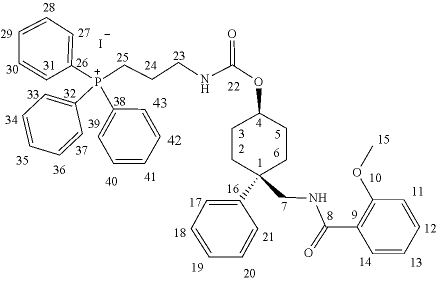 [SMILES: COc1ccccc1C(=O)NC[C@]1(c2ccccc2)CC[C@H](OC(=O)NCCC[P+](c2ccccc2)(c2ccccc2)c2ccccc2)CC1 ; [I-]]

### Steps 1, 2, 3, and 4 are the Same as Steps 1, 2, 3, and 4 for Example 5

### Step 5. N-(((1S,4S)-4-Hydroxy-1-phenylcyclohexyl)methyl)-2-methoxybenzamide

Synthesized from 2-methoxy-N-((4-oxo-1-phenylcyclohexyl)methyl)benzamide (2.91 g, 8.63 mmol, 1 equiv) and NaBH₄ (0.66 g, 17.27 mmol, 2 equiv) via general procedure H. Column chromatography, DCM/diethyl ether=3/1 (v/v). Yield: 39% (1.14 g); white solid. ¹H NMR (400 MHz, DMSO-d₆): δ_{H} 1.48-1.62 (4H, m, Hₐ-3,5, Hₑ-3,5), 1.68-1.80 (2H, m, Hₐ-2,6), 1.94-2.04 (2H, m, Hₑ-2,6), 3.47-3.56 (1H, m, H-4), 3.61 (5H, d, J=3.1 Hz, OCH₃, H-7′, H-7″), 4.51 (1H, d, J=4.0 Hz, OH), 6.98-7.04 (1H, m, H-13), 7.06 (1H, d, J=8.0 Hz, H-11), 7.27 (1H, t, J=7.1 Hz, H-19), 7.38-7.46 (5H, m, H-12, H-17, H-18, H-20, H-21), 7.55 (1H, brt, J=5.5 Hz, NHCO), 7.86 (1H, dd, J₁=7.7 Hz,J₂=1.8 Hz, H-14). ¹³C NMR (101 MHz, DMSO-d₆): δ_{C} 29.64 (C-2,6), 30.25 (C-3,5), 40.96 (C-1), 47.43 (C-7), 55.68 (C-15), 66.54 (C-4), 112.08 (C-11), 120.69 (C-13), 121.27 (C-9), 126.02 (C-19), 126.41 (C-18,20), 128.42 (C-17,21), 131.02 (C-14), 132.62 (C-12), 145.47 (C-16), 157.04 (C-10), 164.06 (C-8). HRMS (ESI+): m/z calcd for [M+H]⁺ 340.1907; found 340.1906. HPLC purity, 97.6% at 254 nm (t_{R}=4.46 min).

### Steps 6, 7, and 8 are the Same as Steps 9, 10, and 11 for Example 1

### Step 9. (3-(((((1S,4S)-4-((2-methoxybenzamido)methyl)-4-phenylcyclohexyl)oxy)carbonyl)amino)propyl)triphenylphosphonium iodide

Synthesized from N-(((1S,4S)-4-hydroxy-1-phenylcyclohexyl)methyl)-2-methoxybenzamide (100 mg, 0.29 mmol, 1.0 equiv), 4-nitrophenyl chloroformate (119 mg, 0.58 mmol, 2 equiv), Et₃N (0.12 mL, 0.87 mmol, 3 equiv) and (3-aminopropyl)triphenylphosphonium iodide (158 mg, 0.35 mmol, 1.2 equiv) according to general procedure I. Column chromatography, DCM/MeOH=100/1 (v/v). Two confomers in a 13:87 ratio. Yield: 22% (52 mg); white solid. ¹H NMR (400 MHz, DMSO) for both confomers: δ 7.95-7.88 (3H, m, H-29, H-35, H-41), 7.88-7.73 (13H, m, H-14, H-27, H-28, H-30, H-31, H-33, H-34, H-36, H-37, H-39, H-40, H-42, H-43), 7.55 (1H, t, J=5.6 Hz, CH₂NHCO), 7.50-7.40 (5H, m, H-12, H-17, H-18, H-20, H-21), 7.34-7.23 (2H, m, H-19, OCONHCH₂), 7.07 (1H, d, J=8.3 Hz, H-11), 7.02 (1H, t, J=7.5 Hz, H-13), 4.62-4.46 (1H, m, H-4), 3.60 (3H, s, OCH₃), 3.67-3.49 (4H, m, H-25′, H-25″, H-7′, H-7″), 3.16 (2H, dd, J₁=11.4 Hz, J₂=5.7 Hz, H-23′. H-23″), 2.04-1.78 (4H, m, Hₑ-2,6, Hₐ-2,6), 1.78-1.42 (6H, m, Hₑ-3,5, Hₐ-3,5, H-24′, H-24″); ¹³C NMR (101 MHz, DMSO) for both confomers: δ 164.18 (C-8), 157.05 (C-10), 155.77 (C-22), 144.46 (C-16), 134.98 (d, J=2.6 Hz, C-29, C-35, C-41), 133.57 (d, J=10.2 Hz, C-28, C-30, C-34, C-36, C-40, C-42), 132.72 (C-12), 130.98 (C-14), 130.29 (d, J=12.5 Hz, C-27, C-31, C-33, C-37, C-39, C-43), 128.56 (C-18, C-20), 126.51 (C-17, C-21), 126.22 (C-19), 121.17 (C-9), 120.70 (C-13), 118.33 (d, J=86.0 Hz, C-26, C-32, C-38), 112.12 (C-11), 70.24 (C-4), 55.72 (C-15), 47.99 (C-7), 41.06 (C-1), 40.33 (C-23), 29.37 (C-2,6), 26.89 (C-3,5), 22.43 (C-24), 18.24 (d, J=51.6 Hz, C-25); HRMS (ESI+): m/z calcd for [M+H]⁺ 685.3190; found 685.3182. HPLC purity, 96.55% at 254 nm (t_{R}=4.967 min).

### Example 7

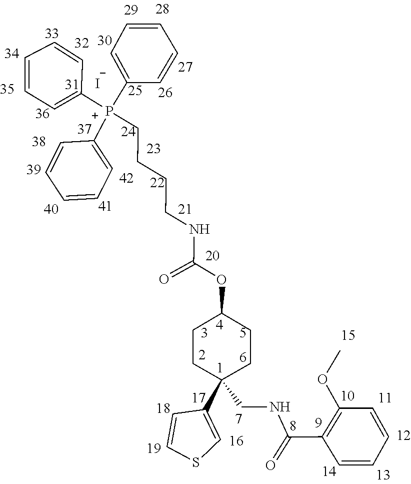 [SMILES: COc1ccccc1C(=O)NC[C@]1(c2ccsc2)CC[C@@H](OC(=O)NCCCC[P+](c2ccccc2)(c2ccccc2)c2ccccc2)CC1 ; [I-]]

### Steps 1, 2, 3, 4, 5, 6, 7, and 8 are the Same as Steps 1, 2, 3, 4, 5, 6, 7, and 8 for Example 1

### Step 9. (4-Iodobutyl)triphenylphosphonium iodide

Synthesized from triphenylphosphine (3.02 g, 11.5 mmol, 1.0 equiv) and 1,4-diiodobutane (3.03 mL, 23 mmol, 2.0 equiv) via general procedure J. The product was used without further purification. Yield: 99.4% (6.54 g); white crystals. ¹H NMR (400 MHz, CDCl₃): δ 1.83 (dt, J, =15.4 Hz, J₂=7.8 Hz, 2H), 2.16-2.29 (m, 2H), 3.33 (t, J=6.3 Hz, 2H), 3.70-3.84 (m, 2H), 7.67-7.78 (m, 6H), 7.79-7.92 (m, 9H). HRMS (ESI+): m/z calcd for [M+H]⁺ 445.05766; found 445.05595.

### Step 10. (4-Azidobutyl)triphenylphosphonium iodide

Synthesized from (4-iodobutyl)triphenylphosphonium iodide (3.0 g, 5.24 mmol, 1.0 equiv) and sodium azide (0.68 g, 10.48 mmol, 2.0 equiv) via general procedure K. The product was used without further purification. Yield: 99.5% (2.54 g). ¹H NMR (400 MHz, CDCl₃): δ 1.76 (tt, J₁=15.8 Hz, J₂=8.1 Hz, 2H), 1.97-2.11 (m, 2H), 3.45 (t, J=6.2 Hz, 2H), 3.74-3.90 (m, 2H), 7.67-7.77 (m, 6H), 7.77-7.91 (m, 9H).

### Step 11. (4-Aminobutyl)triphenylphosphonium iodide

Synthesized from (4-azidobutyl)triphenylphosphonium iodide (2.42 g, 5.0 mmol, 1.0 equiv) and Pd/C (250 mg) via general procedure L. The product was used without further purification. Yield: 98.0% (2.26 g). ¹H NMR (400 MHz, CDCl₃): δ 1.43 (s, 2H), 1.66-1.81 (m, 2H), 1.86 (dt, J₁=13.9 Hz, J₂=6.9 Hz, 2H), 2.77 (t, J=6.6 Hz, 2H), 3.70-3.85 (m, 2H), 7.65-7.76 (m, 6H), 7.77-7.90 (m, 9H). HRMS (ESI+): m/z calcd for [M+H]⁺ 334.17191; found 334.17069.

### Step 12. (4-(((((1R,4R)-4-((2-Methoxybenzamido)methyl)-4-(thiophen-3-yl)cyclohexyl)oxy)carbonyl)amino)butyl)triphenylphosphonium iodide

Synthesized from N-(((1R,4R)-4-hydroxy-1-(thiophen-3-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (4-aminobutyl)triphenylphosphonium iodide (161 mg, 0.35 mmol, 1.2 equiv) according to general procedure I. Column chromatography, DCM/MeOH=100/1 (v/v). Two confomers in a 7:93 ratio. Yield: 25% (60 mg); white solid. ¹H NMR (400 MHz, DMSO) for both confomers: δ 7.96-7.85 (3H, m, H-28, H-34, H-40), 7.83 (1H, dd, J₁=7.7 Hz, J₂=1.8 Hz, H-14), 7.81-7.67 (13H, m, H-26, H-27, H-29, H-30, H-32, H-33, H-35, H-36, H-38, H-39, H-41, H-42, CH₂NHCO), 7.62 (1H, dd, J₁=4.9 Hz, J₂=2.9 Hz, H-19), 7.47 (1H, ddd, J₁=8.4 Hz, J₂=7.3 Hz, J₃=1.9 Hz, H-12), 7.40 (1H, d, J=1.5 Hz, H-16), 7.20 (1H, dd, J₁=5.0 Hz, J₂=0.9 Hz, H-18), 7.12 (1H, d, J=7.9 Hz, H-11), 7.08-7.00 (1H, m, H-13), 6.97 (1H, t, J=5.8 Hz, OCONHCH₂), 4.60-4.40 (1H, m, H-4), 3.77 (3H, s, OCH₃), 3.64-3.49 (2H, m, H-24′, H-24″), 3.44 (2H, d, J=5.7 Hz, H-7′, H-7″), 2.97 (2H, dd, J₁=11.6 Hz, J₂=5.8 Hz, H-21′, H-21″), 2.23-2.04 (2H, m, Hₑ-2,6), 1.82-1.69 (2H, m, He-3,5), 1.68-1.38 (6H, m, Hₐ-2,6, H-22′, H-22″, H-23′, H-23″), 1.36-1.17 (2H, m, Hₐ-3,5);. ¹³C NMR (101 MHz, DMSO) for both confomers: δ 164.40 (C-8), 157.03 (C-10), 155.79 (C-20), 145.61 (C-17), 134.90 (d, J=2.9 Hz, C-28, C-34, C-40), 133.57 (d, J=10.1 Hz, C-27, C-29, C-33, C-35, C-39, C-41), 132.53 (C-12), 130.84 (C-14), 130.23 (d, J=12.4 Hz, C-26, C-30, C-32, C-36, C-38, C-42), 126.68 (C-18), 126.43 (C-19), 121.81 (C-16), 121.71 (C-9), 120.67 (C-13), 118.46 (d, J=85.7 Hz, C-25, C-31, C-37), 112.09 (C-11), 71.35 (C-4), 55.87 (C-15), 49.66 (C-7), 40.65 (C-1), 38.89 (C-21), 31.01 (C-2,6), 30.22 (d, J=16.6 Hz, C-22), 27.39 (C-3,5), 19.90 (d, J=49.7 Hz, C-24), 19.16 (d, J=4.8 Hz, C-23); HRMS (ESI+): m/z calcd for [M+H]⁺ 705.2910; found 705.2900. HPLC purity, 99.86% at 254 nm (t_{R}=4.797 min).

### Example 8

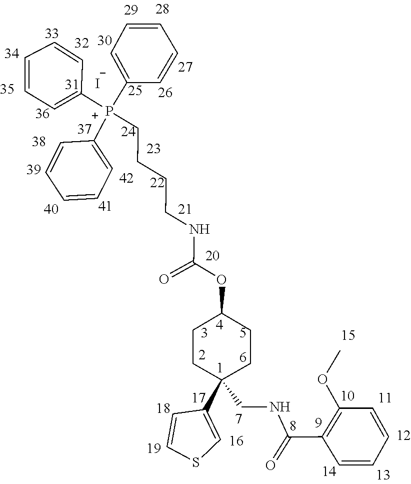 [SMILES: COc1ccccc1C(=O)NC[C@]1(c2ccsc2)CC[C@@H](OC(=O)NCCCC[P+](c2ccccc2)(c2ccccc2)c2ccccc2)CC1 ; [I-]]

### Steps 1, 2, 3, 4, 5, 6, 7, and 8 are the Same as Steps 1, 2, 3, 4, 5, 6, 7, and 8 for Example 2

### Steps 9, 10, and 11 are the Same as Steps 9, 10, and 11 for Example 7

### Step 12. (4-(((((1S,4S)-4-((2-Methoxybenzamido)methyl)-4-(thiophen-3-yl)cyclohexyl)oxy)carbonyl)amino)butyl)triphenylphosphonium iodide

Synthesized from N-(((1S,4S)-4-hydroxy-1-(thiophen-3-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (4-aminobutyl)triphenylphosphonium iodide (161 mg, 0.35 mmol, 1.2 equiv) according to general procedure I. Column chromatography, DCM/MeOH=100/1 (v/v). Two confomers in a 11:89 ratio. Yield: 22% (52 mg); white solid. ¹H NMR (400 MHz, DMSO) for both confomers: δ 7.95-7.86 (4H, m, H-14, H-28, H-34, H-40), 7.85-7.72 (12H, m, H-26, H-27, H-29, H-30, H-32, H-33, H-35, H-36, H-38, H-39, H-41, H-42), 7.65 (1H, t, J=5.7 Hz, CH₂NHCO), 7.62 (1H, dd, J₁=5.0 Hz, J₂=2.9 Hz, H-19), 7.52-7.43 (1H, m, H-12), 7.39 (1H, dd, J₁=2.7 Hz, J₂=1.1 Hz, H-16), 7.26-7.19 (1H, m, H-18), 7.16-7.08 (2H, m, H-11, OCONHCH₂), 7.04 (1H, t, J=7.5 Hz, H-13), 4.59-4.44 (1H, m, H-4), 3.72 (3H, s, OCH₃), 3.65-3.51 (4H, m, H-7′, H-7″, H-24′, H-24″), 3.02 (2H, q, J=6.0 Hz, H-21′, H-21″), 1.94-1.72 (4H, m, Hₑ-2,6, Hₐ-2,6), 1.70-1.43 (8H, m, Hₑ-3,5, Hₐ-3,5, H-22′, H-22″, H-23′, H-23″); ¹³C NMR (101 MHz, DMSO) for both confomers: δ 164.17 (C-8), 157.15 (C-10), 155.87 (C-20), 146.85 (C-17), 134.92 (d, J=2.6 Hz, C-28, C-34, C-40), 133.58 (d, J=10.2 Hz, C-27, C-29, C-33, C-35, C-39, C-41), 132.78 (C-12), 131.06 (C-14), 130.25 (d, J=12.4 Hz, C-26, C-30, C-32, C-36, C-38, C-42), 126.54 (C-18), 126.40 (C-19), 121.13 (C-9, C-16), 120.74 (C-13), 118.48 (d, J=85.8 Hz, C-25, C-31, C-37), 112.18 (C-11), 70.10 (C-4), 55.85 (C-15), 47.27 (C-7), 40.31 (C-1), 38.89 (C-21), 30.35 (C-22), 30.24 (C-2,6), 26.87 (C-3,5), 19.95 (d, J=50.8 Hz, C-24), 19.21 (d, J=3.2 Hz, C-23); HRMS (ESI+): m/z calcd for [M+H]⁺ 705.2910; found 705.2887. HPLC purity, 96.20% at 254 nm (t_{R}=4.963 min).

### Example 9

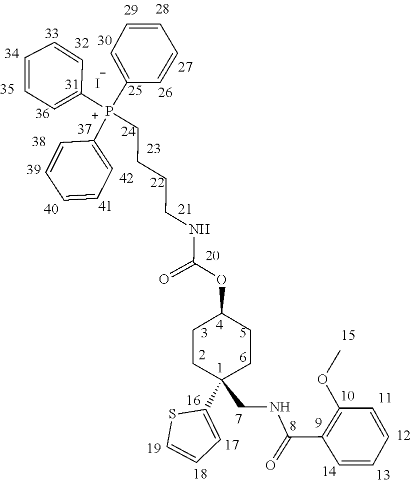 [SMILES: COc1ccccc1C(=O)NC[C@]1(c2cccs2)CC[C@H](OC(=O)NCCCC[P+](c2ccccc2)(c2ccccc2)c2ccccc2)CC1 ; [I-]]

### Steps 1, 2, 3, 4, 5, 6, 7, and 8 are the Same as Steps 1, 2, 3, 4, 5, 6, 7, and 8 for Example 3

### Steps 9, 10, and 11 are the Same as Steps 9, 10, and 11 for Example 7

### Step 12. (4-(((((1R,4R)-4-((2-Methoxybenzamido)methyl)-4-(thiophen-2-yl)cyclohexyl)oxy)carbonyl)amino)butyl)triphenylphosphonium iodide

Synthesized from N-(((1R,4R)-4-hydroxy-1-(thiophen-2-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (4-aminobutyl)triphenylphosphonium iodide (161 mg, 0.35 mmol, 1.2 equiv) according to general procedure I. Column chromatography, DCM/MeOH=100/1 (v/v). Two confomers in a 11:89 ratio. Yield: 21% (51 mg); white solid. ¹H NMR (400 MHz, DMSO) for both confomers: δ 7.94-7.86 (4H, m, H-14, H-28, H-34, H-40), 7.85-7.70 (13H, m, H-26, H-27, H-29, H-30, H-32, H-33, H-35, H-36, H-38, H-39, H-41, H-42, CH₂NHCO, H-14), 7.51 (1H, dd, J₁=5.1, J₂=0.8 Hz, H-19), 7.51-7.44 (1H, m, H-12), 7.14 (1H, t, J=6.0 Hz, OCONHCH₂), 7.17-7.08 (1H, m, H-11), 7.10 (1H, dd, J₁=5.1 Hz, J₂=3.6 Hz, H-18), 7.08-6.96 (2H, m, H-17, H-13), 4.67-4.43 (1H, m, H-4), 3.73 (3H, s, OCH₃), 3.67-3.50 (4H, m, H-7′, H-7″, H-24′, H-24″), 3.02 (2H, dd, J₁=11.5 Hz, J₂=5.7 Hz, H-21′, H-21″), 1.99-1.75 (4H, m, Hₑ-2,6, Hₐ-2,6), 1.73-1.44 (8H, m, Hₑ-3,5, Hₐ- 3,5, H-22′, H-22″, H-23′, H-23″); ¹³C NMR (101 MHz, DMSO) for both confomers: δ 164.31 (C-8), 157.16 (C-10), 155.84 (C-20), 150.21 (C-16), 134.91 (d, J=2.8 Hz, C-28, C-34, C-40), 133.58 (d, J=10.1 Hz, C-27, C-29, C-33, C-35, C-39, C-41), 132.81 (C-12), 131.02 (C-14), 130.25 (d, J=12.4 Hz, C-26, C-30, C-32, C-36, C-38, C-42), 127.03 (C-18), 124.41 (C-16), 124.32 (C-19), 121.22 (C-9), 120.74 (C-13), 118.48 (d, J=85.7 Hz, C-25, C-31, C-37), 112.22 (C-11), 69.55 (C-4), 55.84 (C-15), 49.02 (C-7), 41.02 (C-1), 38.89 (C-21), 31.10 (C-2,6), 30.71 (C-22), 26.73 (C-3,5), 19.94 (d, J=50.3 Hz, C-24), 19.21 (d, J=4.3 Hz, C-23); HRMS (ESI+): m/z calcd for [M+H]⁺ 705.2910; found 705.2888. HPLC purity, 97.69% at 254 nm (t_{R}=4.997 min).

### Example 10

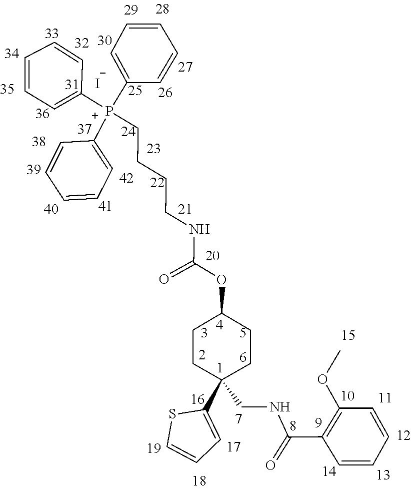 [SMILES: COc1ccccc1C(=O)NC[C@]1(c2cccs2)CC[C@@H](OC(=O)NCCCC[P+](c2ccccc2)(c2ccccc2)c2ccccc2)CC1 ; [I-]]

### Steps 1, 2, 3, 4, 5, 6, 7, and 8 are the Same as 1, 2, 3, 4, 5, 6, 7, and 8 for Example 4

### Steps 9, 10, and 11 are the Same as Steps 9, 10, and 11 for Example 7

### Step 12. (4-(((((1S,4S)-4-((2-Methoxybenzamido)methyl)-4-(thiophen-2-yl)cyclohexyl)oxy)carbonyl)amino)butyl)triphenylphosphonium iodide

Synthesized from N-(((1S,4S)-4-hydroxy-1-(thiophen-2-yl)cyclohexyl)methyl)-2-methoxybenzamide (100 mg, 0.29 mmol, 1.0 equiv), 4-nitrophenyl chloroformate (117 mg, 0.58 mmol, 2 equiv), Et₃N (0.12 mL, 0.87 mmol, 3 equiv) and (4-aminobutyl)triphenylphosphonium iodide (161 mg, 0.35 mmol, 1.2 equiv) according to general procedure I. Column chromatography, DCM/MeOH=100/1 (v/v). Two confomers in a 7:93 ratio. Yield: 23% (55 mg); white solid. ¹H NMR (400 MHz, DMSO) for both confomers: δ 7.94-7.85 (4H, m, H-28, H-34, H-40, CH₂NHCO), 7.82 (1H, dd, J₁=7.7 Hz, J₂=1.7 Hz, H-14), 7.80-7.70 (12H, m, H-26, H-27, H-29, H-30, H-32, H-33, H-35, H-36, H-38, H-39, H-41, H-42), 7.52 (1H, d, J=4.8 Hz, H-19), 7.48 (1H, ddd, J₁=9.1 Hz, J₂=7.4 Hz, J₃=1.8 Hz, H-12), 7.13 (1H, d, J=8.3 Hz, H-11), 7.10 (1H, dd, J₂=4.9 Hz, 3.7 Hz, H-18), 7.07-7.01 (2H, m, H-13, H-17), 6.99 (1H, t, J=5.8 Hz, OCONHCH₂), 4.62-4.39 (1H, m, H-4), 3.79 (3H, s, OCH₃), 3.64-3.50 (2H, m, H-24′, H-24″), 3.46 (2H, d, J=5.9 Hz, H-7′, H-7″), 2.98 (2H, dd, J₁=11.1 Hz, J₂=5.3 Hz, H-21′, H-21″), 2.18-2.00 (2H, m, Hₑ-2,6), 1.85-1.66 (4H, m, Hₑ-3,5, Hₐ-2,6), 1.64-1.42 (4H, m, H-22′, H-22″, H-23′, H-23″), 1.42-1.24 (2H, m, Hₐ-3,5); ¹³C NMR (101 MHz, DMSO) for both confomers: δ 164.58 (C-8), 157.01 (C-10), 155.76 (C-20), 149.38 (C-16), 134.89 (d, J=2.4 Hz, C-28, C-34, C-40), 133.56 (d, J=10.1 Hz, C-27, C-29, C-33, C-35, C-39, C-41), 132.52 (C-12), 130.75 (C-14), 130.22 (d, J=12.4 Hz, C-26, C-30, C-32, C-36, C-38, C-42), 127.12 (C-18), 124.68 (C-16), 124.57 (C-19), 121.91 (C-9), 120.65 (C-13), 118.46 (d, J=85.7 Hz, C-25, C-31, C-37), 112.11 (C-11), 71.07 (C-4), 55.85 (C-15), 50.94 (C-7), 41.60 (C-1), 38.89 (C-21), 31.97 (C-3,5), 30.21 (d, J=16.9 Hz, C-22), 27.34 (C-3,5), 19.91 (d, J=50.0 Hz, C-24), 19.15 (d, J=3.6 Hz, C-23); HRMS (ESI+): m/z calcd for [M+H]⁺ 705.2897; found 705.2901. HPLC purity, 96.67% at 254 nm (t_{R}=4.80 min).

### Example 11

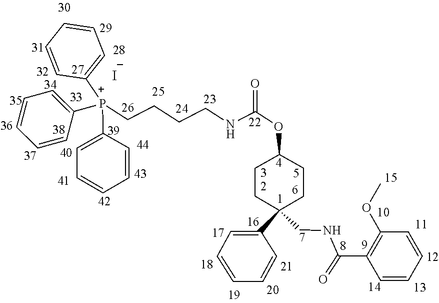 [SMILES: COc1ccccc1C(=O)NC[C@]1(c2ccccc2)CC[C@@H](OC(=O)NCCCC[P+](c2ccccc2)(c2ccccc2)c2ccccc2)CC1 ; [I-]]

### Steps 1, 2, 3, 4, and 5 are the Same as Steps 1, 2, 3, 4, and 5 for Example 5

### Steps 6, 7, and 8 are the Same as Steps 9, 10, and 11 for Example 7

### Step 9. (4-(((((1R,4R)-4-((2-Methoxybenzamido)methyl)-4-phenylcyclohexyl)oxy)carbonyl)amino)butyl)triphenylphosphonium iodide

Synthesized from N-(((1R,4R)-4-hydroxy-1-phenylcyclohexyl)methyl)-2-methoxybenzamide (100 mg, 0.29 mmol, 1.0 equiv), 4-nitrophenyl chloroformate (119 mg, 0.58 mmol, 2 equiv), Et₃N (0.12 mL, 0.87 mmol, 3 equiv) and (4-aminobutyl)triphenylphosphonium iodide (161 mg, 0.35 mmol, 1.2 equiv) according to general procedure I. Column chromatography, DCM/MeOH=100/1 (v/v). Two confomers in a 7:93 ratio. Yield: 21% (50 mg); white solid. ¹H NMR (400 MHz, DMSO) for both confomers: δ 7.94-7.84 (3H, m, H-30, H-36, H-42), 7.80 (1H, dd, J₁=7.7 Hz, J₂=1.8 Hz, H-14), 7.82-7.72 (12H, m, H-28, H-29, H-31, H-32, H-34, H-35, H-37, H-38, H-40, H-41, H-43, H-44), 7.69 (1H, t, J=5.8 Hz, CH₂NHCO), 7.50-7.40 (5H, m, H-12, H-17, H-18, H-20, H-21), 7.36-7.23 (1H, m, H-19), 7.10 (1H, d, J=8.0 Hz, H-11), 7.06-6.99 (1H, m, H-13), 6.94 (1H, t, J=5.7 Hz, OCONHCH₂), 4.63-4.42 (1H, m, H-4), 3.70 (3H, s, OCH₃), 3.63-3.49 (2H, m, H-26′, H-26″), 3.46 (2H, d, J=5.7 Hz, H-7′, H-7″), 2.96 (2H, dd, J₁=11.6 Hz, J₂=5.7 Hz, H-23′, H-23″), 2.31-2.12 (2H, m, Hₑ-2,6), 1.87-1.69 (2H, m, Hₑ-3,5), 1.65 (2H, t, J=12.7 Hz, Hₐ-2,6), 1.60-1.36 (4H, m, H-24′, H-24″, H-25′, H-25″), 1.21 (2H, dd, J₁=20.6 Hz, J₂=9.6 Hz, Hₐ-3,5); ¹³C NMR (101 MHz, DMSO) for both confomers: δ 164.43 (C-8), 156.95 (C-10), 155.77 (C-22), 143.36 (C-16), 134.88 (d, J=2.8 Hz, C-30, C-36, C-42), 133.55 (d, J=10.1 Hz, C-29, C-31, C-35, C-37, C-41, C-43), 132.49 (C-12), 130.78 (C-14), 130.21 (d, J=12.4 Hz, C-28, C-32, C-34, C-38, C-40, C-44), 128.62 (C-18, C-20), 126.89 (C-17, C-21), 126.12 (C-19), 121.85 (C-9), 120.65 (C-13), 118.45 (d, J=85.7 Hz, C-27, C-33, C-39), 112.06 (C-11), 71.44 (C-4), 55.78 (C-15), 50.19 (C-7), 41.80 (C-1), 39.98 (C-23), 30.28 (C-24), 30.12 (C-2,6), 27.36 (C-3,5), 19.90 (d, J=49.0 Hz, C-26), 19.13 (d, J=2.9 Hz, C-25). HRMS (ESI+): m/z calcd for [M+H]⁺ 699.3346; found 699.3337. HPLC purity, 96.69% at 254 nm (t_{R}=4.843 min).

### Example 12

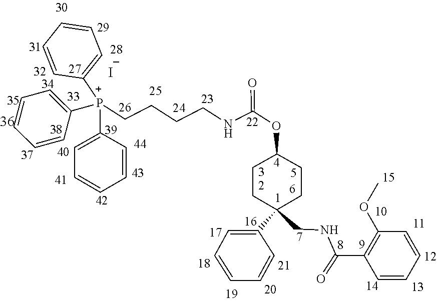 [SMILES: COc1ccccc1C(=O)NC[C@]1(c2ccccc2)CC[C@H](OC(=O)NCCCC[P+](c2ccccc2)(c2ccccc2)c2ccccc2)CC1 ; [I-]]

### Steps 1, 2, 3, 4, and 5 are the Same as Steps 1, 2, 3, 4, and 5 for Example 6

### Steps 6, 7, and 8 are the Same as Steps 9, 10, and 11 for Example 7

### Step 9. (4-(((((1S,4S)-4-((2-Methoxybenzamido)methyl)-4-phenylcyclohexyl)oxy)carbonyl)amino)butyl)triphenylphosphonium iodide

Synthesized from N-(((1S,4S)-4-hydroxy-1-phenylcyclohexyl)methyl)-2-methoxybenzamide (100 mg, 0.29 mmol, 1.0 equiv), 4-nitrophenyl chloroformate (119 mg, 0.58 mmol, 2 equiv), Et₃N (0.12 mL, 0.87 mmol, 3 equiv) and (4-aminobutyl)triphenylphosphonium iodide (161 mg, 0.35 mmol, 1.2 equiv) according to general procedure I. Column chromatography, DCM/MeOH=100/1 (v/v). Two confomers in a 1:9 ratio. Yield: 21% (50 mg); white solid. ¹H NMR (400 MHz, DMSO) for both confomers: δ 7.94-7.86 (4H, m, H-14, H-30, H-36, H-42), 7.84-7.71 (12H, m, H-28, H-29, H-31, H-32, H-34, H-35, H-37, H-38, H-40, H-41, H-43, H-44), 7.55 (1H, t, J=5.6 Hz, CH₂NHCO), 7.51-7.40 (51H, m, H-12, H-17, H-18, H-20, H-21), 7.34-7.26 (1H, m, H-19), 7.13 (1H, t, J=5.7 Hz, OCONHCH₂), 7.07 (1H, d, J=8.2 Hz, H-11), 7.02 (1H, t, J=7.5 Hz, H-13), 4.69-4.35 (1H, m, H-4), 3.60 (31H, s, OCH₃), 3.72-3.47 (4H, m, H-7′, H-7″, H-26′, H-26″), 3.02 (21H, q, J=6.1 Hz, H-23′, H-23″), 2.00- 1.74 (41H, m, Ha-2,6, Hₑ-2,6), 1.73-1.31 (8H, m, H-24′, H-24″, H-25′, H-25″, Hₐ-3,5, Hₑ-3,5); ¹³C NMR (101 MHz, CDCl₃) for both confomers: δ 165.15 (C-8), 157.42 (C-10), 156.68 (C-22), 144.29 (C-16), 135.16 (d, J=2.9 Hz, C-30, C-36, C-42), 133.71 (d, J=9.9 Hz, C-29, C-31, C-35, C-37, C-41, C-43), 132.77 (C-12), 132.14 (C-14), 130.56 (d, J=12.6 Hz, C-28, C-32, C-34, C-38, C-40, C-44), 128.68 (C-18, C-20), 126.68 (C-17, C-21), 126.38 (C-19), 121.03 (C-13), 120.80 (C-9), 117.97 (d, J=86.1 Hz, C-27, C-33, C-39), 111.26 (C-11), 71.02 (C-4), 55.56 (C-15), 49.31 (C-7), 41.53 (C-1), 39.14 (C-23), 30.44 (C-24), 29.66 (C-2,6), 27.02 (C-3,5), 22.40 (d, J=51.2 Hz, C-26), 19.36 (d, J=2.4 Hz, C-25); HRMS (ESI+): m/z calcd for [M+H]⁺ 699.3346; found 699.3339. HPLC purity, 95.44% at 254 nm (t_{R}=5.073 min).

### Biological Data

To test the activity of the compounds on biological systems, we chose the pancreatic cancer cell line Colo 357, which expresses Kv1.3 and is sensitive to cell-permeant Kv1.3 inhibitors (Zaccagnino et al., Oncotarget 8, 38276-38293, 2017). Growth, cell viability and apoptosis were all determined by high content imaging in a Incucyte (Sartorius) live cell imaging device. Images were taken every hour for several days after addition of the compounds dissolved in DMSO (time 0) and the corresponding indicators, CytotoxGreen or CytotoxRed for cytotoxicity and the green Caspase 3/7 activity reporter for apoptosis.

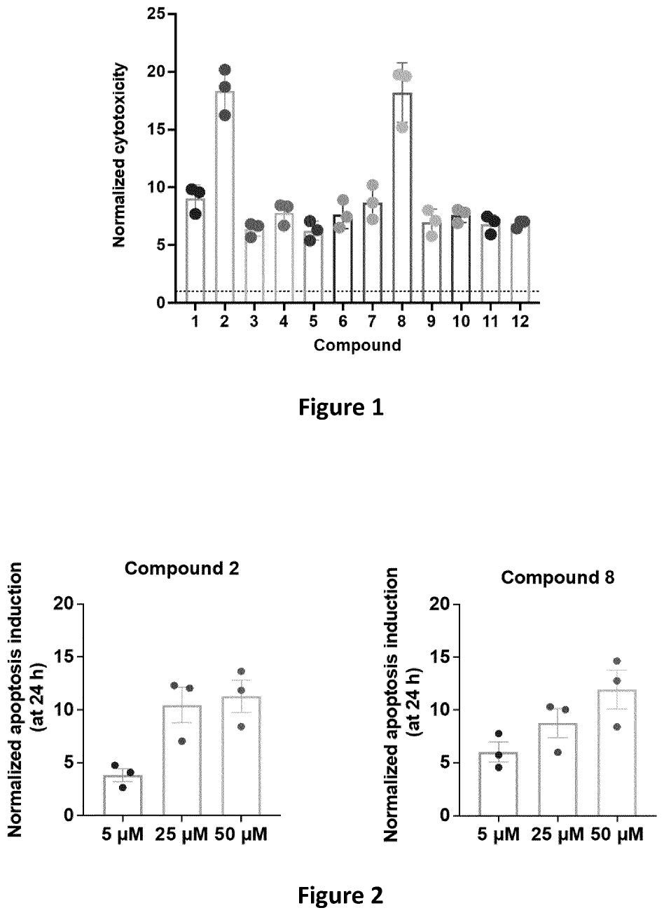

The first screening for cytotoxicity was performed on conventional 2D cultures (FIG. 1). All tested compounds induced significant cell death after 24 hours of treatment. The most intense effect was observed in the presence of example compounds 2 and 8, and these two were selected for further characterization.

The toxic effect of both compounds was induced through apoptosis, as expected from mitoKv1.3 inhibitors Urbani, A., et al., Front Cell Dev Biol 8, 620081, 2021). FIG. 2 represents apoptosis induction after 24 h incubation in the presence of compound 2 and 8 at the indicated concentrations. Both compounds induced an strong increase in apoptotic cells already at 5 μM, and the intensity of the effect increased in a dose-dependent manner.

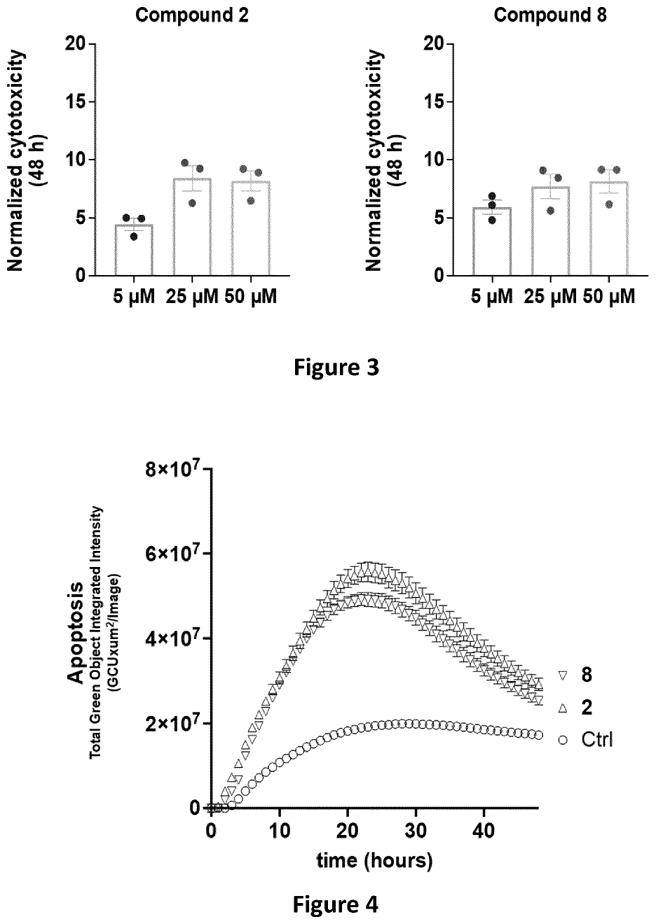

To obtain information on the effect of the compounds in a model more similar to the situation in vivo, the effects of 2 and 8 were also studied in tumor spheroids. Spheroids were formed in round bottom ultra-low attachment 96-well plates (Corning) in 2% Matrigel in culture medium. The treatments were added once the spheroids were formed. Cytotoxicity was determined as in the conventional 2D cultures but after 48 h treatment instead of 24 h, because the effects needed longer time to develop. As shown in FIG. 3, the compounds induced significant levels of cytotoxicity with concentrations as low as 5 μM also in 3-D cultures.

Cell death in 3D culture was also attributable to induction of apoptosis. FIG. 4 presents the time course of apoptosis induction in the presence of 5 μM of the compounds. The fluorescence of the caspase 3/7 reporter was detectable soon after addition of the treatment and was markedly more intense than the values measured in the control treatment (DMSO).

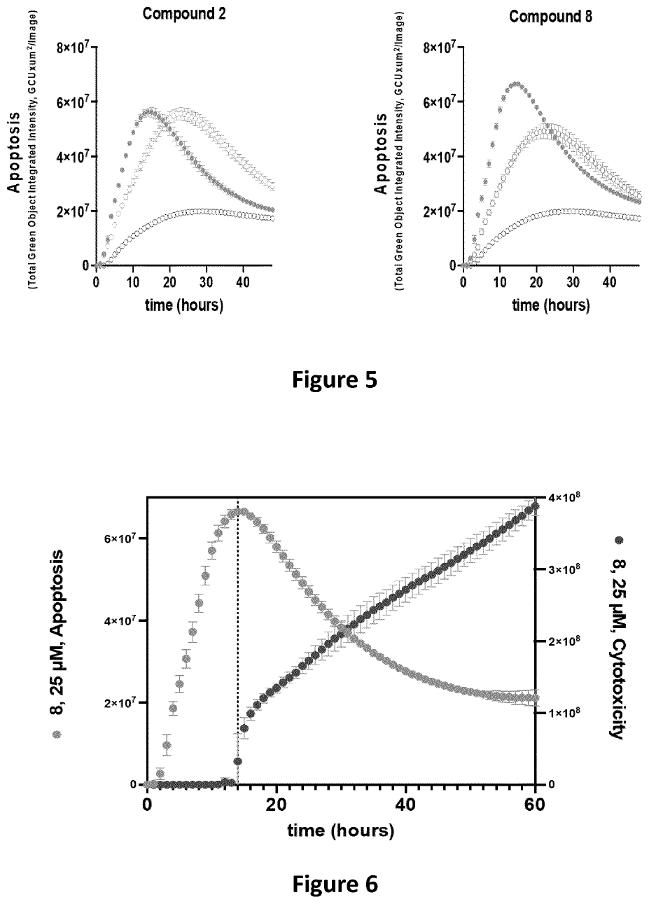

The effect of the compounds was concentration-dependent. Increasing the concentration of the inhibitor to 25 μM had little effect on the absolute magnitude of apoptosis induction, but clearly accelerated the effect (FIG. 5).

The reason for the decline in fluorescence after ˜12 or ˜24 hours is likely the reduction in the number of viable cells in the spheroid. Thus, comparing the time course of the development of apoptosis and of cytotoxicity (FIG. 6, for compound 8 at 25 μM), it becomes visible that the initiation of the decline in apoptosis signal corresponds to the start of the increase in the number of dead cells.

1. A compound of formula (I): 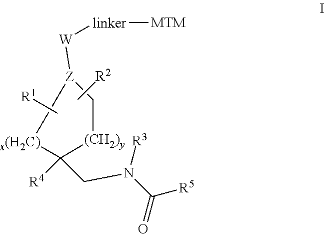
wherein:
“MTM” is a mitochondria targeting moiety;
x and y are independently 0, 1, or 2;
Z is CH or N;
R¹ and R² are independently selected from the group consisting of hydrogen, halo, wherein halo is fluoro, chloro, bromo, or iodo, hydroxy, HO(C₁-C₆)-alkyloxy, (C₁-C₄)-perfluoroalkyl, O(CO)CCl₃, (C₁-C₆)-alkyl-S(O)ₙ—, phenyl-(CH₂)ᵣ—S(O)ₙ—, cyano, nitro, COOH, CO(C₁-C₆)-alkyl, COO(C₁-C₆)-alkyl, CONR⁶R⁷, NR⁶R⁷, O(CO)NR⁶R⁷, azido, NR⁶(CO)NR⁶R⁷, (C₁-C₁₀)-alkyl, (C₂-C₁₀)-alkenyl, (C₂-C₁₀)-alkynyl, O[(C═O)Oᵣ]ₛ(C₁-C₆)-alkyl, O[(C═O)Oᵣ]ₛ(C₂-C₆)-alkenyl, O[(C═O)Oᵣ]ₛaryl, O[(C═O)Oᵣ]ₛheteroaryl, O(CH₂)ₙheteroaryl, aryl, O(CH₂)ₙaryl, oxo, ═CH—(C₁-C₆)-alkyl, ═CH—(C₂-C₆)-alkenyl, ═CH-aryl, and ═CH₂, with r and s at each occurrence being independently from each other 0 or 1, and n at each occurrence being 0, 1, 2 or 3;
R³ is hydrogen, [(C═O)Oᵣ]ₛaryl or [(C═O)Oᵣ]ₛ(C₁-C₆)-alkyl, with r and s at each occurrence being independently from each other 0 or 1;
R⁴ and R⁵ are independently selected from the group consisting of substituted or unsubstituted aryl, and substituted or unsubstituted five or six membered heterocyclyl containing from 1 to 3 heteroatoms selected from the group consisting of O, N and S;
wherein the aryl, if substituted, is substituted with one or more substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)₁—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3;
wherein the heterocyclyl, if substituted, is substituted with one or more substituents selected from the group consisting of halo, wherein halo is fluoro, chloro, bromo, or iodo, hydroxy, (C₁-C₆)-alkyl, (C₁-C₄)-perfluoroalkyl, (C₂-C₆)-alkenyl, (C₂-C₆)-alkynyl, (C₁-C₆)-alkyloxy, (C₁-C₆)-alkyl-S(O)ₙ—, O(C₀-C₆)-alkyl-S(O)ₙ—, phenyl, phenoxy, cyano, nitro, COOH, CO(C₁-C₆)-alkyl, COO(C₁-C₆)-alkyl, CONR⁶R⁷, NR⁶R⁷, methylenedioxyl, OCF₃, and fused benzo or pyridyl group, with n at each occurrence being 0, 1, 2 or 3;
R⁶ and R⁷ are independently selected from the group consisting of hydrogen, [(C═O)Oᵣ]ₛaryl, [(C═O)Oᵣ]ₛ(C₂-C₈)-alkenyl, [(C═O)Oᵣ]ₛ(C₁-C₈)-alkyl, (C═O)ᵣS(O)ₙ(C₁-C₈)-alkyl, (C═O)ᵣS(O)ₙaryl, and heterocyclyl, with r and s at each occurrence being independently from each other 0 or 1, and n at each occurrence being 0, 1, 2 or 3;
W is a suitable functional group depending on the available site on the particular K_{V}1.3 inhibitor of interest which is attached to the linker;
Linker is selected from:
—(C(R⁹)(R¹⁰))_{I}—, wherein I is from 1 to 20; and R⁹ and R¹⁰ are independently of one another —H, halogen, —CF₃, —OH, —(C₁-C₆)-alkyl, —OC(O)(C₁-C₆)-alkyl, [(C═O)Oᵣ]ₛaryl, [(C═O)Oᵣ]ₛheteroaryl, or [(C═O)Oᵣ]ₛ(C₁-C₆)-alkyl, with r and s at each occurrence being independently from each other 0 or 1;
non-peptidic polymeric linkers selected from polyalkylene oxides, polyvinyl alcohol, polyvinylpyrrolidone as well as derivatives and copolymers thereof;
non-polymeric aliphatic linkers comprising a divalent, linear or branched, straight or cyclic, saturated or unsaturated hydrocarbon chain having from 2 to 20 carbon atoms, wherein the carbon atoms are optionally replaced by a group selected from —O—, —S—, —NH—, —C(═O)—, —OC(═O)—, —N(C₁-C₆ alkyl)-, NHC(═O)—, —N(C₁-C₆ alkyl)C(═O)—, —S(═O)— or —S(═O)₂— and wherein the chain is optionally substituted on carbon with one or more (e.g. 1, 2, 3 or 4) substituents;
a divalent radical formed from an amino acid or peptide; and 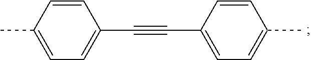
or a pharmaceutically acceptable salt, racemate, diastereomer, enantiomer, ester, carbamate, sulphate, phosphate or prodrug thereof.

2. The compound according to claim 1, wherein MTM is a mitochondria targeting moiety selected from: 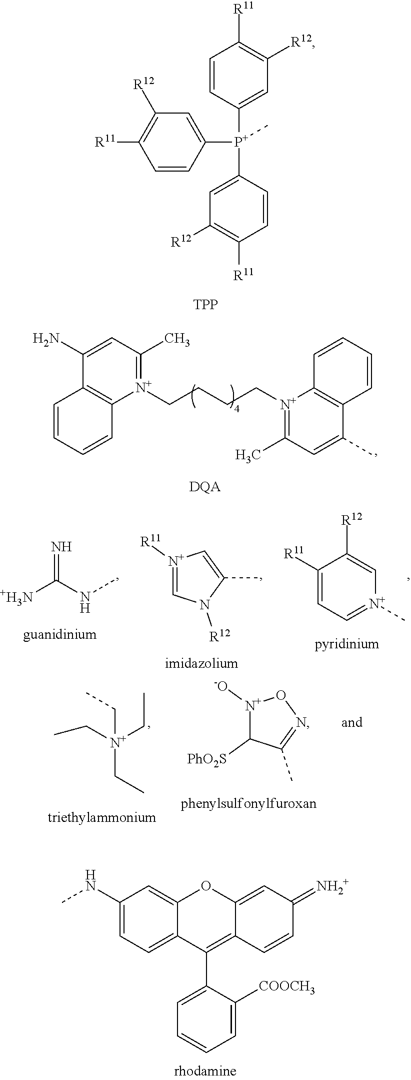
wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

3. The compound according to claim 1, wherein MTM is 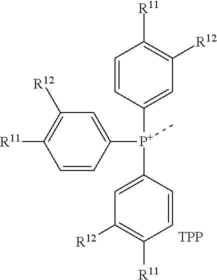
wherein R¹¹ and R¹² are independently of one another —H, halogen, —CF₃, —OH, —(C₁-C₆)-alkyl, —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

4. The compound according to claim 1, having structural Formula II or Formula III 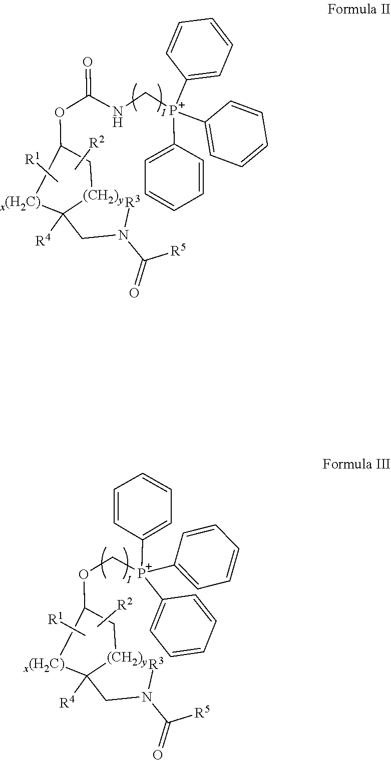
wherein I is from 1 to 20.

5. The compound according to claim 1, having structural Formula IV or Formula V 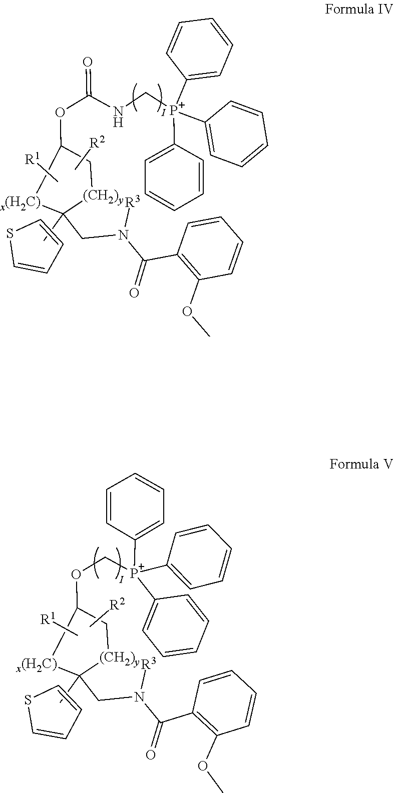
wherein I is from 1 to 20.

6. The compound according to claim 1, having structural Formula VI or Formula VII 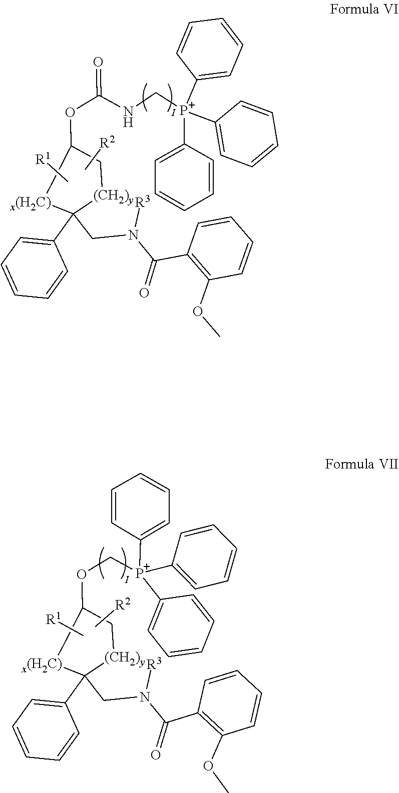
wherein I is from 1 to 20.

7. The compound according to claim 1, having structural Formula VIII or Formula IX 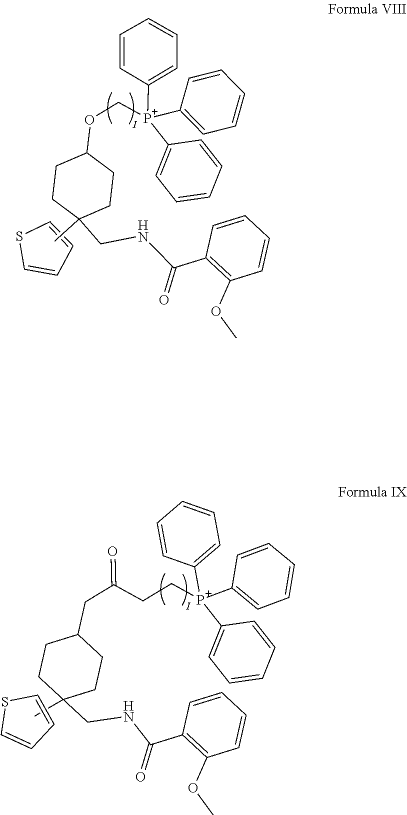
wherein I is from 1 to 20.

8. The compound according to claim 1, being an enantiomerically pure compound or an enantiomerically enriched compound with the following structural Formula X or Formula XI

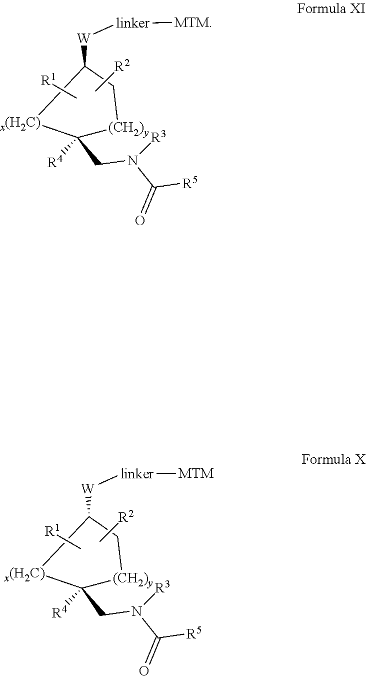

9. The compound according to claim 1, wherein W comprises a cleavable group selected from esters, carbamates, disulfide linkers, oxime linkers, hydrazine groups, diazolinkers, carbonyloxyethylsulfone groups, amino acid groups, and phenylacetamide groups.

10. The compound according to claim 1, wherein W is selected from (C₁-C₆)-alkyl, (C₂-C₆)-alkenyl, (C₂-C₆)-alkynyl, (C₃-C₆)cycloalkyl, aryl, heteroaryl, —OC(O)NR⁸, —COO—, —OC(O)—, —CONR⁸—, —NHR⁸—, —SO—, —SO₂NR⁸—, —CHR⁸—, —SO₂—, —CO—, —S—, —O—, —CH₂—, —OC(O)—CH₂—C(O)O—, and —CH(OH)—CH(OH)—;
wherein R⁸ is —H, —F, —Cl, —Br, —OH, —(C₁-C₆)-alkyl, or —OC(O)(C₁-C₆)-alkyl, [(C═O)Oᵣ]ₛaryl, or [(C═O)Oᵣ]ₛ(C₁-C₆)-alkyl, with r and s at each occurrence being independently from each other 0 or 1.

11. The compound according to claim 1, wherein R⁴ is an unsubstituted, monosubstituted, or disubstituted thiophene.

12. The compound of claim 1, wherein R⁵ is a substituted or unsubstituted phenyl.

13. The compound of claim 1, wherein R⁵ is 2-methoxyphenyl.

14. The compound of claim 1, wherein x is 2 and y is 1.

15. The compound of claim 1, wherein R¹ and R² are each hydrogen.

16. The compound of claim 1, wherein R³ is hydrogen.

17. The compound according to claim 1, which is selected from the group consisting of:
(3-(((((1R,4R)-4-((2-Methoxybenzamido)methyl)-4-(thiophen-3-yl)cyclohexyl)oxy)carbonyl)amino)propyl)triphenylphosphonium iodide;
(3-(((((1 S,4S)-4-((2-Methoxybenzamido)methyl)-4-(thiophen-3-yl)cyclohexyl)oxy)carbonyl)amino)propyl)triphenylphosphonium iodide;
(3-(((((1R,4R)-4-((2-Methoxybenzamido)methyl)-4-(thiophen-2-yl)cyclohexyl)oxy)carbonyl)amino)propyl)triphenylphosphonium iodide;
(3-(((((1 S,4S)-4-((2-Methoxybenzamido)methyl)-4-(thiophen-2-yl)cyclohexyl)oxy)carbonyl)amino)propyl)triphenylphosphonium iodide;
(3-(((((1R,4R)-4-((2-Methoxybenzamido)methyl)-4-phenylcyclohexyl)oxy)carbonyl)amino)propyl)triphenylphosphonium iodide;
(3-(((((1 S,4S)-4-((2-methoxybenzamido)methyl)-4-phenylcyclohexyl)oxy)carbonyl)amino)propyl)triphenylphosphonium iodide;
(4-(((((1R,4R)-4-((2-Methoxybenzamido)methyl)-4-(thiophen-3-yl)cyclohexyl)oxy)carbonyl)amino)butyl)triphenylphosphonium iodide;
(4-(((((1 S,4S)-4-((2-Methoxybenzamido)methyl)-4-(thiophen-3-yl)cyclohexyl)oxy)carbonyl)amino)butyl)triphenylphosphonium iodide;
(4-(((((1R,4R)-4-((2-Methoxybenzamido)methyl)-4-(thiophen-2-yl)cyclohexyl)oxy)carbonyl)amino)butyl)triphenylphosphonium iodide;
(4-(((((1 S,4S)-4-((2-Methoxybenzamido)methyl)-4-(thiophen-2-yl)cyclohexyl)oxy)carbonyl)amino)butyl)triphenylphosphonium iodide;
(4-(((((1R,4R)-4-((2-Methoxybenzamido)methyl)-4-phenylcyclohexyl)oxy)carbonyl)amino)butyl)triphenylphosphonium iodide; and
(4-(((((1 S,4S)-4-((2-Methoxybenzamido)methyl)-4-phenylcyclohexyl)oxy)carbonyl)amino)butyl)triphenylphosphonium iodide.

18. The compound of claim 1 for use in medicine.

19. A method of treating or preventing cancer in a warm-blooded animal comprising administering the compound of claim 1 to the warm-blooded animal.

20. The method according to claim 19, wherein the cancer is selected from the group consisting of breast cancer, colon cancer, prostate cancer, melanoma, smooth muscle cancer, skeletal muscle cancer, chronic lymphocytic leukemia, glioblastoma, and pancreatic ductal adenocarcinoma.

21. A pharmaceutical composition comprising a compound of claim 1, or a pharmaceutically acceptable salt or hydrate thereof, and a pharmaceutically acceptable excipient and/or carrier.

22. A process for preparing a compound as defined in claim 1 which process comprises:
Process step a) transformation of a compound of formula (XI) 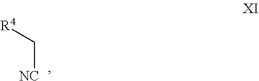
wherein R⁴ is as defined above,
to a compound of formula (XII) 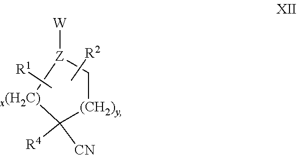
wherein R¹, R², R⁴, x and y are as defined above, Z is selected from CH or N, and W is selected from —H, hydroxyl, —NHR⁸, —COOH, —COO(C1-C6), —CHR⁸, —SO₃H or —SH,
and
Process step b) transformation of a compound of formula (XII) 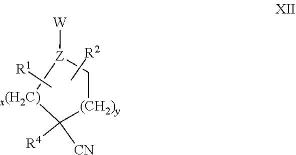
to a compound of formula (XIII) 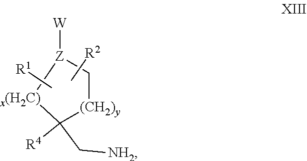
wherein R¹, R², R⁴, Z, W, x and y are as defined above,
and
Process step c) transformation of a compound of formula (XIII) 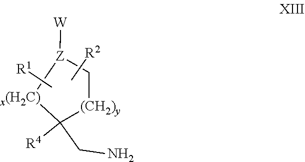
to a compound of formula (XIV) 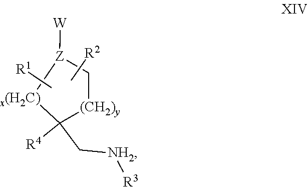
wherein R¹, R², R³, R⁴, Z, W, x and y are as defined above,
and
Process step d) reacting a compound of formula (XIV) with a compound of formula (XV): 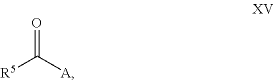
wherein A is selected from hydroxyl, alkoxy, halogen,
to a compound of formula (XVI): 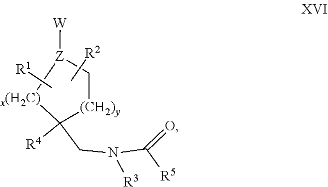
wherein R¹, R², R³, R⁴, R⁵, Z, W, x and y are as defined above,
and
Process step e) reacting a compound of formula (XVI) with a compound of formula (XVII): 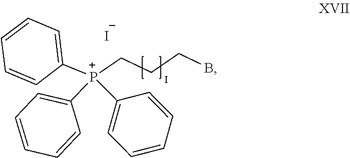
wherein B is selected from hydroxyl, mesyl, tosyl, halogen,
to a compound of formula (XVIII): 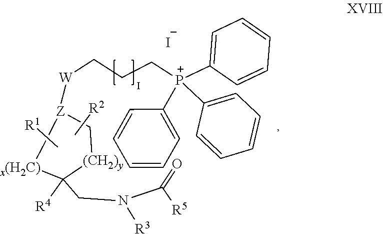
wherein R¹, R², R³, R⁴, R⁵, W, Z, x and y are as defined above.
